(19) United States
(12) Patent Application Publication
(10) Pub. No.: US 2026/0191807 A1
(43) Pub. Date: Jul. 9, 2026

(54) COMPOSITIONS AND METHODS FOR TREATMENT OF PRADER-WILLI SYNDROME

(71) Applicant: ERX Pharmaceuticals Inc., Cambridge, MA (US)

(72) Inventors: Teoman Uysal, Cambridge, MA (US); James E. Vath, Lynnfield, MA (US)

(21) Appl. No.: 19/130,589

(22) PCT Filed: Nov. 16, 2023
(86) PCT No.: PCT/US23/80136

§ 371 Date: May 16, 2025

Related U.S. Application Data
(60) Provisional application No. 63/384,193, filed on Nov. 17, 2022.

(51) Int. Cl. A61K 31/192; A61K 9/00; A61K 31/135; A61K 31/136; A61K 31/137; A61K 31/138; A61K 31/343; A61K 31/381; A61K 31/4168; A61K 31/4525; A61K 31/55; A61K 31/5513; A61K 31/5517; A61K 47/18; A61K 47/26; A61K 47/38; A61P 25/00

(52) U.S. Cl. CPC A61K 31/192; A61K 9/0043; A61K 31/135; A61K 31/136; A61K 31/137; A61K 31/138; A61K 31/343; A61K 31/381; A61K 31/4168; A61K 31/4525; A61K 31/55; A61K 31/5513; A61K 31/5517; A61K 47/186; A61K 47/26; A61K 47/38; A61P 25/00

(57) ABSTRACT

Described herein, inter alia, are compositions and methods for treating Prader-Willi Syndrome and psychiatric or mental disorders such as those associated with compulsive behaviors, e.g., hyperphagia or PWS-associated compulsive behaviors, using celastrol or a polymorph or analogue thereof. Further described herein are related kits and compositions.

### RELATED APPLICATIONS

This application claims priority to, and the benefit of, U.S. Provisional Application No. 63/384,193, filed on Nov. 17, 2022, the content of which is incorporated herein by reference in its entirety.

### BACKGROUND OF THE INVENTION

Obesity is often associated with some psychiatric disorders, such as Prader-Willi syndrome (PWS). PWS is a rare genetic disorder caused by loss of function on chromosome 15 reported in 1:15,000 births. In early childhood the disease manifests with a failure-to-thrive syndrome, and in late childhood, patients become uncontrollably hungry leading to obesity. Children affected by PWS also exhibit intellectual impairment and severe behavioral problems, mainly attributed to hyperphagia. Further, PWS was found to be associated with high rates of obsessive-compulsive symptoms. The patients are very difficult to manage and have a significantly shortened lifespan with an average age of death of 33 years. Similarly, there is a significant need to develop safe and effective therapies to treat such diseases and disorders.

### SUMMARY OF THE INVENTION

The invention provides a solution to the problem of treating psychiatric disorders such as those associated with compulsive behaviors, e.g., hyperphagia. Hyperphagia, defined as excessive appetite, is characterized by an extreme unsatisfied drive to consume food. It is associated with developmental impairment or behavioral management problems. For example, hyperphagia of PWS is characterized by stealing of food, hiding of food, binge-eating, nighttime eating and eating non-nutritive substances in addition to food items. Provided herein, inter alia, are compositions comprising compounds disclosed herein and methods of using the same for treatment of such disorders.

In one aspect, the disclosure features a method for treating a psychiatric or mental health disorder comprising
(i) identifying a subject having a compulsive behavior disorder and
(ii) administering a celastrol compound, a celastrol polymorph, or a celastrol analogue, or a pharmaceutical composition comprising a celastrol compound, a celastrol polymorph, or a celastrol analogue to the subject,
wherein a compulsive behavior associated with the compulsive behavior disorder is reduced following treatment.

In one aspect, the disclosure features a method for treating Prader-Willi Syndrome (PWS), comprising
(i) identifying a subject having PWS and
(ii) administering a composition comprising a celastrol compound, a celastrol polymorph, or a celastrol analogue to said subject,
wherein a PWS-associated compulsive behavior is reduced following treatment.

In one aspect, the disclosure features a method for treating a Prader-Willi Syndrome (PWS)-associated compulsive behavior comprising
(i) identifying a subject having PWS and
(ii) administering a composition comprising a celastrol compound, a celastrol polymorph, or a celastrol analogue to said subject,
wherein the PWS-associated compulsive behavior is reduced following treatment.

In one aspect, the disclosure features a method for treating a mental disease or disorder (e.g., a psychiatric or mental health disorder, e.g., a compulsive behavior disorder), wherein the mental disease or disorder includes compulsive behavior, comprising
(i) identifying a subject having a disorder that includes compulsive behavior and
(ii) administering a celastrol compound, a celastrol polymorph, or a celastrol analogue, or a pharmaceutical composition comprising a celastrol compound, a celastrol polymorph, or a celastrol analogue to the subject,
wherein the compulsive behavior is reduced following treatment.

In one aspect, the disclosure features a method for treating a disorder characterized by or associated with compulsive behavior comprising
(i) identifying a subject comprising a disorder characterized by or associated with compulsive behavior and
(ii) administering a celastrol compound, a celastrol polymorph, or a celastrol analogue, or a pharmaceutical composition comprising a celastrol compound, a celastrol polymorph, or a celastrol analogue to the subject,
wherein the compulsive behavior is reduced following treatment.

In one aspect, the disclosure features a method for treating a disease or disorder comprising hyperphagia comprising
(i) identifying a subject exhibiting hyperphagic behavior and
(ii) administering a celastrol compound, a celastrol polymorph, or a celastrol analogue, or a pharmaceutical composition comprising a celastrol compound, a celastrol polymorph, or a celastrol analogue to the subject,
wherein the hyperphagic behavior is reduced following treatment.

In one aspect, the disclosure features a method for treating hyperphagic behavior comprising
(i) identifying a subject exhibiting hyperphagic behavior and
(ii) administering a celastrol compound, a celastrol polymorph, or a celastrol analogue, or a pharmaceutical composition comprising a celastrol compound, a celastrol polymorph, or a celastrol analogue to the subject,
wherein the hyperphagic behavior is reduced following treatment.

In one aspect, the disclosure features a method for treating a mental disease or disorder (e.g., a psychiatric or mental health disorder, e.g., a compulsive behavior disorder) in a subject, comprising administering a celastrol compound, a celastrol polymorph, or a celastrol analogue, or a pharmaceutical composition comprising a celastrol compound, a celastrol polymorph, or a celastrol analogue to the subject.

In one aspect, the disclosure features a method for treating a psychiatric or mental health disorder in a subject, comprising administering a celastrol compound, a celastrol polymorph, or a celastrol analogue, or a pharmaceutical composition comprising a celastrol compound, a celastrol polymorph, or a celastrol analogue to the subject.

In one aspect, the disclosure features a method for treating a compulsive behavior disorder in a subject, comprising administering a celastrol compound, a celastrol polymorph, or a celastrol analogue, or a pharmaceutical composition comprising a celastrol compound, a celastrol polymorph, or a celastrol analogue to the subject.

In one aspect, the disclosure features a method for treating a disorder characterized by or associated with a compulsive behavior in a subject, comprising administering a celastrol compound, a celastrol polymorph, or a celastrol analogue, or a pharmaceutical composition comprising a celastrol compound, a celastrol polymorph, or a celastrol analogue to the subject.

In one aspect, the disclosure features a method of treating a Prader Willi Syndrome (PWS)-associated compulsive behavior in a subject comprising administering to a subject in need thereof a celastrol compound, a celastrol polymorph, or a celastrol analogue of the disclosure, or a pharmaceutical composition comprising a celastrol compound, a celastrol polymorph, or a celastrol analogue to the subject.

In one aspect, the disclosure features a method of treating Prader Willi Syndrome (PWS) in a subject comprising administering a celastrol compound, a celastrol polymorph, or a celastrol analogue of the disclosure, or a pharmaceutical composition comprising a celastrol compound, a celastrol polymorph, or a celastrol analogue to the subject.

In one aspect, the disclosure features a method of treating Prader Willi Syndrome (PWS) in a subject comprising administering celastrol, a polymorph of celastrol, or a celastrol analogue of the disclosure, or a pharmaceutical composition comprising celastrol, a polymorph of celastrol, or a celastrol analogue to the subject, wherein the treating PWS comprises reducing a compulsive behavior associated with PWS (e.g., hyperphagia associated with PWS).

In one aspect, the disclosure features a method of treating hyperphagia in a subject, comprising administering a celastrol compound, a celastrol polymorph, or a celastrol analogue of the disclosure, or a pharmaceutical composition comprising a celastrol compound, a celastrol polymorph, or a celastrol analogue to the subject.

In one aspect, the disclosure features a method of treating hyperphagic behavior in a subject, comprising administering a celastrol compound, a celastrol polymorph, or a celastrol analogue of the disclosure, or a pharmaceutical composition comprising a celastrol compound, a celastrol polymorph, or a celastrol analogue to the subject.

In one aspect, the disclosure features a method of promoting smoking cessation in a subject, comprising administering a celastrol compound, a celastrol polymorph, or a celastrol analogue of the disclosure, or a pharmaceutical composition comprising a celastrol compound, a celastrol polymorph, or a celastrol analogue to the subject.

In one aspect, the disclosure features a method of treating nicotine addiction in a subject, comprising administering a celastrol compound, a celastrol polymorph, or a celastrol analogue of the disclosure, or a pharmaceutical composition comprising a celastrol compound, a celastrol polymorph, or a celastrol analogue to the subject.

In one aspect, the disclosure provides a pharmaceutical composition for preventing or treating a mental disease or disorder (e.g., a psychiatric or mental health disorder, e.g., a compulsive behavior disorder) in a subject, comprising a celastrol compound, a celastrol polymorph, or a celastrol analogue to the subject. In some embodiments, the pharmaceutical composition may promote weight loss, reduce body fat, reduce food intake, improve homeostasis, or combinations thereof.

In one aspect, the disclosure provides a pharmaceutical composition for preventing or treating Prader-Willi Syndrome (PWS) or a Prader-Willi Syndrome (PWS)-associated compulsive behavior in a subject, comprising a celastrol compound, a celastrol polymorph, or a celastrol analogue. In some embodiments, the pharmaceutical composition may promote weight loss, reduce body fat, reduce food intake, improve homeostasis, or combinations thereof.

In one aspect, the disclosure provides a pharmaceutical composition for promoting smoking cessation in a subject, comprising a celastrol compound, a celastrol polymorph, or a celastrol analogue.

In one aspect, the disclosure provides a pharmaceutical composition for preventing or treating nicotine addiction in a subject, comprising a celastrol compound, a celastrol polymorph, or a celastrol analogue.

In one aspect, the disclosure provides a pharmaceutical composition for preventing or treating obesity in a subject, comprising a celastrol compound, a celastrol polymorph, or a celastrol analogue.

In some embodiments, a pharmaceutical composition of the disclosure (including pharmaceutical composition administered in a method of the disclosure) includes the active ingredient (e.g., celastrol, celastrol polymorph, or celastrol analogue) and an inactive component, e.g., a capsule, to facilitate the intended mode of administration, e.g., oral administration. In some embodiments, a pharmaceutical composition of the disclosure further comprises a pharmaceutically acceptable excipient. In some embodiments, a pharmaceutical composition of the disclosure does not comprise a pharmaceutically acceptable excipient. In some embodiments, when the pharmaceutical composition of the disclosure does not comprise a pharmaceutically acceptable excipient the pharmaceutical composition comprises the celastrol compound, celastrol polymorph, or celastrol analogue in the form of a powder packaged in a capsule.

In some embodiments, a pharmaceutical composition of the disclosure (including pharmaceutical composition administered in a method of the disclosure) is a liquid suspension. In some embodiments, a pharmaceutical composition of the disclosure (including pharmaceutical composition administered in a method of the disclosure, e.g., a liquid suspension) is a pharmaceutical composition for intranasal administration.

In some embodiments, the liquid suspension comprises a pharmaceutical agent, wherein the pharmaceutical agent is a celastrol compound, a celastrol polymorph, or a celastrol analogue and at least one of an emulsifier or surfactant (e.g., polysorbate 20), a preservative (e.g., benzalkonium chloride), and a mucoadhesive or viscosifier (e.g., hydroxypropyl methylcellulose (HPMC) or a combination of microcrystalline cellulose and sodium carboxymethyl cellulose (MCC-NaCMC)).

In some embodiments, a pharmaceutical composition of the disclosure (including pharmaceutical composition administered in a method of the disclosure) comprises:
(i) a pharmaceutical agent, wherein the pharmaceutical agent is a celastrol compound, a celastrol polymorph, or a celastrol analogue;
(ii) an emulsifier or surfactant; and
(iii) a mucoadhesive or viscosifier;
   wherein the pharmaceutical composition is in the form of a liquid suspension.

In some embodiments, a pharmaceutical composition of the disclosure (including pharmaceutical composition administered in a method of the disclosure) comprises:
(i) a pharmaceutical agent, wherein the pharmaceutical agent is a celastrol compound, a celastrol polymorph, or a celastrol analogue;
(ii) a polysorbate; and
(iii) a cellulose;
wherein the pharmaceutical composition is in the form of a liquid suspension.

In some embodiments, a pharmaceutical composition of the disclosure (including pharmaceutical composition administered in a method of the disclosure) comprises:
(i) a pharmaceutical agent, wherein the pharmaceutical agent is a celastrol compound, a celastrol polymorph, or a celastrol analogue;
(ii) an emulsifier or surfactant;
(iii) a mucoadhesive or viscosifier; and
(iv) a preservative.

In some embodiments, a pharmaceutical composition of the disclosure (including pharmaceutical composition administered in a method of the disclosure) comprises:
(i) a pharmaceutical agent, wherein the pharmaceutical agent is a celastrol compound, a celastrol polymorph, or a celastrol analogue;
(ii) a polysorbate;
(iii) a cellulose; and
(iv) a preservative.

In some embodiments, the liquid suspension comprises an emulsifier or surfactant (e.g., polysorbate 20) in an amount of from about 5 mg/mL to about 50 mg/mL (e.g., from about 5 mg/mL to about 40 mg/mL, from about 5 mg/mL to about 30 mg/mL, from about 10 mg/mL to about 30 mg/mL, or from about 20 mg/mL to about 30 mg/mL). In some embodiments, the liquid suspension comprises an emulsifier or surfactant (e.g., polysorbate 20) in an amount of about 25 mg/mL.

In some embodiments, the liquid suspension comprises a preservative (e.g., benzalkonium chloride) in an amount of up to about 1 mg/mL (e.g., up to about 0.1 mg/mL, up to about 0.2 mg/mL, up to about 0.3 mg/mL, up to about 0.4 mg/mL, up to about 0.5 mg/mL, up to about 0.6 mg/mL, up to about 0.7 mg/mL, up to about 0.8 mg/mL, or up to about 0.9 mg/mL). For example, in some embodiments, the liquid suspension comprises a preservative (e.g., benzalkonium chloride) in an amount of from about 0.01 mg/mL to 1.00 mg/mL (e.g., from about 0.01 mg/mL to about 0.8 mg/mL, from about 0.01 mg/mL to about 0.6 mg/mL, from about 0.01 mg/mL to about 0.4 mg/mL, from about 0.01 mg/mL to about 0.2 mg/mL, from about 0.05 mg/mL to about 0.2 mg/mL, or from about 0.05 mg/mL to about 0.15 mg/mL). In some embodiments, the liquid suspension comprises a preservative (e.g., benzalkonium chloride) in an amount of about 0.1 mg/mL.

In some embodiments, the liquid suspension comprises a mucoadhesive or viscosifier (e.g., HPMC or MCC-NaCMC) in an amount of from about 0.1 mg/mL to about 5 mg/mL (e.g, from about 0.1 mg/mL to about 4 mg/mL, from about 0.1 mg/mL to about 3 mg/mL, from about 0.1 mg/mL to about 2 mg/mL, or from about 0.5 mg/mL to about 1.5 mg/mL. In some embodiments, the liquid suspension comprises a mucoadhesive or viscosifier (e.g., HPMC or MCC-NaCMC) in an amount of about 1 mg/mL. In some embodiments, the liquid suspension comprises a mucoadhesive or viscosifier (e.g., HPMC or MCC-NaCMC) in an amount of from about 5 mg/mL to about 50 mg/mL (e.g, from about 5 mg/mL to about 40 mg/mL, from about 5 mg/mL to about 30 mg/mL, from about 10 mg/mL to about 30 mg/mL, from about 5 mg/mL to about 20 mg/mL, from about 10 mg/mL to about 20 mg/mL, or from about 15 mg/mL to about 20 mg/mL. In some embodiments, the liquid suspension comprises a mucoadhesive or viscosifier (e.g., HPMC or MCC-NaCMC) in an amount of about 18 mg/mL.

In some embodiments, the liquid suspension comprises a celastrol compound, a celastrol polymorph, or a celastrol analogue in an amount of from about 5 mg/mL to about 50 mg/mL (e.g, from about 5 mg/mL to about 40 mg/mL, from about 5 mg/mL to about 30 mg/mL, from about 10 mg/mL to about 30 mg/mL, or from about 15 mg/mL to about 25 mg/mL. In some embodiments, the liquid suspension comprises a celastrol compound, a celastrol polymorph, or a celastrol analogue in an amount of about 20 mg/mL.

In some embodiments, the liquid suspension comprises a celastrol compound, a celastrol polymorph, or a celastrol analogue and an emulsifier or surfactant (e.g., polysorbate 20) in a wt/wt ratio of from about 1:0.6 to about 1:2. For example, in some embodiments, the liquid suspension comprises a celastrol compound, a celastrol polymorph, or a celastrol analogue and an emulsifier or surfactant (e.g., polysorbate 20) in a wt/wt ratio of about 1:0.6, about 1:0.7, about 1:0.8, about 1:0.9, about 1:1.0, about 1:1.1, about 1:1.2, about 1:1.3, about 1:1.4, about 1:1.5, about 1:1.6, about 1:1.7, about 1:1.8, about 1:1.9, or about 1:2.0. In some embodiments, the liquid suspension comprises a celastrol compound, a celastrol polymorph, or a celastrol analogue and an emulsifier or surfactant (e.g., polysorbate 20) in a wt/wt ratio of about 1:1.3.

In some embodiments, the liquid suspension comprises a celastrol compound, a celastrol polymorph, or a celastrol analogue and a preservative (e.g., benzalkonium chloride) in a wt/wt ratio of from about 1:0.001 to about 1:0.01 (e.g., about 1:0.001, about 1:0.002, about 1:0.003, about 1:0.004, about 1:0.005, about 1:0.006, about 1:0.007, about 1:0.008, about 1:0.009, or about 1:0.01).

In some embodiments, the liquid suspension comprises a celastrol compound, a celastrol polymorph, or a celastrol analogue and a preservative (e.g., benzalkonium chloride) in a wt/wt ratio of about 1:0.005.

In some embodiments, the liquid suspension comprises a celastrol compound, a celastrol polymorph, or a celastrol analogue, an emulsifier or surfactant (e.g., polysorbate 20) and a preservative (e.g., benzalkonium chloride) in a wt/wt ratio of about 1:1.3:0.005.

In some embodiments, the liquid suspension comprises a celastrol compound, a celastrol polymorph, or a celastrol analogue and a mucoadhesive or viscosifier (e.g., HPMC or MCC-NaCMC) in a wt/wt ratio of from about 1:0.01 to about 1:0.1 (e.g., about 1:0.01, about 1:0.02, about 1:0.03, about 1:0.04, about 1:0.05, about 1:0.06, about 1:0.07, about 1:0.08, about 1:0.09, about 1:0.1). In some embodiments, the liquid suspension comprises a celastrol compound, a celastrol polymorph, or a celastrol analogue and a mucoadhesive or viscosifier (e.g., HPMC or MCC-NaCMC) in a wt/wt ratio of about 1:0.05. In some embodiments, the liquid suspension comprises a celastrol compound, a celastrol polymorph, or a celastrol analogue and a mucoadhesive or viscosifier (e.g., HPMC or MCC-NaCMC) in a wt/wt ratio of from about 1:0.3 to about 1:1.5 (e.g., about 1:0.3, about 1:0.4, about 1:0.5, about 1:0.6, about 1:7, about 1:0.8, about 1:0.9, about 1:1, about 1:1.1, about 1:1.2, about 1:1.3, about 1:1.4, or about 1:1.5). In some embodiments, the liquid suspension comprises a celastrol compound, a celastrol polymorph, or a celastrol analogue and a mucoadhesive or viscosifier (e.g., HPMC or MCC-NaCMC) in a wt/wt ratio of about 1:0.9.

In some embodiments, the liquid suspension comprises a celastrol compound, a celastrol polymorph, or a celastrol analogue, an emulsifier or surfactant (e.g., polysorbate 20) and a mucoadhesive or viscosifier (e.g., HPMC or MCC-NaCMC) in a wt/wt ratio of about 1:1.3:0.05. In some embodiments, the liquid suspension comprises a celastrol compound, a celastrol polymorph, or a celastrol analogue, an emulsifier or surfactant (e.g., polysorbate 20) and a mucoadhesive or viscosifier (e.g., HPMC or MCC-NaCMC) in a wt/wt ratio of about 1:1.3:0.9.

In some embodiments, the liquid suspension comprises a celastrol compound, a celastrol polymorph, or a celastrol analogue, an emulsifier or surfactant (e.g., polysorbate 20), a preservative (e.g., benzalkonium chloride), and a mucoadhesive or viscosifier (e.g., HPMC or MCC-NaCMC) in a wt/wt ratio of about 1:1.3:0.005:0.05. In some embodiments, the liquid suspension comprises a celastrol compound, a celastrol polymorph, or a celastrol analogue, an emulsifier or surfactant (e.g., polysorbate 20), a preservative (e.g., benzalkonium chloride), and a mucoadhesive or viscosifier (e.g., HPMC or MCC-NaCMC) in a wt/wt ratio of about 1:1.3:0.005:0.9.

In some embodiments, the liquid suspension comprises polysorbate 20 in an amount of from about 5 mg/mL to about 50 mg/mL (e.g., from about 5 mg/mL to about 40 mg/mL, from about 5 mg/mL to about 30 mg/mL, from about 10 mg/mL to about 30 mg/mL, or from about 20 mg/mL to about 30 mg/mL). In some embodiments, the liquid suspension comprises polysorbate 20 in an amount of about 25 mg/mL.

In some embodiments, the liquid suspension comprises benzalkonium chloride in an amount of up to about 1 mg/mL (e.g., up to about 0.1 mg/mL, up to about 0.2 mg/mL, up to about 0.3 mg/mL, up to about 0.4 mg/mL, up to about 0.5 mg/mL, up to about 0.6 mg/mL, up to about 0.7 mg/mL, up to about 0.8 mg/mL, or up to about 0.9 mg/mL). For example, in some embodiments, the liquid suspension comprises benzalkonium chloride in an amount of from about 0.01 mg/mL to 1.00 mg/mL (e.g., from about 0.01 mg/mL to about 0.8 mg/mL, from about 0.01 mg/mL to about 0.6 mg/mL, from about 0.01 mg/mL to about 0.4 mg/mL, from about 0.01 mg/mL to about 0.2 mg/mL, from about 0.05 mg/mL to about 0.2 mg/mL, or from about 0.05 mg/mL to about 0.15 mg/mL). In some embodiments, the liquid suspension comprises benzalkonium chloride in an amount of about 0.1 mg/mL.

In some embodiments, the liquid suspension comprises HPMC or MCC-NaCMC in an amount of from about 0.1 mg/mL to about 5 mg/mL (e.g, from about 0.1 mg/mL to about 4 mg/mL, from about 0.1 mg/mL to about 3 mg/mL, from about 0.1 mg/mL to about 2 mg/mL, or from about 0.5 mg/mL to about 1.5 mg/mL. In some embodiments, the liquid suspension comprises HPMC or

MCC-NaCMC in an amount of about 1 mg/mL. In some embodiments, the liquid suspension comprises HPMC or MCC-NaCMC in an amount of from about 5 mg/mL to about 50 mg/mL (e.g, from about 5 mg/mL to about 40 mg/mL, from about 5 mg/mL to about 30 mg/mL, from about 10 mg/mL to about 30 mg/mL, from about 5 mg/mL to about 20 mg/mL, from about 10 mg/mL to about 20 mg/mL, or from about 15 mg/mL to about 20 mg/mL. In some embodiments, the liquid suspension comprises HPMC or MCC-NaCMC in an amount of about 18 mg/mL.

In some embodiments, the liquid suspension comprises a celastrol compound, a celastrol polymorph, or a celastrol analogue in an amount of from about 5 mg/mL to about 50 mg/mL (e.g, from about 5 mg/mL to about 40 mg/mL, from about 5 mg/mL to about 30 mg/mL, from about 10 mg/mL to about 30 mg/mL, or from about 15 mg/mL to about 25 mg/mL. In some embodiments, the liquid suspension comprises a celastrol compound, a celastrol polymorph, or a celastrol analogue in an amount of about 20 mg/mL.

In some embodiments, the liquid suspension comprises a celastrol compound, a celastrol polymorph, or a celastrol analogue and polysorbate 20 in a wt/wt ratio of from about 1:0.6 to about 1:2. For example, in some embodiments, the liquid suspension comprises a celastrol compound, a celastrol polymorph, or a celastrol analogue and polysorbate 20 in a wt/wt ratio of about 1:0.6, about 1:0.7, about 1:0.8, about 1:0.9, about 1:1.0, about 1:1.1, about 1:1.2, about 1:1.3, about 1:1.4, about 1:1.5, about 1:1.6, about 1:1.7, about 1:1.8, about 1:1.9, or about 1:2.0. In some embodiments, the liquid suspension comprises a celastrol compound, a celastrol polymorph, or a celastrol analogue and polysorbate 20 in a wt/wt ratio of about 1:1.3.

In some embodiments, the liquid suspension comprises a celastrol compound, a celastrol polymorph, or a celastrol analogue and benzalkonium chloride in a wt/wt ratio of from about 1:0.001 to about 1:0.01 (e.g., about 1:0.001, about 1:0.002, about 1:0.003, about 1:0.004, about 1:0.005, about 1:0.006, about 1:0.007, about 1:0.008, about 1:0.009, or about 1:0.01).

In some embodiments, the liquid suspension comprises a celastrol compound, a celastrol polymorph, or a celastrol analogue and benzalkonium chloride in a wt/wt ratio of about 1:0.005.

In some embodiments, the liquid suspension comprises a celastrol compound, a celastrol polymorph, or a celastrol analogue, polysorbate 20, and benzalkonium chloride in a wt/wt ratio of about 1:1.3:0.005.

In some embodiments, the liquid suspension comprises a celastrol compound, a celastrol polymorph, or a celastrol analogue and HPMC or MCC-NaCMC in a wt/wt ratio of from about 1:0.01 to about 1:0.1 (e.g., about 1:0.01, about 1:0.02, about 1:0.03, about 1:0.04, about 1:0.05, about 1:0.06, about 1:0.07, about 1:0.08, about 1:0.09, about 1:0.1). In some embodiments, the liquid suspension comprises a celastrol compound, a celastrol polymorph, or a celastrol analogue and HPMC or MCC-NaCMC in a wt/wt ratio of about 1:0.05. In some embodiments, the liquid suspension comprises a celastrol compound, a celastrol polymorph, or a celastrol analogue and HPMC or MCC-NaCMC in a wt/wt ratio of from about 1:0.3 to about 1:1.5 (e.g., about 1:0.3, about 1:0.4, about 1:0.5, about 1:0.6, about 1:7, about 1:0.8, about 1:0.9, about 1:1, about 1:1.1, about 1:1.2, about 1:1.3, about 1:1.4, or about 1:1.5). In some embodiments, the liquid suspension comprises a celastrol compound, a celastrol polymorph, or a celastrol analogue and HPMC or MCC-NaCMC in a wt/wt ratio of about 1:0.9.

In some embodiments, the liquid suspension comprises a celastrol compound, a celastrol polymorph, or a celastrol analogue, polysorbate 20, and HPMC or MCC-NaCMC in a wt/wt ratio of about 1:1.3:0.05. In some embodiments, the liquid suspension comprises a celastrol compound, a celastrol polymorph, or a celastrol analogue, polysorbate 20, and HPMC or MCC-NaCMC in a wt/wt ratio of about 1:1.3:0.9.

In some embodiments, the liquid suspension comprises a celastrol compound, a celastrol polymorph, or a celastrol analogue, polysorbate 20, benzalkonium chloride, and HPMC or MCC-NaCMC in a wt/wt ratio of about 1:1.3:0.005:0.05. In some embodiments, the liquid suspension comprises a celastrol compound, a celastrol polymorph, or a celastrol analogue, polysorbate 20, benzalkonium chloride, and HPMC or MCC-NaCMC in a wt/wt ratio of about 1:1.3:0.005:0.9.

In one aspect, provided herein is a kit for preventing or treating a mental disease or disorder (e.g., a psychiatric or mental health disorder, e.g., a compulsive behavior disorder) in a subject, comprising a pharmaceutical composition described herein.

In one aspect, provided herein is a kit for preventing or treating a Prader-Willi Syndrome (PWS) or a Prader-Willi Syndrome (PWS)-associated compulsive behavior in a subject, comprising a pharmaceutical composition described herein.

In one aspect, provided herein is a kit for promoting smoking cessation in a subject, comprising a pharmaceutical composition described herein.

In one aspect, provided herein is a kit for preventing or treating nicotine addiction in a subject, comprising a pharmaceutical composition described herein.

In one aspect, provided herein is a kit for preventing or treating a mental disease or disorder, e.g., a psychiatric or mental health disorder, a compulsive behavior disorder, or a behavior disorder, in a subject, comprising pharmaceutical composition comprising a celastrol compound, a celastrol polymorph, or a celastrol analogue, an oral applicator, and instructions for use. In some embodiments, the mental disease or disorder is a disease associated with compulsive behavior and/or obesity as described herein. In some embodiments, the kit may promote weight loss, reduce body fat, reduce food intake, improve homeostasis, or combinations thereof. In some embodiments, the kit may be used for an oral administration or intraperitoneal administration of the pharmaceutical composition comprised therein. In some embodiments, a kit of the disclosure further comprises an oral applicator, and instructions for use. In some embodiments, a kit of the disclosure is used for an oral administration or intraperitoneal administration of the compositions of treating obesity.

In some embodiments, the kit described herein comprises guidelines or instructions for administration for the treatment of Prader-Willi syndrome, Binge eating disorder (BED), Autism, autism spectrum disorders (ASD), obsessive-compulsory disorder (OCD), depression, or a combination thereof. In some embodiments, the mental disease or disorder (e.g., a psychiatric or mental health disorder, e.g., a compulsive behavior disorder) or a disease characterized by compulsive behavior comprises Prader-Willi syndrome, Binge eating disorder (BED), autism, autism spectrum disorder (ASD), obsessive-compulsory disorder (OCD), depression, or a combination thereof.

In some embodiments, a mental disease or disorder or a disease characterized by compulsive behavior (e.g., a psychiatric or mental health disorder, e.g., a compulsive behavior disorder) comprises hyperphagia.

In some embodiments, the psychiatric disorder is a compulsive behavior disorder.

In some embodiments, the subject is human.

In some embodiments, the subject is a child. In some embodiments, the subject is an adult.

In some embodiments, the subject is 2 years of age or greater than 2 years of age. For example, in some embodiments, the subject is between 2 years of age and 100 years of age, inclusive of the endpoints. In some embodiments, the subject is between 2 years of age and 90 years of age, inclusive of the endpoints. In some embodiments, the subject is between 2 years of age and 80 years of age, inclusive of the endpoints. In some embodiments, the subject is between 2 years of age and 70 years of age, inclusive of the endpoints. In some embodiments, the subject is between 2 years of age and 60 years of age, inclusive of the endpoints. In some embodiments, the subject is between 2 years of age and 50 years of age, inclusive of the endpoints. In some embodiments, the subject is between 2 years of age and 40 years of age, inclusive of the endpoints. In some embodiments, the subject is between 2 years of age and 25 years of age, inclusive of the endpoints. In some embodiments, the subject is between 12 years of age and 100 years of age, inclusive of the endpoints. In some embodiments, the subject is between 12 years of age and 90 years of age, inclusive of the endpoints. In some embodiments, the subject is between 12 years of age and 80 years of age, inclusive of the endpoints. In some embodiments, the subject is between 12 years of age and 70 years of age, inclusive of the endpoints. In some embodiments, the subject is between 12 years of age and 60 years of age, inclusive of the endpoints. In some embodiments, the subject is between 12 years of age and 50 years of age, inclusive of the endpoints. In some embodiments, the subject is between 12 years of age and 40 years of age, inclusive of the endpoints. In some embodiments, the subject is between 12 years of age and 25 years of age, inclusive of the endpoints.

In some embodiments, the subject is not obese. In other embodiments, the subject is pre-obese, obese, or morbidly obese.

In some embodiments, the celastrol compound, the celastrol polymorph, or the celastrol analogue, or a pharmaceutical composition comprising the celastrol compound, the celastrol polymorph, or the celastrol analogue, is administered orally, in the form of a capsule or tablet.

In some embodiments, the celastrol compound, the celastrol polymorph, or the celastrol analogue, or a pharmaceutical composition comprising the celastrol compound, the celastrol polymorph, or the celastrol analogue, is administered by subcutaneous or intravenous administration. In some embodiments, the celastrol compound, the celastrol polymorph, or the celastrol analogue, or a pharmaceutical composition comprising the celastrol compound, the celastrol polymorph, or the celastrol analogue is administered by intranasal administration or transdermal administration.

In some embodiments, the celastrol compound, the celastrol polymorph, or the celastrol analogue, or a pharmaceutical composition comprising the celastrol compound, the celastrol polymorph, or the celastrol analogue is administered to the subject using oral administration, intravenous administration, subcutaneous administration, intranasal administration, transdermal administration, topical administration, parenteral administration, intraperitoneal administration, intramuscular administration, intrathecal administration, intralesional administration, intracranial administration, intraocular administration, intracardiac administration, intravitreal administration, intraosseous administration, intracerebral administration, intraarterial administration, intraarticular administration, intradermal administration, transmucosal administration, sublingual administration, enteral administration, sublabial administration, insufflation administration, suppository administration, inhaled administration.

In some embodiments, when the celastrol compound, the celastrol polymorph, or the celastrol analogue, or a pharmaceutical composition comprising the celastrol compound, the celastrol polymorph, or the celastrol analogue is administered to the subject via intranasal administration, the celastrol compound, the celastrol polymorph, or the celastrol analogue, or the pharmaceutical composition comprising the celastrol compound, the celastrol polymorph, or the celastrol analogue is formulated as an intranasal dosage form. In some embodiments, such dosage forms comprise solutions, suspensions, emulsions, powders, and gel compositions for intranasal delivery. In some embodiments, an intranasal dosage form is administered via a nasal spray, a dropper bottle, or a metered dose spray pump. In some embodiments, the nasal spray is an aqueous nasal spray, hydroalcoholic nasal spray, or nonaqueous-based nasal spray. In some embodiments, an intranasal dosage form is administered via a dry powder nasal spray, dry-powder inhaler, or aspirator.

In some embodiments, when the celastrol compound, the celastrol polymorph, or the celastrol analogue, is administered to the subject using intranasal administration, the celastrol compound, the celastrol polymorph, or the celastrol analogue is administered to the subject a dose of from about 0.001 mg to about 10 mg per kg of body weight (e.g. from about 0.001 mg to about 0.5 mg per kg of body weight).

In some embodiments, when a pharmaceutical composition comprising the celastrol compound, the celastrol polymorph, or the celastrol analogue is administered to the subject via intranasal administration, the pharmaceutical composition comprising the celastrol compound, the celastrol polymorph, or the celastrol analogue is administered to the subject in an amount corresponding to a dose of from about 0.001 mg to about 10 mg of the celastrol compound, the celastrol polymorph, or the celastrol analogue per kg of body weight (e.g. from about 0.001 mg to about 0.5 mg of the celastrol compound, the celastrol polymorph, or the celastrol analogue per kg of body weight).

In some embodiments, the celastrol compound, the celastrol polymorph, or the celastrol analogue, or a pharmaceutical composition comprising the celastrol compound, the celastrol polymorph, or the celastrol analogue are administered daily, every two days, every three days, every four days, every five days, or every six days.

In some embodiments, the celastrol compound, the celastrol polymorph, or the celastrol analogue, or a pharmaceutical composition comprising the celastrol compound, the celastrol polymorph, or the celastrol analogue are administered weekly, every two weeks, every three weeks, or monthly.

In some embodiments, the celastrol compound, the celastrol polymorph, or the celastrol analogue, or a pharmaceutical composition comprising the celastrol compound, the celastrol polymorph, or the celastrol analogue are administered once per day, twice per day, three times per day, or four times per day.

In some embodiments, the celastrol compound, the celastrol polymorph, or the celastrol analogue, or a pharmaceutical composition comprising the celastrol compound, the celastrol polymorph, or the celastrol analogue are administered once per week, twice per week, three times per week, four times per week, five times per week, or six times per week.

In some embodiments, the methods of the disclosure comprise identifying or diagnosing a subject that is in need of a treatment disclosed herein (e.g., diagnosing a subject with hyperphagia or PWS). For example, in some embodiments, the methods of the disclosure comprise identifying or diagnosing a subject with a psychiatric or mental health disorder such as compulsive behavior. In some embodiments, the methods of the disclosure comprise identifying a subject that is characterized by a compulsive behavior disorder, or a disorder characterized by compulsive behavior. For example, in some embodiments, the methods of the disclosure include administering a Hyperphagia Questionnaire. In some embodiments, the Hyperphagia Questionnaire is used to assess hyperphagia, e.g. in a subject for Prader-Willi Syndrome (PWS). For example, a compulsive behavior such as hyperphagia is diagnosed by a variety of methods including a Hyperphagia Questionnaire (HQ), e.g., Dyken's HQ or a modification or variation thereof as described below. Further, in some embodiments, the Dykens-based HQCT or a modified version thereof is used to monitor treated subjects. For example, an exemplary favorable clinical outcome is characterized by a score of 11+ down to 0, 1 or 2 following treatment.

Treatment of subjects as described herein leads to an improvement in the subject's disorder. For example, in some embodiments, the compulsive behavior is reduced by at least 10% following treatment, e.g., a subject Dyken's Hyperphagia Questionnaire (HQ) score of 11 or greater is reduced to 0, 1, or 2.

In some embodiments, the celastrol analogue is a compound comprising at least one structural modification to celastrol.

In some embodiments, the celastrol analogue is a compound of Formula (I):

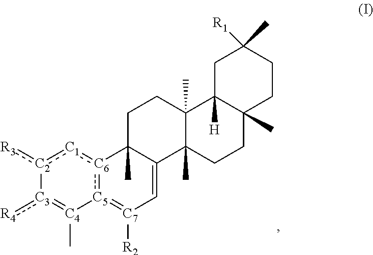

or a pharmaceutically acceptable salt, prodrug, or polymorph thereof, wherein
the dotted lines between C₁ and C₂, C₂ and R₃, C₃ and R₄, C₅ and C₆, C₅ and C₇, C₁ and C₆, and C₃ and C₄ indicate that a single or double bond may be present, as valence permits;
R₁ is —CN, —COOH, —COOCH₂CH₃, —CONHR₅, —CONR₅R₅, —COOR₅, —COOCH₃, —CH₂NR₅R₅, —CH₂OCONR₅R₅, —CH₂NR COOR₅, —CH₂R₅, —CH₂NR₅CONR₅R₅, —CH₂OH, —CH₂OR₅, alkylsulfate, alkylsulfonate, alkylphosphate, —CH₂OSO₃R₅, —CH₂OSO₂R₅, —CH₂OPO₃R₅R₅, —CH₂OPO₃HR₅, —CH₂OPO₃H₂, —C(═NR₅)NR₅R₅, —NR₅C(═NR₅)NR₅R₅, —CONH₂, —CH₂CONR₅R₅, —SR₅, —SO₃R₅, —SO₂R₅, —CH₂NHCOR₅, —CH₂NHCNR₅NR₅R₅, —CH₂COSR₅, CH₂NR₅COR₅, —CH₂NR₅CNR₅NR₅R₅, —CH₂NR₅COSR₅, —CH₂NHSO₂R₅, —CH₂NR₅SO₂R₅, —CHNR₅, —CHNOR₅, —H, —NH₂, —NHR₅, —NR₅R₅, —OH, —OR₅, phosphate, —OPO₃R₅R₅, —OPO₃HR₅, —OPO₃H₂, —NCO, —NCS, —N₃, —R₅, —C≡CR₅, —(CH═CH)R₅, —SH, —SR₅, —SO₂H, —SO₃H, —SO₂NR₅R₅, —SO₃R₅, —NHCOR₅, —, NHCNR₅NR₅R₅, —NHCOSR₅, secondary amide, tertiary amide, —NR₅COR₅, —NR₅C(═NH)NR₅R₅, —NR₅COSR₅, —NHC(═NR₅)R₅, —NR₅C(═NR₅)R₅, —NHSO₂ (NH₂), —NHSO₂R₅, —NR₅SO₂R₅, —NR₅SO₂NR₅R₅, —OCOR₅, —OCONR₅R₅, —O(C═O)OR₅, —SCOR₅, —O(C═NH)NR₅R₅, —OCSNHR₅, —OS(═O₂)R₅, —OS(═O₂)NR₅R₅, —SCONR₅R₅, —CH₂-aryl, —CH₂-heteroaryl,

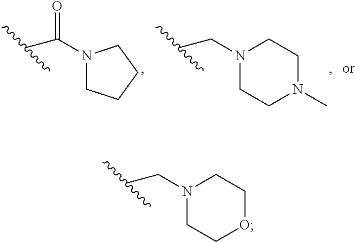

R₂ is —H, —CH₃, —SCH(CH₃)₂, —SC(═O)CH₃, —SC(═O)R₅, —SCH₂CH₂OCOCH₃, —SR₅, —SOR₅, —SOOR₅, —SCONR₅R₅,

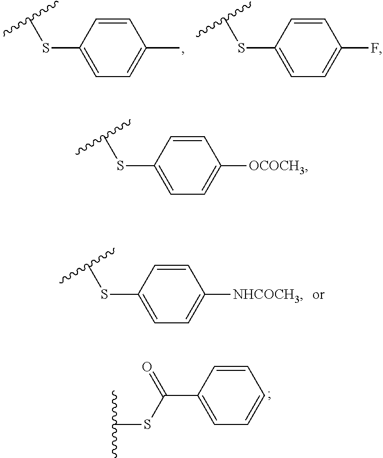

R₃ is —OCOCH₃, —OCOOCH₂CH₃, —OR₇, —R₇, or —NR₅R₅ when a double bond is present between C₁ and C₂, C₃ and C₄, and C₅ and C₆
R₄ is —OCOCH₃, —OCOOCH₂CH₃, —OR₇, —R₇, or —NR₅R₅ when a double bond is present between C₁ and C₂, C₃ and C₄, and C₅ and C₆;
R₃ is O when R₄ is O and a double bond is present between C₂ and R₃ and C₃ and R₄;
R₄ is —OCH₃, —OP(═O)(OCH₃)₂, —OH, —OCOOCH₂CH₃, —OCONHCH₂CH₃, —OCOOCH(CH₃)₂, —OR₇, —R₇, or —NR₅R₅ when R₃ is O and a double bond is present between C₂ and R₃; R₃ and R₄ may also be combined to form a heterocylic or carbocyclic ring;
R₅ is independently selected for each occurrence selected from hydrogen, an alkyl, cycloalkyl, alkoxy, heterocycloalkyl, alkylaryl, alkenyl, alkynyl, aryl, amine, and heteroaryl, wherein the heteroaryl is optionally substituted with substituents individually selected from alkyl, alkoxy, cycloalkyl, ether, halogen, hydroxyl, ether, cyano, nitrile, CF₃, ester, amide, cycloalkyl amide, sugar, urea, carbamate, thioether, sulfate, sulfonyl, sulfonic acid carboxylic acid, aryl, amine optionally substituted with one or more alkyl, and heteroarylamide optionally substituted with alkyl and/or alkoxy; or two R₅ groups taken together to form a cycloalkyl, heterocycloalkyl, aryl or heteraryl group, optionally substituted with substituents individually selected from alkyl, cycloalkyl, alkoxy, heterocycloalkyl, alkylaryl, alkenyl, alkynyl, aryl, heteroaryl, amine, halogen, hydroxyl, ether, nitrile, cyano, nitro, CF₃, ester amide, urea, carbamate, thioether, and a carboxylic acid group; and
R₇ is hydrogen, an alkyl, cycloalkyl, heterocycloalkyl, alkylaryl, alkenyl, alkynyl, aryl, or heteroaryl, optionally substituted with substituents individually selected from alkyl, cycloalkyl, ether, amine, halogen, hydroxyl, ether, nitrile, cyano, nitrile, CF₃, ester, amide, urea, carbamate, thioether, and carboxylic acid.

In some embodiments, R₁ is —NR₅C(═NR₅)NR₅R₅, —SR₅, —SO₃R₅, —SO₂R₅, —NH₂, —NHR₅, —NR₅R₅, —OH, —OR₅, —NCO, —NCS, —N₃, —SH, —SR₅, —SO₂H, —SO₃H, —SO₂NR₅R₅, —SO₃R₅, —NHCOR₅, —NHCNR₅NR₅R₅, —NHCOSR₅, —NR₅COR₅, —NR₅C(═NH)NR₅R₅, —NR₅COSR₅, —NHC(═NR₅)R₅, —NR₅C(═NR₅)R₅, —NHSO₂ (NH₂), —NHSO₂R₅, —NR₅SO₂R₅, —NR₅SO₂NR₅R₅, —OCOR₅, —OCONR₅R₅, —O(C═O)OR₅, —SCOR₅, —O(C═NH)NR₅R₅, —OCSNHR₅, —OS(═O₂)R₅, —OS(═O₂)NR₅R₅, or —SCONR₅R₅.

In some embodiments, the compounds of Formula (I) include those which are prodrugs.

In some embodiments, R₂ is H.

In some embodiments, R₂ is H.

In some embodiments, R₄ is —OH, —OR₇, or —R₇ when R₃ is O and a double bond is present between C₂ and R₃.

As used herein the celastrol compound is understood to be a compound having the structure of Formula II:

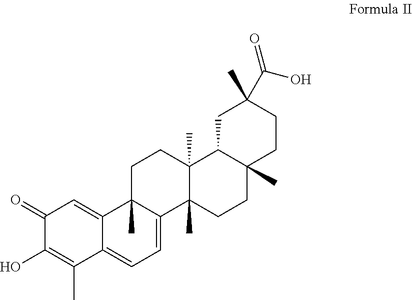

or a pharmaceutically acceptable salt, or prodrug thereof.

Further celastrol analogues are described in U.S. Pat. No. 10,662,218, which is incorporated herein by reference in its entirety.

Celastrol polymorphs are described in U.S. Provisional patent application Ser. No. 63/384,153 and in International Patent Application No. PCT/US23/80122, each of which is hereby incorporated by reference in its entirety.

The compounds described by Formula (I), (I)-a, (I)-b, (I)-c, or (II) as described in the enumerated embodiments can be prepared using methods known in the art and described herein. For example, celastrol can be obtained from commercial sources, or isolated from plants, e.g. Tripterygium, by methods known in the art (Kutney et al, Can. J. Chem. 59:2677, 1981) and Zhang et al, Acta Pharm. Sin. 212:592, 1986). Celastrol can be modified to render compounds of Formula (I), (I)-a, (I)-b, (I)-c, or Formula (II). Prepared compounds are purified using conventional methods to obtain compounds free of impurities. Prepared compounds are >75, >80, >85, >90, >95, >96, >97, >98, >99, >99.5% pure. Optionally, preferred compounds are >99% pure.

In some embodiments, a pharmaceutical composition of the disclosure is in the form of a capsule or tablet and is administered to the subject by oral administration. For example, a celastrol (or polymorph or analogue) powder is added into capsule, with or without an excipient and administered to subjects orally. Doses are described herein and are tailored by the physician or medical provider for the subject/patient being treated with lower doses for children.

In some embodiments, a pharmaceutical composition of the disclosure further comprises a pharmaceutically acceptable excipient. In other embodiments, a pharmaceutical composition of the disclosure does not comprise an excipient. For example, in some embodiments, a pharmaceutical composition of the disclosure is a celastrol compound, celastrol polymorph, or celastrol analogue in powder form packaged in, e.g., a capsule with no additional excipients. For example, the dosage unit comprises a celastrol compound, celastrol polymorph, or celastrol analogue in powder form in a capsule.

Examples of pharmaceutically acceptable excipients include, at least, Solutol®, dimethylacetamide (DMAc or DMA), water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention. In some embodiments, the pharmaceutically acceptable excipient described herein may include at least one of Solutol®, dimethylacetamide (DMAc or DMA), etc.

In some embodiments, a method of disclosure further comprises administering one or more additional therapeutic agent, wherein the one or more additional therapeutic agents comprises an anti-anxiety or anti-depression agent. In some embodiments, a pharmaceutical composition or the kit of the disclosure further comprises one or more additional therapeutic agents, wherein the one or more additional therapeutic agents comprises an anti-anxiety or anti-depression agent. For example, in some embodiments, the anti-anxiety or anti-depression agent comprises a serotonin selective reuptake inhibitor (SSRI), a serotonin norepinephrine reuptake inhibitor (SNRI), a benzodiazepine (BZD), a tricyclic antidepressant (TCA), a monoamine oxidase (MAO) inhibitor (MAOI), or an antihistamine. Non-limiting examples of an anti-anxiety agent include escitalopram, fluoxetine, fluvoxamine, paroxetine, sertraline, duloxetine, venlafaxine, alprazolam, chlordiazepoxide, clonazepam, diazepam, lorazepam, oxazepam, clomipramine, doxepine, imipramine, phenelzine, hydroxyzine, or buspirone. In examples that include an anti-depression agent, the anti-depression agent comprises fluoxetine hydrochloride (PROZAC®), amitriptyline, or clonidine hydrochloride.

The table below summarizes exemplary common compounds useful for co-administration with a celastrol compound, celastrol polymorph, or celastrol analogue:

| Activity | Compounds/Therapeutic Agent |
|---|---|
| Anxiety/antidepressant medications | Fluoxetine hydrochloride |
| | (e.g., PROZAC ®) |
| | Amitriptyline |
| | Clonidine hydrochloride |
| Sleep medications/Sleep related/ | Melatonin (e.g., modified-release) |
| Stimulants | Methylphenidate hydrochloride |
| | Methylphenidate hydrochloride, |
| | (e.g., extended-release) |
| | Dexamfetamine sulphate |
| | Modafinil |
| Anti-psychotic medications for | Risperidone |
| behavior or psychiatric disorders/ | Aripiprazole |
| mood stabilizers | Ziprasidone |
| | Olanzapine |
| | Quetiapine |
| | Haloperidol |
| | Lithium Carbonate |
| Anti-convulsant/anti-epileptic | Valproic Acid (e.g., Sodium |
| medications for mood stabilizing | Valproate) |
| or behavior | Lamotrigine |
| | Topirimate |
| Benzodiazepines (e.g., fast acting | Lorazepam (e.g., Ativan) |
| anxiety reducing sedatives) | |
| Growth Hormone | Somatropin |
| | Genotropin |
| | Omnitrope |
| | Norditropin ® FlexPro |

Other drugs or agents that may be co-administered include exemplary conmeds described below.

In some embodiments, the celastrol compound, the celastrol polymorph, or the celastrol analogue and the one or more additional therapeutic agents are formulated in the same dosage unit. In some embodiments, the celastrol compound, the celastrol polymorph, or the celastrol analogue and the one or more additional therapeutic agents are formulated in separate dosage units. In some embodiments, the celastrol compound, the celastrol polymorph, or the celastrol analogue and the one or more additional therapeutic agents are administered simultaneously. In some embodiments, the celastrol compound, the celastrol polymorph, or the celastrol analogue and the one or more additional therapeutic agents are administered sequentially.

In some embodiments of the method of the disclosure, the celastrol compound, a celastrol polymorph, or a celastrol analogue is administered to the subject at a dose of from about 0.005 mg to about 10 mg per kg of body weight. For example, in some embodiments the celastrol compound, a celastrol polymorph, or a celastrol analogue is administered to the subject at a dose of from about 0.5 mg to 8 mg.

In some embodiments of the method of the disclosure, the celastrol compound, a celastrol polymorph, or a celastrol analogue is administered to the subject at a dose of from about 0.005 mg to about 0.5 mg per kg. In some embodiments of the method of the disclosure, the celastrol compound, a celastrol polymorph, or a celastrol analogue is administered to the subject at a dose of from about 0.5 mg to about 4 mg or from about 0.5 to about 8 mg. For example (assuming a 100 kg obese subject on average), a dose of 8 mg is equivalent to 0.08 mg/kg, i.e., within the dose range of 0.005 mg to about 0.5 mg per kg described above. Clinical regimens include those in which the subject takes a tablet, capsule, or other formulation comprising the above-described dose under a typical regimen of once a week or twice a week. Other dosing regimens, e.g., more frequently than once or twice a week, e.g., 3, 4, 6, or 7 times a week or less frequently than once or twice a week, e.g., once every 8, 9, 10, 11, 12, 13, 14, 15, 20, 21, 28 30 or 31 days (such as once a month) are within the discretion of a medical practitioner.

In some embodiments of the method of the disclosure, the celastrol compound, a celastrol polymorph, or a celastrol analogue is administered to the subject at a dose of from about 0.1 to about 0.5 mg/kg, from about 0.1 to about 1 mg/kg, from about 0.1 to about 5 mg/kg, from about 0.1 to about 10 mg/kg, from about 0.5 to about 10 mg/kg, from about 0.5 to about 5 mg/kg, or from about 1 to about 5 mg/kg. In some embodiments, the pharmaceutical formulation is administered to the subject in a dose of from about 0.5 to about 2 mg/kg. For example, an exemplary dosage (e.g., once or twice a week) is from about 0.5 mg to about 8 mg for a 100 kg human subject, e.g., from about 0.005 to about 0.04 mg/kg. For example, the dosage range may be about 0.001 to about 1 mg/kg, e.g., 0.005 to about 0.2 mg/kg. In some embodiments, the celastrol compound, a celastrol polymorph, or a celastrol analogue is administered to the subject once or twice per week. Without wishing to be bound by theory, in some cases less frequent administration (e.g. once or twice per week instead of e.g., daily) is beneficial, especially in the treatment of children.

An exemplary dose range is on the lower range, e.g., from about 0.005 mg to about 0.5 mg per kg. For example, the capsule is administered in from about 0.5 mg to about 4 mg doses (in a day) or in from about 0.5 mg to about 8 mg doses (in a day). Assuming an 100 kg subject on average, an 8 mg dose will be 0.08 mg/kg, i.e., within the preferred dose range. For example, an exemplary target range for a subject is 0.5 to 8 mg total dose once or twice a week.

In some embodiments, the methods or pharmaceutical compositions of the disclosure induce weight loss, reduce body fat, reduce food intake, improve glucose homeostasis, and/or prevent an increase in the body mass index of the subject. Measurement of the foregoing parameters are useful to monitor patient response to treatment. In some embodiments, the methods or pharmaceutical compositions of the disclosure reduce a subject's body weight, body fat, food intake, or BMI to about 95%, 90%, 85%, 80%, 75%, 70%, 65%, 60%, 55%, 50%, 45%, 40%, 35%, 30%, 25%, 20%, 15%, 10%, or lower (and all sub-values and sub-ranges there between, including endpoints), compared to a subject that has not been treated with a method according to the disclosure or has not been administered a pharmaceutical composition of the disclosure. In some embodiments, the methods or pharmaceutical compositions of the disclosure decrease the degree of the compulsive behavior in the subject by about 10%, 15%, 20%, 25%, 30%, 35%, 40%, 45%, 50%, 55%, 60%, 65%, 70%, 75%, 80%, 85%, 90%, 95%, or more (and all sub-values and sub-ranges there between, including endpoints). In some embodiments, the methods or pharmaceutical compositions of the disclosure decrease the degree of the compulsive behavior by at least 10%, at least 15%, or at least 20%.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting

### BRIEF DESCRIPTION OF THE DRAWINGS

FIG. 1 is a line graph showing daily body weights of C57BL/6 DIO mice during treatments with Beloranib, celastrol, or vehicle control.

FIG. 2 is a line graph showing daily body weights (in percentage of the body weight at day 1) of C57BL/6 DIO mice during treatments with Beloranib, celastrol, or vehicle control.

FIG. 3 is a line graph showing food intake data from the study for FIGS. 1 and 2.

FIG. 4 is a line graph showing the cumulative food intake data from the study for FIGS. 1-3 (****: p≤0.0001).

FIG. 5 is a bar graph comparing nestlet shredding behavior of C57BL/6 DIO mice treated as in FIGS. 1-4, with vehicle control (1), beloranib (2), and celastrol (3), measured over 12 hours on day −1, day −6, day −11, and day −15 of study. Statistics compare the treatment groups to vehicle on the corresponding day. (*: p≤0.05, ****: p≤0.0001).

FIG. 6 is a bar graph comparing nestlet shredding behavior of C57BL/6 DIO mice treated as in FIGS. 1-5, with vehicle control (1), beloranib (2), and celastrol (3), measured over 24 hours on day −1, day −6, day −11 and day −15 of study. Statistics compare the treatment groups to vehicle on the corresponding day. (****: p≤0.0001) FIG. 7 is a line graph showing daily body weights of C57BL/6 Magel2 mice during treatments with celastrol or vehicle control. In the graph (−/+) denotes mice heterozygous for Magel2 and (−/−) denotes mice that are homozygous for Magel2.

FIG. 8 is a line graph showing daily body weights (in percentage of the body weight at day 1) of Magel2 mice during treatments with celastrol or vehicle control. In the graph (−/+) denotes mice heterozygous for Magel2 and (−/−) denotes mice that are homozygous for Magel2.

FIG. 9 is a line graph showing food intake data from the study for FIGS. 7 and 8.

FIG. 10 is a bar graph comparing nestlet shredding behavior of C57BL/6 Magel2 mice treated as in FIGS. 7-9, with vehicle control (1), beloranib (2), and celastrol (3), measured over 12 hours on day 1, day 11, and day 21 of study. Statistics compare the treatment groups to vehicle on the corresponding day. (ns—not significant, ****p≤0.0001).

FIG. 11 is a bar graph comparing nestlet shredding behavior of C57BL/6 Magel2 mice treated as in FIGS. 7-9, with vehicle control (1), beloranib (2), and celastrol (3), measured over 24 hours on day 1, day 11, and day 21 of study. Statistics compare the treatment groups to vehicle on the corresponding day. (ns—not significant, ***p≤0.001).

FIG. 12 is a graph summarizing the concentration of celastrol in plasma of male Sprague Dawley rats following intranasal dosing of a liquid intranasal formulation (Intranasal A as described in Example 3) at a dose concentration of 2 mg/kg. The numbers identify the tested rats (rats number 5, 6, 7, and 8).

FIG. 13 is a graph summarizing the concentration of celastrol in plasma of male Sprague Dawley rats following intranasal dosing of a liquid intranasal formulation (Intranasal B as described in Example 3) at a dose concentration of 2 mg/kg. The numbers identify the tested rats (rats number 9, 10, 11, and 12).

FIG. 14 is a graph summarizing the concentration of celastrol in plasma of male Sprague Dawley rats following oral dosing of a celastrol drug substance (comprising a crystalline Form III of celastrol) at a dose concentration of 2 mg/kg. The numbers identify the tested rats (rats number 1, 2, 3, and 4).

### DETAILED DESCRIPTION OF THE INVENTION

Exemplary psychiatric diseases/disorders to be treated in accordance with the invention are described below.

The methods, pharmaceutical compositions, and kits disclosed herein, can reduce body weight, food intake, anxiety and compulsive behaviors. For example, as demonstrated in Example 1, an exemplary celastrol compound, celastrol polymorph, or celastrol analogue of the disclosure was found to have significant effects on body weight, food intake, and 12-hour nestlet shredding (an art-recognized model for compulsive behaviors) in C57BL/6 DOI mice (e.g., C57BL/6 DOI mice). Further, an exemplary celastrol compound, celastrol polymorph, or celastrol analogue of the disclosure was found to affect body weight, food intake, and 12-hour nestlet shredding in C57BL/6 Magel 2 mice, which are a model for PWS.

As also demonstrated in Example 1, an exemplary celastrol compound, celastrol polymorph, or celastrol analogue of the disclosure was found to reduce body weight, food intake, and 12-hour nestlet shredding in C57BL/6 DOI mice more significantly than beloranib. Further, as shown in Example 1 nestlet shredding was also reduced in C57BL/6 Magel 2 mice.

### Advantages of Celastrol Over Other PWS Treatments

Without wishing to be boud by theory, and as demonstrated in Example 1, the compounds of the disclosure are useful for the treatment of PWS and other conditions that present with anxiety and compulsive behaviors. For example, the methods, pharmaceutical compositions, and kits described herein have significant advantages over previous methods and drugs to treat PWS and other diseases/disorders associated with compulsive behaviors such as hyperphagia. For example, the methods, pharmaceutical compositions, and kits described herein are advantageous over previous approaches, because they (1) address hyperphagia through restoring leptin axis; (2) result in weight loss effects that are specific to fat loss, i.e., little or no muscle loss; (3) increase muscle mass; and (4) address compulsive behaviors and anxiety. No other compound has been observed to demonstrate these combined effects.

### Prader-Willi Syndrome

Prader-Willi Syndrome (PWS) is a neurodevelopmental genomic imprinting disorder with lack of expression of genes inherited from the paternal chromosome 15q11-q13 region usually from paternal 15q11-q13 deletions (about 60%) or maternal uniparental disomy 15 or both 15 s from the mother (about 35%). Prader-Willi Syndrome affects approximately 1 in 10,000 to 1 in 25,000 newborns (Killeen, Principles of Molecular Pathology 2004). There are more than 400,000 people who live with Prader-Willi Syndrome around the world (Tweed, AOL Health, September 2009). It is traditionally characterized by hypotonia, short stature, hyperphagia, obesity, behavioral issues (specifically Obsessive-Compulsive Disorder (OCD)-like behaviors), small hands and feet, hypogonadism, and mild intellectual disability (Killeen, Principles of Molecular Pathology 2004). Like autism, Prader-Willi Syndrome is a spectrum disorder and symptoms can range from mild to severe and may change throughout the person's lifetime. Hyperphagia and obesity with related complications are major causes of morbidity and mortality in PWS. In early childhood the disease manifests with a failure-to-thrive syndrome. In late childhood patients become uncontrollably hungry leading to obesity and exhibit intellectual impairment and severe behavioral problems mainly attributed to hyperphagia. The patients are very difficult to manage and have a significantly shortened life-span with an average age of death of 33 years.

Signs and symptoms of Prader-Willi syndrome can vary among individuals. Symptoms may slowly change over time from childhood to adulthood.

### Infants

Signs and symptoms that may be present from birth include:
Poor muscle tone. A primary sign during infancy is poor muscle tone (hypotonia). Babies may rest with their elbows and knees loosely extended instead of fixed, and they may feel floppy or like rag dolls when they're held.
Distinct facial features. Children may be born with almond-shaped eyes, a narrowing of the head at the temples, a turned-down mouth and a thin upper lip.
Poor sucking reflex. Infants may have a poor sucking reflex due to decreased muscle tone. Poor sucking makes feeding difficult and can result in failure to thrive.
Generally poor responsiveness. A baby may seem unusually tired, respond poorly to stimulation, have a hard time waking up or have a weak cry.
Underdeveloped genitals. Males may have a small penis and scrotum. The testicles may be small or not descended from the abdomen into the scrotum (cryptorchidism). In females, the clitoris and labia may be small.

### Early Childhood to Adulthood

Other features of Prader-Willi syndrome appear during early childhood and remain throughout life, requiring careful management. These features may include:
Food craving and weight gain. A classic sign of Prader-Willi syndrome is a constant craving for food, resulting in rapid weight gain, starting around age 2 years. Constant hunger leads to eating often and consuming large portions. Unusual food-seeking behaviors, such as hoarding food, or eating frozen food or even garbage, may develop.
Underdeveloped sex organs. A condition called hypogonadism occurs when sex organs (testes in men and ovaries in women) produce little or no sex hormones. The reduced levels result in underdeveloped sex organs, incomplete or delayed puberty, and in nearly all cases, infertility. Without treatment, women may not start menstruating until their 30s or may never menstruate, and men may not have much facial hair and their voices may never fully deepen.
Poor growth and physical development. Underproduction of growth hormone can result in short adult height, low muscle mass and high body fat. Other endocrine problems may include underproduction of thyroid hormone (hypothyroidism) or central adrenal insufficiency, which prevents the body from responding appropriately during stress or infections.
Cognitive impairment. Mild to moderate intellectual disability, such as issues with thinking, reasoning and problem-solving, is a common feature of the disorder. Even those without significant intellectual disability have some learning disabilities.
Delayed motor development. Toddlers with Prader-Willi syndrome often reach milestones in physical movement—for example, sitting up or walking—later than other children do.
Speech problems. Speech is often delayed. Poor articulation of words may be an ongoing problem into adulthood.
Behavioral problems. Children and adults may at times be stubborn, angry, controlling or manipulative. They may throw temper tantrums, especially when denied food, and may not tolerate changes in routine. They may also develop obsessive-compulsive or repetitive behaviors, or both. Other mental health disorders, such as anxiety and skin picking, may develop.
Sleep disorders. Children and adults with Prader-Willi syndrome may have sleep disorders, including disruptions of the normal sleep cycle and a condition in which breathing pauses during sleep (sleep apnea). These disorders can result in excessive daytime sleepiness and worsen behavior problems.

### Other Signs and Symptoms of PWS

Other signs and symptoms include small hands and feet, curvature of the spine (scoliosis), hip problems, reduced saliva flow, nearsightedness and other vision problems, problems regulating body temperature, a high pain tolerance, or a lack of pigment (hypopigmentation) causing hair, eyes and skin to be pale.

A review of genetics, clinical findings and laboratory testing, clinical and behavioral assessments and summary of updated health-related information addressing the importance of early PWS diagnosis and treatment can be found in Butler et al., (2019) Current Pediatric Reviews 15:207-244, the content of which is incorporated by reference to its entity. Traditionally, Prader-Willi syndrome was diagnosed by clinical presentation. Currently, the syndrome is diagnosed through genetic testing; testing is recommended for newborns with pronounced hypotonia. Early diagnosis of Prader-Willi Syndrome allows for early intervention. The mainstay of diagnosis is genetic testing, specifically DNA-based methylation testing to detect the absence of the paternally contributed Prader-Willi syndrome/Angelman syndrome (PWS/AS) region on chromosome 15q11-q13. Such testing detects over 97% of cases. For example, a chromosome analysis with fluorescence in situ hybridization (FISH) will identify the typical 15q11-q13 deletion but preferably a DNA chromosomal microarray analysis should be performed using both Copy Number Variant (CNV) and single nucleotide poly-morphism (SNP) probes to identify not only the size of the 15q11-q13 deletion (typical and atypical) but helpful for determining the maternal disomy 15 subtype status by identification of Loss of Heterozygosity (LOH) of chromosome 15 gene alleles. Methylation-specific testing is important to confirm the diagnosis of PWS in all individuals, but especially those who are too young to manifest sufficient features to make the diagnosis on clinical grounds or in those individuals who have atypical findings (Buiting et al., Nat. Genet. 9 (4): 395-400, 1995). Droplet Digital PCR can be applied to PWS by identifying small deletions as well as for gene expression studies. It can also identify whether mosaicism exists in an individual with PWS by examining the methylation status or gene expression patterns. There is evidence that some individuals with PWS may have a mixture of normal and abnormal PWS methylation or gene expression patterns, which may be helpful for medical care and diagnosis, specifically in those with PWS and maternal disomy 15. More laboratory and/or clinical tests can be found in Butler et al., 2019.

Another diagnostic tool includes a 13-item informant measure, the Hyperphagia Questionnaire or a variation thereof. This test was developed and administered to the parents of 153 persons with PWS, 4 to 51 years of age. The intelligence quotients, genetic subtypes of PWS, and BMIs offspring were obtained, as were measures of their non-food problem behaviors. Factor analyses with varimax rotation produced three statistically and conceptually robust factors that accounted for 59% of the variance: hyperphagic behaviors, drive, and severity. Hyperphagic behavior increased with age, whereas drive remained stable, and severity dipped in older adults. Hyperphagic drive and severity were positively correlated with non-food behavior problems, and hyperphagic drive differentiated the 36% of participants with extreme obesity from those who had overweight/obese (48%) or healthy (16%) BMI classifications. The Hyperphagia Questionnaire is a valuable tool for relating the neurobiology of hyperphagia to in vivo food-seeking behavior and for examining the psychological and developmental correlates of hyperphagia in PWS. See Dykens et al., (2007) Obesity 15:1816-1826). Additional objective measures may include measurements of body weight and/or body composition and subjective measures may include measurements of caregiver impression, anxiety instrument-PADQ, and/or foodsafe zone score.

Clinical symptoms of PWS may include, e.g., hyperphagia (may begin in early childhood or later), lack of satiety, etc. Clinical signs may include, e.g., Mild prenatal growth retardation-Birth weight, length and body mass index (BMI) may be reduced by 15% compared to unaffected siblings; Hypotonia-Prenatal hypotonia may be evident by reduced fetal movement. Infantile hypotonia (central origin) may be evident by reduced movement, hypoarousal and a weak cry with poor suck and feeding problems; Poor suck and lethargy result in failure to thrive; Global developmental delay and mental deficiency (average IQ-65) occur in nearly all children with PWS in relationship to family background. Studies have shown that verbal comprehension is higher in maternal disomy 15 compared to the 15q11-q13 deletion subtypes, but adaptive function rarely reaches the level predicted by verbal IQ; Dysmorphic craniofacial findings-dolichocephaly, a narrow minimum frontal diameter, strabismus, almond shaped eyes, short upturned nose with thin upper lip and downturned corners of the mouth; Hip dysplasia and subluxation in infants; Thick viscous saliva; Food foraging, hoarding and stealing; Excessive and rapid weight gain leading to obesity; Short stature; Small hands and feet; Narrow hands with straight ulnar border; Kyphosis, lordosis and anterior head tilt (adolescence and adulthood); Hypopigmentation; Genital hypoplasia; Myopia; Enamel hypoplasia, dental caries and dry mucosal membranes, including dental erosion due to bruxism; Skin picking, particularly at a surgical site or in a covered area (e.g., perianal abscess), etc. These phenotypes may be due to hypothalamic dysfunction, which is responsible for hyperphagia, temperature instability, high pain threshold, hypersomnia and multiple endocrine abnormalities including growth hormone and thyroid-stimulating hormone deficiencies, hypogonadism and central adrenal insufficiency (see Angulo et al., (2015) J. Endocrinol. Invest. 38:1249-1263).

In PWS, the term “hyperphagia” encompasses characteristic behaviors around food, such as food obsessions and food-seeking behaviors. Hyperphagia is defined as excessive appetite and is characterized by grossly increased intake of and constant searching for food. It is associated with developmental impairment or behavioral management problems. For example, hyperphagia of PWS is characterized by stealing of food, hiding of food, binge-eating, nighttime eating and eating non-nutritive substances in addition to food items. Much of the morbidity and mortality in PWS is related to the clinical impact of obesity consequent upon the hyperphagia. Hyperphagia itself, independent of obesity, was also important, associated with choking, gastric rupture, and/or respiratory illness. Other syndrome-related factors increased the risk for, and seriousness of, co-morbid illness or accidents. Before hyperphagia becomes prominent, children with PWS may show an unusual interest in food, which can show up as excessive talking about food, such as repeatedly asking for details about what and when they will eat. As hunger increases, the hyperphagic drive to eat can lead them to sneak or steal food, to eat food that is normally considered unacceptable (e.g., food scraps, food from other people's plates, food in the trash, raw food), get up at night to look for food, and take large bites of food and/or eat very fast. In clinical trials that are testing new drugs for their ability to decrease appetite in PWS, a specific questionnaire, the Hyperphagia Questionnaire for Clinical Trials Instrument [HQ-CT; Dykens et al., (2007) Obesity 15:1816-1826 (hereby incorporated by reference), McCandless et al., Diabetes Obes Metab. 2017 December; 19 (12): 1751-1761 (hereby incorporated by reference), and description below), is being widely used to assess changes in hyperphagia behaviors. The instrument provides an assessment of the food-seeking behaviors common among persons with PWS. It has been validated in multiple clinical trials and is designed to evaluate a potential drug candidate's impact on those food-seeking behaviors. The HQ relies on reporting by a single observer who is with the subject every day for a minimum of 4 hours and examines the qualitative and quantitative evidence provide support for use of the 9-item HQ total score over a 2-week recall period. The HQ total score is created by summing the 9 item-level responses (which range from 0 to 4) for a maximum score of 36. The effects of drugs in clinical trials at given timepoints are evaluated by looking at the change in HQ compared to baseline.

In contrast, hyperphagia is not measurable in an obese subject. For example, an obese individual without a hyperphagia disorder would score near 0 (e.g., from 0-2) on a HQ or HQCT evaluation.

Exemplary Hyperphagia Questionnaire for Use in Prader-Willi Syndrome Assessment (See also, “Development of the Hyperphagia Questionnaire for Use in Prader-Willi Syndrome Clinical Trials. Presented at the 2015 ISPOR 20th Annual International Meeting May 16-20, 2015, Philadelphia, PA”, incorporated herein by reference in its entirety).

As was described above, a hallmark of PWS is an incessant feeling of insatiable hunger, regardless of food intake (hyperphagia), and patients typically also have problems with growth and development, intellectual disabilities, maladaptive and compulsive behaviors, and often, severe obesity. The characteristics of PWS have a profound effect on the daily lives of both the individuals with this rare genetic disorder and their caregivers.

Because of the intellectual limitations are commonly associated with PWS, patients may be unable to reliably report the severity of their hyperphagia. As a result, a caregiver-reported measure focused on food-seeking behaviors is used to identify, assess severity of the disease/disorder/syndrome, as well as to evaluate success of treatment, e.g., using the compositions and methods described herein.

The Hyperphagia Questionnaire (HQ) is a caregiver-reported measure of food-seeking behaviors observed among individuals with PWS and utilized within the clinical setting to assess the severity of hyperphagia. The original HQ was subsequently modified to assess hyperphagia in clinical trials and denoted as Hyperphagia Questionnaire for Clinical Trials (HQ-CT). The development and validation of the HQ was conducted per regulatory guidelines as in Example 2. As demonstrated in Example 2, the final HQ was generally deemed clear and easy to understand by the interview participants and all concepts therein were deemed relevant by the interview participants. When asked if any food-related behaviors or concepts were missing, all 6 caregivers reported that the most important concepts were addressed in the HQ.

### Final HQ—Exemplary Questionnaire for Patient Evaluation/Segregation, Evaluation of Treatment Efficacy for Use in Clinical Trials

Exemplary Hyperphagia Questionnaire (HQ) and for Clinical Use
Qualitative and quantitative evidence provide support for use of the 9-item HQ total score for the assessment of food-seeking behaviors for diagnosis and evaluation of PWS.

### Diagnostic Criteria and Monitoring of Clinical Condition/Treatment of PWS

Although diagnosis of PWS can be identified by observation of a subject and clinical diagnostic criteria, e.g., as described above, even in the neonatal period, the molecular techniques, e.g., genetic testing, are also useful to confirm the diagnosis.

The age of diagnosis has fallen significantly in recent years, and the majority of cases are now diagnosed during the first months of life. This development permits earlier introduction of therapies to reduce the morbidity, e.g., by reducing the incidence of or preventing obesity. Earlier intervention not only increases the quality of life for patients but also reduces the burden on the family and caregivers.

### Genetic Testing

PWS arises from the lack of expression of genes on the paternally derived chromosome 15q11-q13. Genes for PWS in this region are physiologically imprinted and silenced on the maternally inherited chromosome. PWS develops if the paternal alleles are defective, missing, or silenced. In about 75% of cases, there is paternal deletion of chromosome 15q11-q13 (type I or II, depending on the proximal breakpoint), maternal uniparental disomy (UPD) in 24%, and imprinting errors in 1% [due in 15% of cases to either a sporadic or inherited microdeletion in the imprinting center, whereas there is a paternal chromosomal translocation in less than 1% of cases.

A variety of methods are useful for confirming the diagnosis and identifying the genetic subtype using peripheral blood lymphocytes, e.g., a method described in American Society of Human Genetics/American College of Medical Genetics Test and Technology Transfer Committee 1996 Diagnostic testing for Prader-Willi and Angelman syndromes. Am J Hum Genet 58:1085-1088). Because imprinted genes demonstrate differential DNA methylation dependent on parental origin, patients with PWS have a maternal-only imprint, because they are lacking a paternal contribution. DNA methylation analysis is the only technique, which can both confirm and reject the diagnosis of PWS, and therefore is typically an initial investigation of choice, e.g., using DNA methylation-specific techniques at the SNURF-SNRPN locus. Parental samples are not required for such an analysis. If DNA methylation analysis shows only a maternal pattern, then PWS is confirmed. Further methods may then be performed to determine the genetic subtype and allow appropriate genetic counseling, in particular the recurrence risk.

Fluorescence in situ hybridization (FISH) analysis is another commonly-used technique and has the advantage of needing only a sample from the proband to detect chromosome 15q11-q13 deletions in PWS. High-resolution chromosome analysis is used to detect interstitial chromosome deletions, and chromosomal translocations or rearrangements may also be detected using this method. Negative FISH or karyotype analysis does not exclude the diagnosis and so if done first may then be followed by a DNA methylation analysis.

If subsequent DNA methylation analysis is positive for PWS, then DNA polymorphism analysis may be performed on the proband and parents to distinguish a maternal UPD from an imprinting defect. Patients with an imprinting defect (ID) warrant further investigation to determine whether an imprinting center deletion is present. Those families with a child with an imprinting center deletion have a recurrence risk of up to 50% if the father of the child is a carrier for the imprinting center deletion. The risk of recurrence in case of chromosomal translocations is evaluated up to 10%. In the other groups, the risk of recurrence is the same as the general population.

Other methods such as methylation-specific multiplex ligation PCR amplification, may be used because and may have the advantage of combining dosing and DNA methylation analysis in one assay, thus distinguishing PWS deletions from UPDs and IDs as well as providing an approximate size of the deletion.

### Genotype-Phenotype Correlations

There are also several biallelic and maternally expressed, paternally imprinted genes throughout the PWS chromosomal region. Their relative under- or overexpression may explain the increasingly recognized genotype-phenotype correlations, such as differences between type I and II deletions, and between deletion and UPD. In particular, hypopigmentation is seen primarily in those with deletion. Patients with UPD have less consistent presence of the characteristic facial phenotype and an increased risk of psychosis but higher verbal intelligence scores and less maladaptive behaviors, compared with patients with deletions. Intellectual ability, academic achievement, and behavioral and psychological problems appear to be worse in subjects with the larger type I deletion than in type II deletion or UPD.

### Prenatal Diagnosis

A prenatal diagnosis is rarely made but could be in cases of reduced fetal movement and polyhydramnios. Genetic testing on samples obtained from chorionic villous sampling and amniocentesis can be performed. FISH analysis can pick up deletions from such tissue, but DNA methylation analysis would be necessary for cases of UPD and IDs.

### Postnatal Diagnosis

There is a marked clinical variability throughout life. The evolving phenotype from birth to adulthood means that the clinical features that may lead to a suspicion of the diagnosis depend on the age of the patient (Table 1). A diagnosis of PWS is particularly helpful during the first months of life and can be considered in all infants with severe and unexplained hypotonia. In addition, the presence of a thin upper lip, almond-shaped eyes, acromicria (short hands and feet), and genital hypoplasia adds to the clinical diagnosis, which can be confirmed by genetic testing. During childhood, a genetic test for PWS is typically not be performed in every obese child with learning disabilities, but a reduced growth velocity, specific dysmorphic features, and history of neonatal hypotonia are strong pointers to initiate testing. Genetic testing can also be considered in adolescents and adults with a less marked phenotype but behavioral and psychological problems in addition to obesity and delayed or incomplete gonadal maturation.

TABLE 1
Indications for DNA testing

| Age at assessment | | Features sufficient to prompt DNA testing |
|---|---|---|
| Birth to 2 yr | | Hypotonia with poor suck |
| 2-6 | yr | Hypotonia with a history of poor suck |
| | | Global developmental delay |
| | | Short stature and/or growth failure associated with |
| | | accelerated weight gain |
| 6-12 | yr | Hypotonia with a history of poor suck (hypotonia |
| | | often persists) |
| | | Global developmental delay |
| | | Excessive eating (hyperphagia, obsession with food) |
| | | with central obesity if uncontrolled |

| 13 yr through | Cognitive impairment, usually mild mental |
|---|---|
| adulthood | retardation |
| | Excessive eating (hyperphagia, obsession with food) |
| | with central obesity if uncontrolled |
| | Hypothalamic hypogonadism and/or typical |
| | behavior problems (including temper tantrums |
| | and obsessive-compulsive features) |

### Differential Diagnosis

Patients with negative testing for PWS may be investigated for other chromosomal deletions and duplications and possible monogenic defects that are associated with PWS or PWS-like features. Other genetic obesity syndromes, such as Bardet-Biedel and fragile X syndrome, associated with cognitive impairment may be indicated. In Bardet-Biedel syndrome, although visual impairment does not usually emerge until 6-8 years of age, other phenotypes such as polydactyly, brachydactyly and high arched palate can assist in early diagnosis.

### Management of PWS and Associated Disorders.

Additional information regarding diagnosis and management of PWS is available, e.g., as described in Goldstone et al., 2008, J. Clin. Endocrinol. Metab. 93:4183-4197, hereby incorporated by reference)

Treatment of PWS involves the management of several co-morbidities that constitute the syndrome and the ongoing assessment for the emergence of associated conditions and complications that evolve over the lifespan of an individual with PWS. As with individuals without PWS, the role of pharmacogenetics and individual drug metabolism rates can influence the effect of the medication on treatment. Replacement for growth hormone, sex steroids, thyroid hormone and glucocorticoids are indicated if and when deficiencies arise. Medications are also used to treat behavioral problems, if behavioral management therapy is unsuccessful. The use of pharmacogenomic testing is becoming more common in clinical practice to check the status of the Cytochrome (CYP) p450 hepatic enzyme system encoded by specific genes required for normal drug metabolism. Alternations in these genes can significantly affect selection of medication types and dosage. Medications can be helpful to manage the obesity-related complications associated with PWS, but there is no medication that has successfully managed the drive for food acquisition. Although, depending on the pharmacogenomics testing, weight gain is noted in those with or without PWS when prescribed certain atypical antipsychotics (e.g., risperidone), if the drug metabolism or neurotransmitter receptor functions are altered. Generally, models to preclinically assess effective PWS treatments may include normal obesity measures in addition to anxiety/compulsive behavior measures which are highly correlated with negative hyperphagic behaviors.

A methionine aminopeptidase 2 (MetAP2) inhibitor, beloranib, was tested for its efficacy, safety, and tolerability for treating PWS (see McCandless et al., (2017) Diabetes Obes Metab. 19 (12): 1751-1761). Participants with PWS (12-65 years) were randomly assigned (1:1:1) to biweekly placebo, 1.8 mg beloranib, or 2.4 mg beloranib injection for 26 weeks at 15 US sites. Coprimary endpoints were the changes in hyperphagia (measured by Hyperphagia Questionnaire for Clinical Trials [HQ-CT]; possible score 0-36) and weight by intention-to-treat. This study was registered on ClinicalTrials.gov as NCT02179151. For the 107 participants, weight change was greater with 1.8 mg (mean difference −8.2%, 95% CI −10.8 to −5.6; p<0.0001) and 2.4 mg beloranib (−9.5%, 95% CI −12.1 to −6.8; p<0.0001). Injection site bruising was the most frequent adverse event with beloranib. Dosing was stopped early due to an imbalance in venous thrombotic events in beloranib-treated participants (two fatal events of pulmonary embolism and two events of deep vein thrombosis) compared to placebo.

Another MetAP2 inhibitor, ZGN-1258, was found to reduce body weight and improve hyperphagia, locomotor activity, and anxiety-like behavior in diet-induced obese (DIO) mouse models of obesity and Prader-Willi Syndrome. The retained efficacy of MetAP2 inhibition in leptin-deficient ob/ob mice and mice with hypothalamic lesions (GTG-induced obesity), as well as extrahypothalamic cFos expression, indicate that extrahypothalmic regions mediate some of the effects of MetAP2 inhibition.

Various approaches to treatment are summarized below.

### Genetic Therapies

| Approach | Mode of Action (MOA) | Advantages | Limitations |
|---|---|---|---|
| Gene activation | Drugs targeting enzymes | Addresses the | Could activate other genes |
| by small | that establish/maintain | underlying cause of | within or outside the PWS |
| molecules | epigenetic marks, | PWS. Would be | region. |
| | activating the PWS genes | applicable to all | There may be critical |
| | on the maternal | subtypes of PWS (del, | “windows” in which |
| | chromosome 15 | UPD, imprinting) | therapy would be |
| | | | effective |
| CRISPR-based | Delivery of a specialized | Addresses the | Delivery likely to be |
| activation | gene/protein to | underlying cause of | challenging. |
| | specifically target and | PW., Applicable to all | Gene therapy is new, so |
| | activate the PWS region | subtypes. | FDA approval path may |
| | on the normally silent | Potential for 1x therapy. | be difficult. |
| | maternal chromosome 15 | | Potential for ‘off target’ |
| | | | effects. |
| Oligonucleotide | Use of small pieces DNA | Addresses the | Need for repeat therapy |
| therapy | or RNA to modify gene | underlying cause of | Oligonucleotide must be |
| | expression | PWS Dosage may be | brain penetrant and will |
| | | better titrated than other | probably need to target |
| | | gene therapy | many cells |
| | | approaches | |
| | | Delivery may be more | |
| | | readily accomplished | |
| AAV-Based Gene | Delivery of specialized | Addresses the underlying | Delivery and targeting |
| Activation | molecules to activate PWS | cause of PWS. Applicable | likely to be challenging. |
| | genes | to all subtypes. | |
| Epigenome Editing | designer epigenome | Addresses the underlying | Development and delivery |
| | modifiers | cause of PWS. Applicable | of epigenetic modifiers is at |
| | | to all subtypes. | early stage; targeting and |
| | | | delivery will be challenging |

### Hyperphagia/Obesity Drugs

| Approach | MOA | Advantages | Limitations |
|---|---|---|---|
| Carbetocin (oxytocin | Binds to oxytocin | Potential to address an | Not yet FDA approved, may |
| analog) | receptors, regulates | underlying deficiency in | act differently than |
| | trust, emotions, | PWS, Oxytocin analog may | oxytocin, Long term safety |
| | appetite | be more specific to oxytocin | and efficacy in PWS |
| | | receptors, potentially less | population not yet known |
| | | side effects than oxytocin | |
| Diazoxide | May affect leptin | New formulation of FDA | Long term safety and |
| Controlled Release | pathways | approved drug Well | efficacy in PWS population |
| (DCCR) | | defined safety profile - | not yet know |
| | | has been used for decades | |
| | | in infants and children | |
| | | with hyperinsulinemia | |
| Oxytocin | Binds to oxytocin | Potential to address an | Complex biology, likely to |
| | receptors, regulates | underlying deficiency in | be complex effects There |
| | trust, emotions, | PWS, may address | may be specific therapeutic |
| | appetite | behavior and | windows depending on |
| | | appetite, FDA approved | age/stage Studies in other |
| | | for other purposes | disorders (autism) have |
| | | | been mixed thus far |
| Tesomet | A combination of | Proposed to act by | Some cv side effects |
| | tesofensine and | reducing appetite, | Not yet FDA |
| | metoprolol to | decreasing food craving | approved Long term safety |
| | reduce appetite | and increasing fat | not yet known |
| | and induce weight | utilization | |
| | loss | Impressive Phase 2 data | |
| | | demonstrating near | |
| | | complete reversal of | |
| | | hyperphagia | |
| Triple reuptake | Hyperphagia | Expected to reduce appetite | Not yet FDA approved. Long |
| inhibitor (CSTI500) | | | term safety not yet known. |
| Cannabidivarin - | Works through | May address both appetite | Previous cannabinoid |
| CBDV | endocannabinoid | and behavior | approaches has severe side |
| | system | | effects |
| | | | Not yet FDA approved Long |
| | | | term safety not yet known |
| CBD | Hyperphagia and | Defined formulation of | Previous cannabinoid |
| | behaviors | CBD. Potential to address | approaches has severe side |
| | | both hyperphagia and | effects |
| | | anxiety | Not yet FDA approved. Long |
| | | | term safety not yet known |
| Peripheral | Decrease activity of | CB1R induced weight loss | Previous cannabinoid |
| Endocannabinoid | endocannabinoid | in a small PWS study but | approaches has severe side |
| (CB1R blocker) | system to decrease | had side effects; ‘peripheral’ | effects |
| | appetite | version is expected to have | Not yet known if targeting |
| | | decreased side effects | this system peripherally will |
| | | | induce weight loss in PWS |
| Aardvark 101 | Gut peptide | Potential to reduce appetite | Not yet FDA approved. Long |
| | stimulator | and obesity. Gut restricted. | term safety not yet known. |
| GLP Receptor | Suppresses appetite | FDA approved May | May slow gut motility, which |
| Agonists (exenatide, | | improve glucose | can be problematic in PWS, |
| liraglutide, etc) | | metabolism, increase satiety | small studies do not show |
| | | | significant weight loss |
| GDD3898 | Reduce excess body | Topical compound to | May not address hunger |
| | fat | reduce excess body fat | |

### Other

| Approach | MOA | Advantages | Limitations |
|---|---|---|---|
| Pitolisant | Increases brain | Approved drug | Does not address |
| | histamine | for narcolepsy | hyperphagia or obesity |

Leptin is a hormone that is produced by adipose tissue and acts through leptin receptors on a variety of central nervous system neurons to ultimately regulate energy homeostasis. Children with PWS have excess serum leptin commensurate with their excess fat mass, but their behavior paradoxically recapitulates a starvation response. Children carrying mutations in the genes encoding leptin or its receptor also become severely hyperphagic and obese. However, these children typically have increased appetites even as infants, leading to body weights far above the 99th percentile by 1.5 years of age, in contrast to infants with PWS. Leptin levels and leptin resistance are correlated to anxiety in T2DM (Cerna et al., (2019) Psychiatry Clin Neurosci. 2019, 73 (12): 745-753) and women (Lawson et al., (2012) Clinical Endocrinology 76:520-525). Leptin has been considered as a therapeutic target for anxiety and depression (Ge et al., (2018) Cell Death and Disease 9:1096)—this has been attributed to extra-hypothalamic alterations in the hippocampus and prefrontal cortex. Lep receptor (LepR) is widely expressed outside hypothalamus including many target tissues for PWS (Scott et al. (2009) J Comp Neurol. 2009, 514 (5): 518-532). Leptin insensitivity is a feature of Magel2 knockout mice, while Magel2 is one of the major genes affected by PWS and the best model of the disease (Pravdivyi et al., (2015) Human Molecular Genetics, 24 (15): 4276-4283).

Compounds described herein may be used to treat PWS or other diseases or disorders with an optional additional agent, such as anti-anxiety agents. Such additional agents may include, e.g., escitalopram, fluoxetine, fluvoxamine, paroxetine, sertraline, duloxetine, venlafaxine, alprazolam, chlordiazepoxide, clonazepam, diazepam, lorazepam, oxazepam, clomipramine, doxepine, imipramine, phenelzine, hydroxyzine, or buspirone. Examples of other agents useful for treatment, together with the compounds described herein, may include antipsychotics, selective serotonin reuptake inhibitors (SSRIs), naltrexone, topiramate, fenfluramine, rimonabant, stimulants, N-acetyle cysteine, or other agents described in Bonnot et al. Eur J Pediatr. 2016; 175 (1): 9-18.

### Concomitant Medications

Concomitant medications (a.k.a., conmeds) are other prescription medications, over-the-counter (OTC) drugs or dietary supplements that a study participant takes in addition to the drug under investigation in a clinical trial. Provided that the conmeds, e.g., those listed below, do not adversely interact with the study medication, such conmeds may be used in combination therapy for treatment of a diseases or disorders treated by the methods described herein (e.g., PWS and associated disorders). A conmed analysis also serves to identify issues to be considered for contraindication, e.g., for inclusion on a label. The list below describes exemplary and common conmeds.

### Anxiety/Antidepressant Medications

Fluoxetine hydrochloride (PROZAC®)
Amitriptyline
Clonidine hydrochloride (off-label)
Wellbutrin™ (Bupropion, Zyban™) (anti-depressant/anxiety, smoking cessation, seasonal affective disorder,

### Sleep Medications/Sleep Related/Stimulants

Melatonin modified-release
Methylphenidate hydrochloride
Methylphenidate hydrochloride extended-release
Dexamfetamine sulphate
Modafinil

### Anti-Psychotic Medications for Behavior or Psychiatric Disorders/Mood Stabilizers

Risperidone
Aripiprazole
Ziprasidone
Olanzapine
Quetiapine
Haloperidol
Lithium Carbonate

### Anti-Convulsant/Anti-Epileptic Medications for Mood Stabilizing or Behavior

Valproic Acid/Sodium Valproate
Lamotrigine
Topirimate

### Benzodiazepines—Fast Acting Anxiety Reducing Sedatives

Lorazepam (Ativan)

### Growth Hormone

Somatropin
Genotropin
Omnitrope
Norditropin FlexPro

In some embodiments, compounds described herein may be used to alleviate at least one symptom (e.g., anxiety, compulsive behavior, etc.) of a subject having PWS or other diseases or disorders described herein. For example, the effectiveness of the treatment using the compounds described herein may be measured by methods such as Hyperphagia Questionnaire (HQ), Hospital Anxiety and Depression Scale (HADS), Hyperphagia Questionnaire for Clinical Trials (HQ-CT), other psychometric evaluation algorithms, or analyses described in Dykens et al., Assessment of hyperphagia in Prader-Willi syndrome. Obesity (Silver Spring). 2007; 15 (7): 1816-1826; FDA Guidance for industry. Patient-reported outcome measures: use in medical product development to support labeling claims. 2009. available at World Wide Web site at fda.gov/downloads/Drugs/Guidances/UCM193282.pdf; Russell et al., The assessment of food-related problems in individuals with Prader-Willi syndrome. Br J Clin Psychol. 2003; 42 (Pt 4): 379-392; Cronbach, Coefficient alpha and the internal structure of tests. Psychometrika. 1951; 16:294-334; Cohen, Statistical power analysis for the behavioral sciences. New Jersey: Lawrence Erlbaum Associates, Inc; 1988.

### Binge Eating Disorder (BED)

Binge eating disorder (BED) is a severe, life-threatening, and treatable eating disorder characterized by recurrent episodes of eating large quantities of food (often very quickly and to the point of discomfort); a feeling of a loss of control during the binge; experiencing shame, distress or guilt afterwards; and not regularly using unhealthy compensatory measures (e.g., purging) to counter the binge eating. It is the most common eating disorder in the United States.

BED is an eating disorder formally recognized in the DSM-5.

An episode of binge eating may be characterized by both of the following:
Eating, in a discrete period of time (e.g., within any 2-hour period), an amount of food that is definitely larger than what most people would eat in a similar period of time under similar circumstances.
A sense of lack of control over eating during the episode (e.g., a feeling that one cannot stop eating or control what or how much one is eating).

Metabolic binge eating episodes may be associated with three (or more) of the following: eating much more rapidly than normal, eating until feeling uncomfortably full, eating large amounts of food when not feeling physically hungry, eating alone because of feeling embarrassed by how much one is eating, feeling disgusted with oneself, depressed, or very guilty afterward, etc. Marked distress regarding binge eating is present. The binge eating occurs, on average, at least once a week for 3 months. The binge eating is not associated with the recurrent use of inappropriate compensatory behaviors (e.g., purging) as in bulimia nervosa and does not occur exclusively during the course of bulimia nervosa or anorexia nervosa.

Emotional and behavioral symptoms of BED may include: evidence of binge eating (disappearance of large amounts of food in short periods of time or lots of empty wrappers and containers indicating consumption of large amounts of food), being uncomfortable eating around others, any new practice with food or fad diets, including cutting out entire food groups (no sugar, no carbs, no dairy, vegetarianism/veganism), fear of eating in public or with others, stealing or hoarding food in strange places, creating lifestyle schedules or rituals to make time for binge sessions, withdrawal from usual friends and activities, frequent diets, extreme concern with body weight and shape, frequent checking in the mirror for perceived flaws in appearance, having secret recurring episodes of binge eating (eating in a discrete period of time an amount of food that is much larger than most individuals would eat under similar circumstances), feeling lack of control over ability to stop eating, disruption in normal eating behaviors, including eating throughout the day with no planned mealtimes, skipping meals or taking small portions of food at regular meals, engaging in sporadic fasting or repetitive dieting, developing food rituals (e.g., eating only a particular food or food group (e.g., condiments), excessive chewing, and not allowing foods to touch), eating alone out of embarrassment at the quantity of food being eaten, feelings of disgust, depression, or guilt after overeating, fluctuations in weight, feelings of low down, stomach cramps, other non-specific gastrointestinal complaints (constipation, acid reflux, etc.), difficulties concentrating, etc.

### Autism

Celastrol (celastrol, a polymorph thereof, or an analogue thereof) is useful to administer to autism subjects for the purpose of reducing the behavior, signs, or symptoms thereof.

The term autism was changed to autism spectrum disorder (ASD) in 2013 by the American Psychiatric Association. ASD is now an umbrella term that covers the following conditions: autistic disorder, pervasive developmental disorder—not otherwise specified (PDD-NOS), and asperger syndrome. People with ASD have trouble with social interactions and with interpreting and using non-verbal and verbal communication in social contexts. Individuals with ASD may also have the following difficulties: inflexible interests, insistence on sameness in environment or routine, repetitive motor and sensory behaviors, like flapping arms or rocking, increased or decreased reactions to sensory stimuli. How well someone with ASD can function in day-to-day life depends on the severity of their symptoms. Many of those with ASD can have delayed or absence of language development, intellectual disabilities, poor motor coordination and attention weaknesses.

Some causes of ASD that are supported by research include genetic and some environmental factors. Specific genetic causes can only be identified in 10% to 20% of cases. These cases include specific genetic syndromes associated with ASD and rare changes in the genetic code. Risk factors include older parental age, low birth weight, prematurity and maternal use of valproic acid or thalidomide during pregnancy, among others.

Some children show signs of autism spectrum disorder in early infancy, such as reduced eye contact, lack of response to their name or indifference to caregivers. Other children may develop normally for the first few months or years of life, but then suddenly become withdrawn or aggressive or lose language skills they've already acquired. Signs usually are seen by age 2 years. Each child with autism spectrum disorder is likely to have a unique pattern of behavior and level of severity—from low functioning to high functioning. Some children with autism spectrum disorder have difficulty learning, and some have signs of lower than normal intelligence. Other children with the disorder have normal to high intelligence—they learn quickly, yet have trouble communicating and applying what they know in everyday life and adjusting to social situations. Because of the unique mixture of symptoms in each child, severity can sometimes be difficult to determine. It's generally based on the level of impairments and how they impact the ability to function.

Several diagnostic instruments are available. Two exemplary tools are commonly used: (1) the Autism Diagnostic Interview-Revised (ADI-R), a semi-structured parent interview, and (2) the Autism Diagnostic Observation Schedule (ADOS), which uses observation and interaction with the child. The Childhood Autism Rating Scale (CARS) is used widely in clinical environments to assess severity of autism based on observation of children. The Diagnostic interview for social and communication disorders (DISCO) may also be used.

Medications may be used to treat ASD symptoms that interfere with integrating a child into home or school when behavioral treatment fails. They may also be used for associated health problems, such as ADHD or anxiety. More than half of US children diagnosed with ASD are prescribed psychoactive drugs or anticonvulsants, with the most common drug classes being antidepressants, stimulants, and antipsychotics. The atypical antipsychotic drugs risperidone and aripiprazole are FDA-approved for treating associated aggressive and self-injurious behaviors. However, their side effects must be weighed against their potential benefits, and people with autism may respond atypically. Side effects, for example, may include weight gain, tiredness, drooling, and aggression. SSRI antidepressants, such as fluoxetine and fluvoxamine, have been shown to be effective in reducing repetitive and ritualistic behaviors, while the stimulant medication methylphenidate is beneficial for some children with co-morbid inattentiveness or hyperactivity.

### Compulsive Behavior

Many mental diseases or disorders, as described herein, may lead to compulsive behaviors. For example, symptoms for both Prader-Willi Syndrome (PWS) and eating disorders (such as binge eating disorder or BED) may include obsessive-compulsive behaviors. Cytoplasmic FMRP-interacting protein 2 (Cyfip2) as a major genetic factor underlying BED and concomitant compulsive-like behaviors in mice. The involved CYFIP2 gene is a homolog of CYFIP1—one of four paternally-deleted genes in patients with the more severe Type I Prader-Willi Syndrome (PWS). Cyfip1 haploinsufficiency (+/−) mice showed increased compulsive-like behavior and induced genetic background-dependent, sex-dependent, and parent-of-origin-dependent effects on palatable food (PF) consumption. CYFIP1 expression could have relevance for neurodevelopmental and neuropsychiatric disorders (see Babbs et al. (2019) G3 (Genes|Genomes|Genetics), 9 (9): 3009-3022).

Obsessive-compulsive disorder (OCD) and autism spectrum disorders (ASD) are two exemplary serious and debilitating psychiatric conditions and each constitutes a significant public health concern, particularly in children. Both of these conditions are highlighted by the repeated expression of meaningless behaviors. Individuals with OCD often show checking, frequent hand washing, and counting. Children with ASDs also engage in repetitive tapping, arm or hand flapping, and rocking. These behaviors can vary widely in intensity and frequency of expression. More intense forms of repetitive behaviors can even result in injury (e.g. excessive grooming, hand washing, and self-stimulation). Treatment options for repetitive behaviors in OCD and ASDs are somewhat limited and there is great interest in developing more effective therapies for each condition. Numerous animal models for evaluating compulsive-like behaviors have been developed over the past three decades. Perhaps the animal models with the greatest validity and ease of use are the marble burying test and the nestlet shredding test (Angoa-Perez et al., (2013) J. Vis. Exp. 82: e50978). Both tests take advantage of the fact that the target behaviors occur spontaneously in mice. In an exemplary marble burying test, 20 marbles are arrayed on the surface of clean bedding. The number of marbles buried in a 30 min session is scored by investigators blind to the treatment or status of the subjects. In an exemplary nestlet shredding test, a nestlet comprised of pulped cotton fiber is pre-weighed and placed on top of cage bedding and the amount of the nestlet remaining intact after a 30 min test session is determined. In addition, nest building may also be used as a common assay for behavioral phenotyping of mouse models with (potential) features of psychiatric disorders.

### Pharmaceutical Compositions

The agents described above, can be formulated into pharmaceutical compositions suitable for use in the present methods. Such compositions include the active agent (e.g., a celastrol compound, a celastrol polymorph (e.g., Form III of Celastrol) together with a pharmaceutically acceptable carrier, excipient or diluent.

In some cases, a pharmaceutical composition includes the active agent (e.g., a celastrol compound, a celastrol polymorph (e.g., Form III of Celastrol) thereof and a combination with one or more pharmaceutically acceptable excipients.

In some cases the pharmaceutical composition containing a celastrol compound, a celastrol polymorph (e.g., Form III of Celastrol), or a celastrol analogue, are administered orally and exhibit a higher bioavailability compared to Celastrol. The oral bioavailability may be at least 1%, 2%, 3%, 4%, 5%, 10%, 15%, 20%, 30%, 40%, 50%, 60%, 80%, 90%, or 95% or higher compared to Celastrol. Furthermore, the oral bioavailability may be at least 1%, 2%, 3%, 4%, 5%, 7%, 10%, 15%, 20%, 25%, 30%, 35%, 40%, 45%, 50%, 60%, 70%, 80%, 90%, or 95% higher compared to intravenous bioavailability for a celastrol compound, a celastrol polymorph (e.g., Form III of Celastrol), or a celastrol analogue, and/or at least 1%, 2%, 3%, 4%, 5%, 7%, 10%, 15%, 20%, 25%, 30%, 35%, 40%, 45%, 50%, 60%, 70%, 80%, 90%, or 95% the level of bioavailability when the celastrol compound, celastrol polymorph (e.g., Form III of Celastrol), or celastrol analogue is administered intravenously.

Pharmaceutical compositions provided herein include compositions wherein the active ingredient is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., induce weight loss. Determination of a therapeutically effective amount of compounds is well within the capabilities of those skilled in the art, especially in light of the detailed disclosure herein.

Pharmaceutically acceptable salts can be prepared by reaction of a free acid or base forms of a compound describes above with a stoichiometric amount of the appropriate be or acid in water, in an organic solvent, or mixture of the two. Lists of suitable salts are found in Remington's Pharmaceutical Sciences, 20th ed., Lippincott Williams & Wilkins, Baltimore, MD, 2000, p. 704; and Handbook of Pharmaceutical salts; Properties, Selection, and Use, P. Heinrich Stahl and Camille G. Wermuth, Eds., Wiley-VCH, Weinheim, 2002.

The agent can also be a pharmaceutically acceptable prodrug of any of the compounds describes above. Prodrugs are compounds that, when metabolized in vivo, undergo conversion to compounds having the desired pharmacological activity. Prodrugs can be prepared by replacing appropriate functionalities present in the compounds describes above with “pro-moieties” as described, for examples, in H. Bundgaar, Design of Prodrugs (1985). Examples of prodrugs include ester, ether or amide derivatives of the compound described above. For further discussion of prodrugs see Rautio, J. et al. Nat. Rev. Drug Disc. 7:255-270, 2008.

For preparing pharmaceutical compositions from comprising compounds disclosed herein, pharmaceutically acceptable carriers can be either solid or liquid. Solid form preparations include powders, tablets, pills, capsules, cachets, suppositories, and dispersible granules. A solid carrier can be one or more substances, that may also act as diluents, flavoring agents, binders, preservatives, tablet disintegrating agents, or an encapsulating material.

In powders, the carrier is a finely divided solid in a mixture with the finely divided active component (e.g. a celastrol compound, a celastrol polymorph, or a celastrol analogue provided herein). In tablets, the active component is mixed with the carrier having the necessary binding properties in suitable proportions and compacted in the shape and size desired. The powders and tablets preferably contain from 5% to 70%, 80%, 85%, 90%, 95%, or 100% of the active compound.

If an excipient is used, suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragees cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for salts disclosed herein are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. Pharmaceutical admixtures suitable for use in in various embodiments disclosed herein are well-known to those of skill in the art and are described, for example, in Pharmaceutical Sciences (17th Ed., Mack Pub. Co., Easton, PA) and WO 96/05309, the teachings of both of which are hereby incorporated by reference.

Aqueous solutions suitable for oral use can be prepared by dissolving the active salt (e.g. compounds described herein, including embodiments, and examples) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, cyclodextrin, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

### Oral Dosage Forms

Oral dosage forms include liquid-filled hard capsules or tablets as well as oral liquids in the form of solutions or suspensions.

In some cases, children cannot swallow large tablets and capsules and are unable to accept formulations that taste bitter or otherwise unpalatable to them. Exemplary clinically desirable pediatric dosage forms involve a solid dosage form, e.g., a capsule or tablet, or an orally dissolvable formulation that is tasteless. Other pediatric formulation include orally disintegrating dosage forms, multi-particulate formulations in the form of powders/sprinkles, mini-tablets, and chewable tablets.

Capsules: Capsules are solid dosage forms in which the drug is enclosed within either a hard or soft soluble container or shell. The shells are typically formed from gelatin; however, they also may be made from starch or other suitable substances. Capsules can contain powder, liquid, suspension, mixture, or colloidal drug (compound or composition) inside the shell.

Lozenges: Lozenges are solid preparations, which are intended to dissolve or disintegrate slowly in the mouth. They contain one or more medicaments, typically in a flavored, sweetened base. They can be prepared by molding (gelatin and/or fused sucrose or sorbitol base) or by compression of sugar based tablets. They contain an active ingredient, e.g., celastrol compound, for systemic absorption after swallowing. Examples include chewable gummy gel lozenge and a hard candy, e.g., a lollipop.

Pastilles: The term pastille is often used for a subclass of lozenges, e.g., molded lozenges. Although some references make no distinction among lozenges, pastilles, and troches, traditionally pastilles were soft lozenges containing medicament in a transparent glycerinated gelatin base or a base of acacia, sucrose, and water; they usually were flavored and colored to match the flavor.

Troches: A troche is a discoid-shaped solid containing the medicinal agent in a flavored base.

Molded tablets: Molded tablets are prepared from mixtures of medicinal substances and a diluent usually consisting of lactose and powdered sucrose in varying proportions. The powders are dampened with solutions containing alcohol. The dampened powders are pressed into molds, removed, and allowed to dry.

The compounds disclosed herein can be administered alone or can be co-administered to the patient. Co-administration is meant to include simultaneous or sequential administration of the compounds individually or in combination (more than one compound). Thus, the preparations can also be combined, when desired, with other active substances (e.g. to induce weight loss).

The compounds can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The salts of compounds disclosed herein can also be administered by injection, that is, intravenously, intramuscularly, intracutaneously, subcutaneously, intraduodenally, or intraperitoneally. It is also envisioned that multiple routes of administration (e.g., intramuscular, oral, intranasal) can be used to administer the salts of compounds disclosed herein. Accordingly, also provided are pharmaceutical compositions comprising a pharmaceutically acceptable excipient and one or more salts of a compound or compounds disclosed herein.

The pharmaceutical preparation can be in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Intranasal Dosage Forms

Without wishing to be bound by theory, in some instances, intranasal administration may increase concentration or bioavailability of the celastrol compound, the celastrol polymorph, or the celastrol analogue, or a prodrug thereof, in the brain. Moreover, without wishing to be bound by theory, in some instances an increased concentration or bioavailability of the celastrol compound, the celastrol polymorph, or the celastrol analogue, or a prodrug thereof, in the brain may increase its efficacy in the methods of the disclosure. For example, in some instances, an advantage of intranasal administration could be bypass of the blood-brain barrier and rapid delivery of a compound (e.g., the celastrol compound, the celastrol polymorph, or the celastrol analogue, or a prodrug thereof) from the nasal mucosa to the brain in a noninvasive way for the treatment of psychiatric or neurological condition such as PWS. Thus, without wishing to be bound by theory, in some examples where the compound is administered via intranasal administration, the compound may be administered at a dosage that is lower than when it is administered via another route, e.g., orally or subcutaneously.

Further, without wishing to be bound by theory, in some instances, intranasal administration of compounds of the disclosure leads to faster absorption of the compounds into the bloodstream compared to administration by other routes, e.g., oral administration. Without wishing to be bound by theory, fast availability of compounds of the disclosure in the blood of a subject may be particularly beneficial for the treatment of psychiatric disorders, such as compulsive behavior disorders where a quick relief from symptoms may be desired.

Nasal dosage forms include solutions, suspensions, emulsions, powders, and gel compositions for nasal delivery. Such dosage forms are administered, for example, using any nebulization and/or atomization device. Non-limiting examples of delivery devices include nasal sprays (e.g., aqueous nasal sprays, hydroalcoholic nasal sprays, or nonaqueous-based nasal sprays), dropper bottles, sophisticated spray pumps (e.g. metered dose spray pumps), dry powder nasal sprays, dry-powder inhalers, and aspirators.

In some examples, a celastrol compound, a celastrol polymorph, or a celastrol analogue of the disclosure is suspended or dissolved in an appropriate carrier, e.g., a pharmaceutically acceptable propellant, and administered using a nasal spray. In some examples, a celastrol compound, a celastrol polymorph, or a celastrol analogue of the disclosure is dissolved, suspended or emulsified in a propellant or a mixture of solvent and propellant, e.g., for administration as a nasal spray. In some cases, pharmaceutical compositions formulated for intranasal delivery (e.g., aerosol formulations) contain any acceptable propellant under pressure, such as a pharmaceutically acceptable propellant, as conventionally used in the art. Non-limiting examples of propellants include liquified gases, including halocarbons, e.g., fluorocarbons such as fluorinated chlorinated hydrocarbons, hydrochlorofluorocarbons, and hydrochlorocarbons), hydrocarbons and hydrocarbon ethers.

In some instances, pharmaceutical compositions formulated for intranasal delivery are similar to nasal secretions in that they are generally isotonic and slightly buffered to maintain a pH of about 5.5 to about 6.5, although it is additionally acceptable to use pH values outside of this range. In some examples, antimicrobial agents or preservatives are included in the formulation.

In some examples, pharmaceutical compositions formulated for intranasal delivery also include other components, for example, ethanol, isopropanol, propylene glycol, as well as surfactants or other components such as oils and detergents. These components serve to, for example, stabilize the formulation and/or lubricate valve components.

In some cases, pharmaceutical compositions formulated for intranasal delivery are packaged under pressure and are formulated as an aerosol using solutions, suspensions, emulsions, powders and semisolid preparations. In some examples, a solution aerosol formulation comprises a solution of a celastrol compound, a celastrol polymorph, or a celastrol analogue of the disclosure, in (substantially) pure propellant or as a mixture of propellant and solvent. For example, in some cases, a solvent is used to dissolve the agent and/or retard the evaporation of the propellant. Solvents useful in the pharmaceutical formulations for intranasal delivery include, for example, water, ethanol and glycols. Solvents useful in the pharmaceutical formulations for intranasal delivery also include any combination of suitable solvents, optionally combined with preservatives, antioxidants, and/or other aerosol components.

In some examples, a pharmaceutical composition formulated for intranasal delivery is a dispersion or suspension of a celastrol compound, a celastrol polymorph, or a celastrol analogue of the disclosure. In some examples, a suspension of a celastrol compound, a celastrol polymorph, or a celastrol analogue of the disclosure comprises a celastrol compound, a celastrol polymorph, or a celastrol analogue of the disclosure, and a dispersing agent. Non-limiting examples of dispersing agents useful in the invention include, sorbitan trioleate, oleyl alcohol, oleic acid, lecithin and corn oil. In some instances, a suspension of a celastrol compound, a celastrol polymorph, or a celastrol analogue of the disclosure further includes lubricants, preservatives, antioxidant, and/or other aerosol components. In some examples, a suspension (e.g., a liquid suspension) of celastrol, a polymorph of celastrol, or a celastrol analogue of the disclosure includes an emulsifier or surfactant (e.g., polysorbate 20) and a mucoadhesive or viscosifier (e.g., HPMC or MCC-NaCMC). In some examples, a suspension (e.g., a liquid suspension) of celastrol, a polymorph of celastrol, or a celastrol analogue of the disclosure further comprises a preservative (e.g., benzalkonium chloride).

In some examples, a pharmaceutical composition formulated for intranasal delivery is formulated as an emulsion. Non-limiting examples of an emulsion of a celastrol compound, a celastrol polymorph, or a celastrol analogue of the disclosure include an alcohol (e.g., ethanol), a surfactant, water and a propellant. In some examples, the surfactant used is nonionic, anionic or cationic. In some examples, an emulsion of a celastrol compound, a celastrol polymorph, or a celastrol analogue of the disclosure, also comprises, for example, vegetable oil, glyceryl monostearate and propane.

Another formulation suitable for intranasal administration is a powder comprising the active ingredient (e.g., a celastrol compound, a celastrol polymorph, or a celastrol analogue of the disclosure) and having an average particle from about 0.2 μm to 500 μm.

### Dosage Amounts and Frequency

The quantity of active component in a unit dose preparation may be varied or adjusted, based on per kg of body weight and/or discretion of a physician or medical practitioner. For example, administration is carried out in fixed doses, e.g., 0.5 mg, 1 mg, 2 mg, 4 mg, 8 mg, which are taken twice weekly or once weekly. The composition can, if desired, also contain other compatible therapeutic agents. Multiple unit doses may be administered within a 24 hour time period. Doses may be administered orally but other routes of administration may also be used depending on the severity of the disease/disorder of the patient.

Various implementations include the oral administration of a compound that is disclosed herein. In some embodiments, the compound is administered at a dose of from about 0.05 to about 100 mg/kg, from about 0.1 to about 0.5 mg/kg, from about 0.1 to about 1 mg/kg, from about 0.1 to about 5 mg/kg, from about 0.1 to about 10 mg/kg, from about 0.1 to about 25 mg/kg, from about 1 to about 5 mg/kg, from about 1 to about 25 mg/kg, from about 5 to about 25 mg/kg, from about 10 to about 25 mg/kg, from about 10 to about 50 mg/kg, from about 25 to about 50 mg/kg, from about 25 to about 75 mg/kg, or from about 50 to about 100 mg/kg. In certain embodiments, the compound is administered at a dose of about 0.1, 0.15, 0.2, 0.25, 0.3, 0.35, 0.4, 0.45, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, 20, 25, 30, 35, 40, 45, 50, 60, 70, 80, 90, or 100 mg/kg. Doses may be administered, e.g., 1, 2, 3, 4, 5, 6, 7, 8, 9, 10, 11, 12, 13, 14, or 15 or more times per day or per week. For example, a compound may be administered once, twice, or three times per day. In some embodiments, the compound is administered before (e.g., about 1, 2, 3, 4, 5, or 6 hours before) or with a meal. Non-limiting examples of methods for converting doses from animals such as mice to human equivalent doses are known in the art. See, e.g., U.S. Food and Drug Administration Center for Drug Evaluation and Research (CDER) (2005) Guidance For Industry: Estimating the Maximum Safe Starting Dose in Initial Clinical Trials for Therapeutics in Adult Healthy Volunteers (available from www.fda.gov/downloads/drugs/guidances/ucm078932.pdf). For example, a mouse dose in mg/kg may be converted to a human equivalent dose (assuming a 60 kg human) based on body surface area by multiplying the mouse dose by 0.08.

In some embodiments, the compounds described herein is administered to a subject in a dose of about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg/kg, or more (and all sub-values and sub-ranges there between, including endpoints). In some embodiments, the compounds described herein is administered to a subject in a dose range of from about 0.1 mg/kg to about 10 mg/kg, from about 0.1 mg/kg to about 5 mg/kg, from about 0.1 mg/kg to about 4 mg/kg, from about 0.1 mg/kg to about 3 mg/kg, from about 0.1 mg/kg to about 2 mg/kg, from about 0.1 mg/kg to about 1 mg/kg, from about 0.5 mg/kg to about 10 mg/kg, from about 0.5 mg/kg to about 5 mg/kg, from about 0.5 mg/kg to about 4 mg/kg, from about 0.5 mg/kg to about 3 mg/kg, from about 0.5 mg/kg to about 2 mg/kg, from about 0.5 mg/kg to about 1 mg/kg, from about 1 mg/kg to about 10 mg/kg, from about 1 mg/kg to about 5 mg/kg, from about 1 mg/kg to about 4 mg/kg, from about 1 mg/kg to about 3 mg/kg, from about 1 mg/kg to about 2 mg/kg, from about 2 mg/kg to about 10 mg/kg, from about 2 mg/kg to about 5 mg/kg, from about 2 mg/kg to about 4 mg/kg, from about 2 mg/kg to about 3 mg/kg, from about 3 mg/kg to about 10 mg/kg, from about 3 mg/kg to about 5 mg/kg, from about 3 mg/kg to about 4 mg/kg, from about 4 mg/kg to about 10 mg/kg, from about 4 mg/kg to about 5 mg/kg, or from about 5 mg/kg to about 10 mg/kg (and all sub-values and sub-ranges there between, including endpoints). A dosage for human may be calculated from a dosage for animals. For example, 0.2 mg/kg for mice corresponds roughly to 1.5 mg/kg for humans. In some embodiments, the compounds described herein is administered to a human subject in a dose range of from about 0.5 mg/kg to about 5 mg/kg, from about 0.5 mg/kg to about 4 mg/kg, from about 0.5 mg/kg to about 3 mg/kg, from about 0.5 mg/kg to about 2 mg/kg, from about 0.5 mg/kg to about 1 mg/kg, from about 1 mg/kg to about 5 mg/kg, from about 1 mg/kg to about 4 mg/kg, from about 1 mg/kg to about 3 mg/kg, from about 1 mg/kg to about 2 mg/kg, from about 2 mg/kg to about 5 mg/kg, from about 2 mg/kg to about 4 mg/kg, from about 2 mg/kg to about 3 mg/kg, from about 3 mg/kg to about 5 mg/kg, from about 3 mg/kg to about 4 mg/kg, or from about 4 mg/kg to about 5 mg/kg (and all sub-values and sub-ranges there between, including endpoints).

Some compounds may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The compositions may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. The disease may be a primary cause for a weight gain disease and/or disorder. The disease may be a caused by a primary weight gain disorder and/or disorder. Other therapeutic regimens or agents can be used in conjunction with the methods and compounds disclosed herein. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In embodiments, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The ratio between toxicity and therapeutic effect for a particular compound is its therapeutic index and can be expressed as the ratio between LD50 (the amount of compound lethal in 50% of the population) and ED50 (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED50 with little or no toxicity. The dosage may vary within this range depending upon the dosage form employed and the route of administration utilized. See, e.g. Fingl et al., In: The Pharmacological Basis of Therapeutics, Ch. 1, p. 1, 1975. The exact formulation, route of administration and dosage can be chosen by the individual physician in view of the patient's condition and the particular method in which the compound is used.

### Kits

In one aspect, provided herein is a kit comprising the pharmaceutical compositions described herein, and instructions for their use. In some embodiments, the kit may be used for an oral administration of the pharmaceutical compositions described herein, for example, such that the kit may further include an applicator for oral administration. In some embodiments, the kit may be used for intraperitoneal administration of the compositions described herein. In some embodiments, the kit may be used for an intranasal administration of the pharmaceutical compositions described herein, for example, such that the kit may further include an applicator for intranasal administration.

### Therapeutic Compounds

Further provided is a celastrol compound, a celastrol polymorph, or a celastrol analogue, that imparts properties for increased or substantially increased oral bioavailability.

A celastrol compound, a celastrol polymorph, or a celastrol analogue of the disclosure may have greater or less solubility in water, an aqueous solution and/or a physiological solution than the Celastrol obtainable from commercial sources or isolated from plants. For example, a celastrol compound, a celastrol polymorph, or a celastrol analogue of the disclosure may have a solubility within ranges from about 0.001 μM to about 150 μM, from 0.01 μM to about 100 μM, from 0.1 μM to about 100 μM, from 1 μM to about 100 μM, from 10 μM to about 100 μM, from 1 μM to about 50 μM, from 10 μM to about 50 μM, from 10 μM to about 80 μM, from 10 μM to about 25 μM, from 25 μM to about 50 μM, from 50 μM to about 100 μM, from 50 μM to about 75 μM, from 25 μM to about 75 μM, or a solubility that is at least about 0.1, 1, 5, 10, 20, 30, 40, 50, 60, 70, 80, 90, or 100 μM, or a solubility that is less than about at least about 0.1, 1, 5, 10, 20, 30, 40, 50, 60, 70, 80, 90, or 100 μM in an aqueous solution (such as phosphate buffered saline (PBS), e.g., at a pH of about 7, 7.1, 7.2, 7.3, 7.4, 7.5, or 7-8).

A celastrol compound, a celastrol polymorph, or a celastrol analogue of the disclosure may have increased or substantially increased stability or half-life in water, aqueous solution or physiological solution. For instance, a celastrol compound, a celastrol polymorph, or a celastrol analogue of the disclosure may have substantially increased stability or resistance in various pH conditions ranging from 2 to 8 in upper or middle gastrointestinal (GI), or digestive tracts.

A celastrol compound, a celastrol polymorph, or a celastrol analogue of the disclosure may impart increased or substantially increased uptake when administered to a subject. For instance, the compounds may have substantially improved permeability across biological membranes. The compounds may exhibit suitable balance between hydrophobicity (lipophilicity) and hydrophilicity by local ionic charges.

Methods, compositions, and kits of the disclosure comprise at least one compound having oral efficacy for treating a disease or disorder described herein.

Methods, compositions, and kits of the disclosure comprise at least one compound having intraperitoneal efficacy for treating a disease or disorder described herein.

### Crystalline Compounds

In some embodiments, the celastrol polymorph (i.e., a polymorph of celastrol) is crystalline Form III, which is characterized by an x-ray powder diffraction pattern comprising peaks at 2θ angles (±0.2) of 9.51°, 14.81°, and 16.84°. In some embodiments, the crystalline Form III is characterized by an X-ray powder diffraction pattern comprising one or more peaks at 2θ angles (±0.2) selected from the group consisting of 13.60°, 14.65°, 18.74°, and 19.10°.

In some embodiments, the crystalline Form III is characterized by a differential scanning calorimetry (DSC) profile with no endothermic peak and an exothermic peak at about 215° C. (onset).

In some embodiments, the crystalline Form III is characterized by a thermal gravimetric analysis (TGA) profile with about 0.5% weight loss at about the exothermic peak at about 215° C. (onset).

In some embodiments, the crystalline Form III is anhydrous solid form. In some embodiments, the anhydrous Form III crystalline solid form is characterized by an X-ray powder diffraction pattern comprising one or more peaks (e.g., one, two, or three peaks) at 2θ angles (±0.2) selected from the group consisting of 9.51°, 14.81°, and 16.84°. In some embodiments, the anhydrous Form III crystalline solid form is characterized by an X-ray powder diffraction pattern comprising peaks at 2θ angles (±0.2) of 9.51°, 14.81°, and 16.84°. In some embodiments, the anhydrous Form III crystalline solid form is characterized by an X-ray powder diffraction pattern further including one or more peaks (e.g., one, two, three, or four peaks) at 2θ angles (±0.2) selected from the group consisting of 13.60°, 14.65°, 18.74°, and 19.10°.

In some embodiments, the anhydrous Form III crystalline solid form is characterized by a differential scanning calorimetry (DSC) profile with no endothermic peak. In some embodiments, the anhydrous Form III crystalline solid form is characterized by a differential scanning calorimetry (DSC) profile with an exothermic peak at about 215° C. (onset). In some embodiments, the anhydrous Form III crystalline solid form is characterized by a differential scanning calorimetry (DSC) profile with no endothermic peak but with an exothermic peak at about 215° C. (onset).

### Definitions

The abbreviations used herein have their conventional meaning within the chemical and biological arts. The chemical structures and formulae set forth herein are constructed according to the standard rules of chemical valency known in the chemical arts.

As used herein, the term “about” in the context of a numerical value or range means ±10% of the numerical value or range recited or claimed, unless the context requires a more limited range.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

Where substituent groups are specified by their conventional chemical formulae, written from left to right, they equally encompass the chemically identical substituents that would result from writing the structure from right to left, e.g., —CH₂O— is equivalent to —OCH₂—.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to ionic compounds that result from the neutralization reaction of an acid and a base. They are composed of related numbers of cations (positively charged ions) and anions (negative ions) so that the product is electrically neutral (without a net charge). These component ions can be inorganic, such as chloride (Cl⁻), or organic, such as acetate (C₂H₃O₂⁻); and can be monatomic, such as fluoride (F⁻), or polyatomic, such as sulfate (SO₄²⁻).

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S) or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more. In addition, the phrase “substituted with a (n),” as used herein, means the specified group may be substituted with one or more of any or all of the named substituents. For example, where a group, such as an alkyl or heteroaryl group, is “substituted with an unsubstituted C1-C20 alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C1-C10 means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated.

The term “alkylsulfate” by itself or as part of another substitutent, means, unless otherwise stated, an alkyl substituted with a sulfate O(SO₂)O⁻ or salt thereof.

The term “alkylsulfonate” by itself or as part of another substitutent, means, unless otherwise stated, an alkyl substituted with a sulfonate (SO₂)O⁻ or salt thereof.

The term “alkylphosphate” by itself or as part of another substitutent, means, unless otherwise stated, an alkyl substituted with a phosphate PO₄₋⁻ or salt thereof.

The term “cycloalkyl”, by itself or as part of another substituent, means, unless otherwise stated, a monocyclic or polycyclic (e.g. bicyclic or tricyclic) saturated hydrocarbon that consists of hydrogen and carbon atoms arranged in a structure containing a single ring or a multiple rings where all of the carbon-carbon bonds are single bonds. Examples of monocyclic alkyl includes cyclopropyl, cyclobutyl, cyclopentyl, cyclohexyl, and the like, and examples of polycyclic alkyl include norbornyl, adamantyl, and the like.

The term “carbocyclic,” by itself or as part of another substituent, means, unless otherwise stated, a cyclic carbon chain (or carbon), which may be fully saturated, mono- or polyunsaturated and can include di- and multivalent radicals, having the number of carbon atoms designated (i.e., C₁-C₁₀ means one to ten carbons). The carbocycle may have a structure containing a single ring or a multiple rings without limitation. Examples of saturated cyclic alkyl groups include cyclopropyl, cyclobutyl, cyclopentyl, cyclohexyl and the like, and examples of unsaturated carbocyclic groups include cyclopropenyl, cyclobutenyl, cyclopentenyl, cyclohexenyl and the like.

The term “heteroalkyl,” by itself or in combination with another term, means, unless otherwise stated, a stable straight or branched non-cyclic chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si, but not limited thereto, may be placed at any interior position of the heteroalkyl group or at the position at which the alkyl group is attached to the remainder of the molecule. Examples include, but are not limited to:—CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH—CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

The term “heterocyclic,” by itself or in combination with another term, means, unless otherwise stated, a cyclic chain, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the cyclic heteroalkyl group or at the position at which the heterocyclic group is attached to the remainder of the molecule. Examples include, but are not limited to: —CO—, —OCOO—, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

The term “secondary amide” by itself or as part of another substitutent, means, unless otherwise stated, an amide in which the nitrogen atom is directly bonded to two carbon atoms.

The term “tertiary amide” by itself or as part of another substitutent, means, unless otherwise stated, an amide in which the nitrogen atom is directly bonded to three carbon atoms.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds, e.g., chemical compounds such as celastrol, analogues thereof, or polymorphs thereof, are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) or polypeptide is free of the amino acid sequences or nucleic acid sequences that flank it in its naturally-occurring state. Purified also defines a degree of sterility that is safe for administration to a human subject, e.g., lacking infectious or toxic agents.

The terms “treating” or “treatment” refers to any indicia of success in the treatment or amelioration of a psychiatric/mental disease, disorder, or condition characterized by compulsive behaviors described herein as well as a injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat a psychiatric/psychological disease or disorder, which may, in some cases, lead to or be associated weight gain such as obesity.

An “effective amount” is an amount sufficient for a compound to accomplish a stated purpose relative to the absence of the compound (e.g. achieve the effect for which it is administered, treat a disease, reduce enzyme activity, increase enzyme activity, reduce signaling pathway, reduce one or more symptoms of a disease or condition. For example, an effective amount of celastrol, an analogue thereof, or a polymorph thereof, is one that reduces a sign or symptom of PWS, e.g., compulsive eating (hyperphagia).

An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g., a mental disease and/or obesity) means that the disease is caused by (in whole or in part), or a symptom of the disease is caused by (in whole or in part) the substance or substance activity or function. As used herein, what is described as being associated with a disease, if a causative agent, could be a target for treatment of the disease. For example, a disease or disorder associated with hyperphagia (e.g., PWS) is treated with an agent that reduces a sign or symptom of PWS, e.g., weight gain, hyperphagia, compulsive behaviors, and/or anxiety. For example, a disease associated with weight gain such as obesity may be treated with an agent (e.g. compound as described herein) effective for decreasing weight gain.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As defined herein, the term “inhibition”, “inhibit”, “inhibiting” and the like in reference to a protein-inhibitor (e.g. antagonist) interaction means negatively affecting (e.g. decreasing) the level of activity or function of the protein relative to the level of activity or function of the protein in the absence of the inhibitor. In some embodiments inhibition refers to reduction of a disease or symptoms of disease. Thus, inhibition may include, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g. increasing) the activity or function of the protein relative to the activity or function of the protein in the absence of the activator (e.g. compound described herein). Thus, activation may include, at least in part, partially or totally increasing stimulation, increasing or enabling activation, or activating, sensitizing, or up-regulating signal transduction or enzymatic activity or the amount of a harmful mediator/substance decreased in a disease. Activation may include, at least in part, partially or totally increasing stimulation, increasing or enabling activation, or activating, sensitizing, or up-regulating signal transduction or enzymatic activity or the amount of a harmful mediator/substance.

The term “modulator” refers to a composition that increases or decreases the level of a target molecule or the function of a target molecule. In embodiments, a modulator is an anti-inflammatory agent. In embodiments, a modulator is an inhibitor of leptin. In embodiments, a modulator is a leptin ligand.

“Anti-obesity agent” refers to the property of a substance or treatment that reduces weight gain and promotes weight loss. Examples of anti-obesity agents would be Sibutramine, Phentermine, Mazindol, Diethylpropion, Leptin, Orlistat, Beta-3 agonists, and Rimonabant.

The term “obese” is used therein, refers to a patient having a body mass index of greater than 30 kg/m². “Overweight” and “pre-obese”, as used herein, refer to patients having a body mass index of greater than 25 kg/m². “Morbidly obese”, as used herein, refers to a patient having a BMI of greater than 40 mg/m², a BMI of greater than 35 kg/m² in combination with one ore more co-morbidities, a BMI of greater than 30 kg/m² in combination with uncontrollable diabetes, or combinations thereof. Examples of ranges of body mass index (BMI) for the definition of obesity or being “obese” may be found at the Center for Disease Control (see, e.g., World Wide Web sites at cdc.gov/obesity/defining.html and cdc.gov/obesity/childhood/defining.html, last accessed on Oct. 20, 2021) or in “Clinical Guidelines on the Identification, Evaluation, and Treatment of Overweight and Obesity in Adults” from the National Institutes of Health. BMI may be obtained by dividing a subject's weight, e.g., in kilograms (kg) by the square of the subject's height, e.g., in meter (m).

The term “prodrug” refers to a pharmacological substance such as a drug that is administered to a subject in an inactive (or significantly less active) form. Once administered, the prodrug is metabolized in the body (in vivo) into a compound having the desired pharmacological activity.

The terms “patient” “subject” “individual” and the like refer to a living organism who suffers from or is susceptible to a disease or condition, e.g., PWS, that can be treated by administration of a compound or pharmaceutical composition as provided herein. Non-limiting examples include humans, other mammals, bovines, rats, mice, dogs, cats, apes, monkeys, goat, sheep, cows, deer, and other non-mammalian animals. In some embodiments, the subject is a companion animal, such as a dog or a cat. In some embodiments, a patient is human. In some embodiments, the patient is pre-obese, obese or morbidly obese. In certain embodiments, the patient is not pre-obese, obese, or morbidly obese, but was formerly pre-obese, obese, or morbidly obese. Alternatively or in addition, a patient has a genetic disorder or behavioral disorder associated with hyperphagia. In some embodiments, the patient has a mental disease, such as a disease or disorder leading to intellectual impairment and/or behavioral problems, or other diseases or disorders disclosed in this specification. In some embodiments, the patient has been diagnosed with PWS. In some embodiments, the patient has more than one disease or disorder, such as a combination of any disease or disorder described herein. In some embodiments, the patient has obesity and at least one mental disease. In some embodiments, the patient to be treated by the compounds described herein has a disease or disorder comprising as Prader-Willi syndrome, Binge eating disorder (BED), Autism, autism spectrum disorders (ASD), obsessive-compulsory disorder (OCD), or depression.

These examples are not limiting. The terms “subject,” “patient,” “individual,” and the like as used herein are not intended to be limiting and can be generally interchanged. That is, an individual described as a “patient” does not necessarily have a given disease or be under the care of a medical professional, but may be merely seeking or wish to have treatment in the absence of medical advice (such as self-treatment). “Syndrome”, “Disease”, “Disorder, or “condition” refer to a state of being or health status of a patient or subject capable of being treated with a compound, pharmaceutical composition, or method provided herein. In some embodiments, the disease is a disease having an increase in body weight. In some embodiments, the disease is obesity. Obesity may be the primary cause of the disease and/or disorder to be treated or may also by a result of the primary disease and/or disorder. In some embodiments, the patient or subject has a mental disease or disorder. In some embodiments, the patient has a Prader-Willi Syndrome (PWS). In some embodiments, the patient or subject has more than one disease or disorder, such as a combination of any disease or disorder described herein. In some embodiments, the patient or subject has obesity and at least one mental disease.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention. In some embodiments, the pharmaceutically acceptable excipient described herein may include at least one of Solutol®, dimethylacetamide, (DMAc or DMA), etc. A pharmaceutical formulation or composition comprises a pharmaceutically-acceptable excipient, carrier, or vehicle.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, or subcutaneous administration, or the implantation of a slow-release device, e.g., a mini-osmotic pump, to a subject. Administration is by any route, including parenteral and transmucosal (e.g., buccal, sublingual, palatal, gingival, vaginal, or rectal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, etc. By “co-administer” it is meant that a composition described herein is administered at the same time, just prior to, or just after the administration of one or more additional therapies (e.g. anti-obesity agent). The compound can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation, to increase degradation of a prodrug and release of the drug, detectable agent). Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions can also be delivered as nanoparticles.

### EXAMPLES

The following examples illustrate certain specific embodiments of the invention and are not meant to limit the scope of the invention. Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1—Treatment of PWS with Compounds of the Disclosure

### Nestlet Shredding in Diet-Induced Obese (DIO) Mice

The study described below was conducted to investigate the effects of a representative compound of the instant disclosure (Celastrol) on repetitive or compulsive behaviours and/or hyperphagic behaviors. Specifically, nestlet shredding, food intake, and body weights were monitored over the course of the study.

C57BL/6 mice were treated with test compound (Celastrol) or controls as shown in the table below from Day 1 to Day 15. No deaths or abnormal clinical signs were reported in the study. One mouse was excluded from the study because it appeared resistant to nestlet shredding prior to treatment.

### Study Groups for the C57BL/6 DIO Study

| | | | Dose, | | |
|---|---|---|---|---|---|
| Group | Mouse # | Treatment (test article) | mg/kg | Route | n |
| 1 | 1-6 | 5% Solutol/5% | 0 | IP | 6 |
| | | DMAC/90% saline | | | |
| 2 | 7-12 | Belaronib | 0.3 | SC | 6 |
| 3 | 13-18 | Celastrol - ERX 1000 | 0.2 | IP | 6 |

The following materials and methods were used to generate the data described herein.

### Mouse Strain and Age

Male C57BL/6 DIO mice were obtained from Jackson Labs (Bar Harbor, ME). The mice arrived at ages 17 weeks and mice were housed 3 per cage on arrival. Prior to dosing in the study, the mice were singly housed.

### Reagents

Celastrol; lot #20552 (also referred to as ERX-1000) was obtained from Medchem Express (Monmouth Junction, NJ). Beloranib (lot #TI/2011/186) was obtained from Medchem Partners (Lexington, MA). Solutol (Kolliphor) HS 15 (lot #BCCD3924) was obtained from Sigma-Aldrich (St. Louis, MO). Dimethylacetamide (lot #SHBM5528) was obtained from Sigma-Aldrich (St. Louis, MO). Saline (lot #19B1455685) was obtained from VWR Chemicals (Radnor, PA). DMSO (Dimethyl sulfoxide; lot #RNBH4175) was obtained from Sigma-Aldrich (St. Louis, MO).

### Treatment Volumes, Routes and Schedules, Syringe, Needle Size

All treatment volumes were 10 mL/kg. Utilizing the art-recognized nestlet shredding mouse model, the dose was administered by gavage (as capsule dosing is challenging in mice). Celastrol and the vehicle control were administered intraperitoneally using a 30G needle. Beloranib was administered subcutaneously using a 30G needle. All treatments were administered daily in the afternoon at 17:00 h, 1 hour before dark cycle from Day 1 to end of dosing.

### Formulation Preparation

Celastrol was dissolved in a pharmaceutically-acceptable excipient, e.g., 50% Solutol/50% DMAC, and this stock solution aliquoted and stored at −80° C. until treatment day. On the day of treatment, an aliquot of the stock solution was thawed and diluted 1:20 with saline to obtain the desired dosing solution strength in 5% Solutol/5% dimethylacetamide/90% saline. The vehicle control was 5% Solutol/5% dimethylacetamide/90% saline. Beloranib was dissolved in 100% DMSO, and this stock solution was aliquoted and stored at −80° C. until treatment day. On the day of treatment, an aliquot of the stock solution was thawed and diluted 1:10 with deionized (DI) water to obtain the desired dosing solution strength in 10% DMSO.

### Weighing Mice

Mouse body weights were recorded from an Ohaus model CS 200 balance. Mice were weighed on Day 1 to Day 15. Body weight changes are expressed as grams and percent of the initial body weight (on Day 1, when treatments began).

### Food Intake

Daily food intake was calculated based on remaining food weight compared to food weight on the previous day using an Ohaus model CS 200 balance.

### Nestlet Shredding

The nestlet shredding test is an art-recognized animal model for human repetitive, anxiety, and/or compulsive behaviors. In the nestlet shredding test, a nestlet comprised of pulped fiber, e.g., cotton fiber, is preweighed and placed on top of cage bedding and the amount of the nestlet remaining intact after a test session of pre-determined time is evaluated.

Nestlet shredding was determined on day 1, day 6, day 11 and day 15 by placing a fresh nestlet prior to the dark cycle and measuring the weight of the remaining nestlet at 12 and 24 hours on an Ohaus model CS 200 balance. Percent shredding was calculated based on the initial nestlet weight.

### Data Recording and Statistics

Data were compiled using Excel. Data are presented as mean±SEM (Standard Error of the Mean) in the table and figures. Student's T-test was performed in Excel. Graphs were prepared using Prism GraphPad.

### Study Results

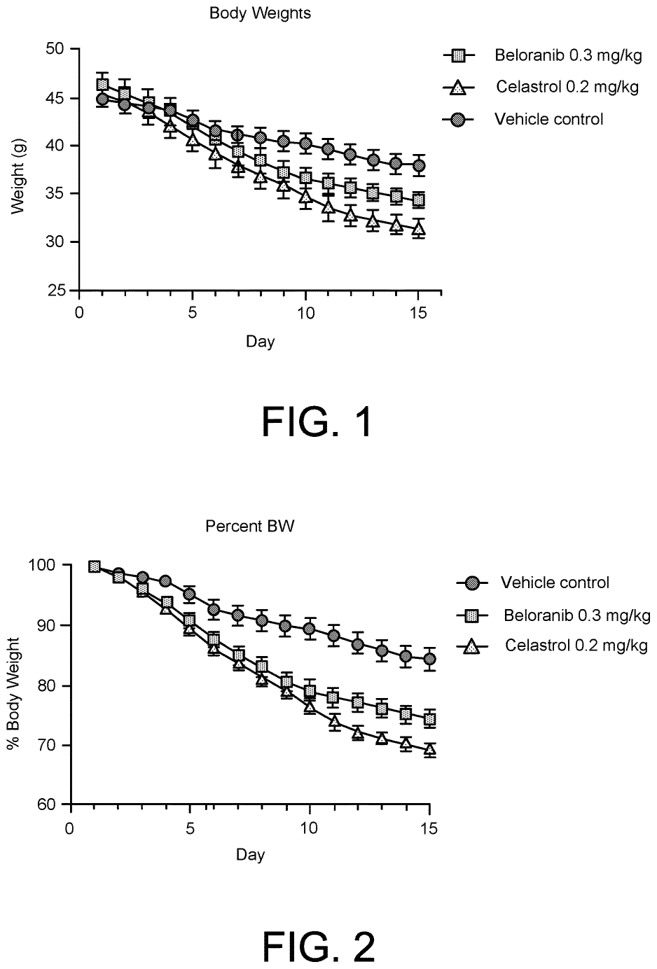

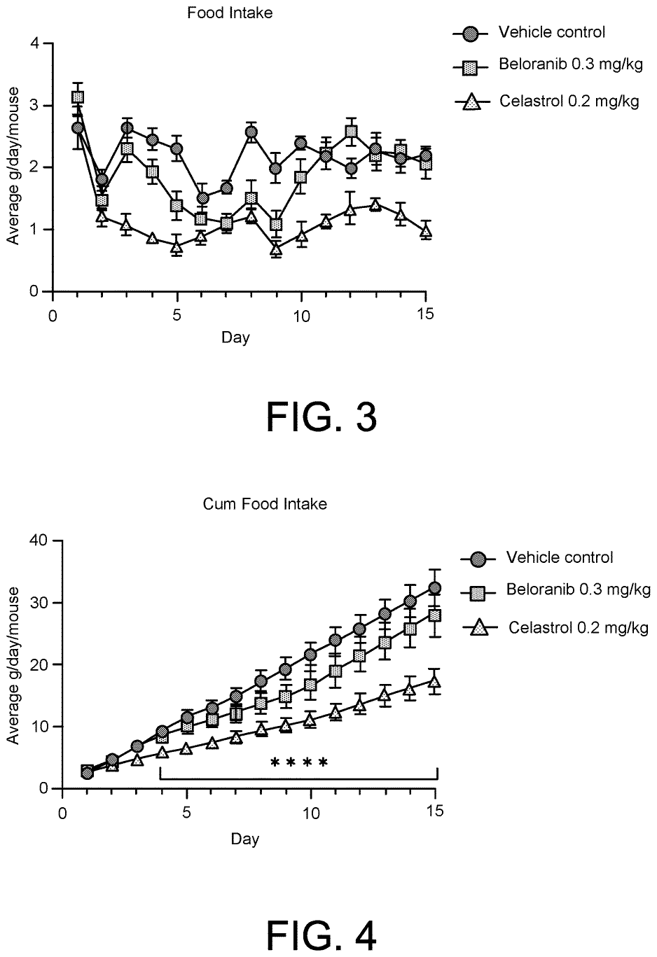

The results of the study are summarized below. Tables 2 and 3 and FIGS. 1 and 2 illustrate the effect of celastrol on body weight in C57BL6 DIO mice. Table 4 and FIG. 3 show the daily food intake over the study. Table 5 and FIG. 4 summarize the nestlet shredding behavior at day 1, 6, 11 and 15 measured after a 12-hour interval after the previous dose on day 0, 5, 10 and 14, respectively.

ol.

TABLE 2
Body weights (in grams) of C57BL6 DIO mice following administration of vehicle, belaronib, or celastrol.

| | | Day | Day | Day | Day | Day | Day | Day | Day | Day | Day | Day | Day | Day | Day | Day |
|---|---|---|---|---|---|---|---|---|---|---|---|---|---|---|---|---|
| | | 1 | 2 | 3 | 4 | 5 | 6 | 7 | 8 | 9 | 10 | 11 | 12 | 13 | 14 | 15 |
| Vehicle | mean | 44.9 | 44.5 | 44.1 | 43.8 | 42.8 | 41.7 | 41.3 | 40.9 | 40.5 | 40.3 | 39.8 | 39.1 | 38.6 | 38.2 | 38.0 |
| control | SEM | 0.4 | 0.4 | 0.5 | 0.6 | 0.8 | 0.9 | 0.9 | 1.0 | 1.1 | 1.0 | 1.1 | 1.0 | 1.1 | 1.0 | 1.1 |
| Belaronib | mean | 46.4 | 45.5 | 44.6 | 43.6 | 42.2 | 40.7 | 39.5 | 38.5 | 37.4 | 36.7 | 36.1 | 35.7 | 35.3 | 34.8 | 34.5 |
| | SEM | 1.3 | 1.5 | 1.5 | 1.4 | 1.5 | 1.5 | 1.5 | 1.3 | 1.2 | 1.1 | 1.0 | 0.9 | 0.9 | 0.8 | 0.8 |
| Celastrol | mean | 45.4 | 44.8 | 43.5 | 42.2 | 40.7 | 39.2 | 38.1 | 37.0 | 36.0 | 34.8 | 33.6 | 32.8 | 32.3 | 31.9 | 31.4 |
| | SEM | 1.3 | 1.3 | 1.3 | 1.3 | 1.4 | 1.5 | 1.4 | 1.5 | 1.4 | 1.3 | 1.4 | 1.1 | 1.1 | 1.1 | 1.0 |

TABLE 3
Body weights (expressed as a percentage of day-1 weight) of C57BL6 DIO
mice following administration of vehicle, belaronib, or celastrol.

| | | Day | Day | Day | Day | Day | Day | Day | Day | Day | Day | Day | Day | Day | Day | Day |
|---|---|---|---|---|---|---|---|---|---|---|---|---|---|---|---|---|
| | | 1 | 2 | 3 | 4 | 5 | 6 | 7 | 8 | 9 | 10 | 11 | 12 | 13 | 14 | 15 |
| Vehicle | mean | 100.0 | 99.0 | 98.2 | 97.5 | 95.3 | 92.8 | 91.8 | 91.0 | 90.1 | 89.5 | 88.4 | 87.1 | 85.9 | 84.9 | 84.5 |
| control | SEM | 0.0 | 0.5 | 0.5 | 0.8 | 1.3 | 1.5 | 1.6 | 1.7 | 1.9 | 1.8 | 1.9 | 1.7 | 1.9 | 1.8 | 2.0 |
| Belaronib | mean | 100.0 | 98.0 | 96.1 | 94.0 | 91.0 | 87.7 | 85.2 | 83.1 | 80.6 | 79.2 | 77.9 | 77.1 | 76.2 | 75.2 | 74.4 |
| | SEM | 0.0 | 0.4 | 0.6 | 0.7 | 1.1 | 1.3 | 1.5 | 1.6 | 1.6 | 1.7 | 1.7 | 1.6 | 1.6 | 1.5 | 1.5 |
| Celastrol | mean | 100.0 | 98.6 | 95.8 | 92.8 | 89.6 | 86.2 | 83.8 | 81.3 | 79.2 | 76.5 | 73.9 | 72.2 | 71.1 | 70.1 | 69.2 |
| | SEM | 0.0 | 0.3 | 0.4 | 0.6 | 1.0 | 1.1 | 1.1 | 1.2 | 1.2 | 1.2 | 1.5 | 1.2 | 1.0 | 1.2 | 1.1 |

TABLE 4
Food intake data (food weights in g/day).

| | | Day | Day | Day | Day | Day | Day | Day | Day | Day | Day | Day | Day | Day | Day | Day |
|---|---|---|---|---|---|---|---|---|---|---|---|---|---|---|---|---|
| | | 1 | 2 | 3 | 4 | 5 | 6 | 7 | 8 | 9 | 10 | 11 | 12 | 13 | 14 | 15 |
| Vehicle | mean | 2.6 | 1.8 | 2.6 | 2.5 | 2.3 | 1.5 | 1.7 | 2.6 | 2.0 | 2.4 | 2.2 | 2.0 | 2.3 | 2.1 | 2.2 |
| control | SEM | 0.3 | 0.2 | 0.2 | 0.2 | 0.2 | 0.2 | 0.1 | 0.2 | 0.2 | 0.1 | 0.2 | 0.2 | 0.3 | 0.2 | 0.2 |
| Belaronib | mean | 3.1 | 1.5 | 2.3 | 1.9 | 1.4 | 1.2 | 1.1 | 1.5 | 1.1 | 1.9 | 2.2 | 2.6 | 2.2 | 2.2 | 2.1 |
| | SEM | 0.3 | 0.2 | 0.2 | 0.2 | 0.2 | 0.2 | 0.1 | 0.3 | 0.2 | 0.3 | 0.3 | 0.2 | 0.3 | 0.2 | 0.2 |
| Celastrol | mean | 2.7 | 1.2 | 1.1 | 0.9 | 0.7 | 0.9 | 1.1 | 1.2 | 0.7 | 0.9 | 1.1 | 1.3 | 1.4 | 1.2 | 1.0 |
| | SEM | 0.1 | 0.1 | 0.2 | 0.1 | 0.2 | 0.1 | 0.1 | 0.1 | 0.1 | 0.2 | 0.1 | 0.3 | 0.1 | 0.2 | 0.2 |

TABLE 5
Nestlet Shredding data (percent of nestlet shredded).

| | | Day 1 | Day 6 | | Day 11 | | Day 15 | |
|---|---|---|---|---|---|---|---|---|
| | | 12 hour | 12 hour | 24 hour | 12 hour | 24 hour | 12 hour | 24 hour |
| Vehicle | mean | 89.4 | 96.9 | 100.0 | 96.3 | 98.1 | 100.0 | 100.0 |
| control | SEM | 6.9 | 3.1 | 0.0 | 3.7 | 1.9 | 0.0 | 0.0 |
| Belaronib | mean | 95.5 | 11.9 | 15.7 | 3.8 | 5.2 | 27.6 | 32.9 |
| | SEM | 4.9 | 10.8 | 10.4 | 1.9 | 2.5 | 7.0 | 10.0 |
| Celastrol | mean | 94.4 | 70.1 | 93.6 | 43.7 | 71.0 | 50.9 | 63.3 |
| | SEM | 5.6 | 15.0 | 6.4 | 19.8 | 14.9 | 18.5 | 20.3 |

FIG. 1 shows the body weight effects over the 14 days of dosing of Beloranib or ERX-1000 (Celastrol). The vehicle group lost body weight from an average of 44.9 g to 38 g. The body weight of mice treated with beloranib decreased, on average, from 46.4 g to 34.5 g and the body weight of mice treated with Celastrol decreased, on average, from 44.8 g to 31.4 g.

FIG. 2 shows the body weight expressed as a percentage of day 1 body weight. The vehicle-treated mice lost 15.5% at day 15. Beloranib treatment led to 25.6% body weight lost (significant from vehicle control; p=0.0023). Celastrol-treated mice lost 30.8% body weight (significant from vehicle control; p=0.00005).

FIG. 3 shows the daily food intake over the study. The food intake of the vehicle group remained fairly constant around 2.2 g/day/mouse. The beloranib group had a reduction of food intake that initiated on day 2-3 and reached a nadir of 1.1 g/day/mouse but then rebounded to be similar to vehicle at day 10-11 of 2.2 g/day/mouse. The Celastrol group displayed a reduction in food intake that initiated on day 2 and remained throughout the course of the 15-day study at an average of 1.0 g/day/mouse (significant from vehicle control).

FIG. 4 shows the cumulative food intake over the study. The food intake of the vehicle group stayed relatively constant per day and averaged a total of 32.6 g for the study. The beloranib group fluctuated and was reduced in the first week of the study but then returned to that of the vehicle control group for an average total of 28.1 g/mouse and was not statistically different from vehicle control. The celastrol group had a significant reduction in food intake that was maintained for the entire study. The average total was 17.4 g/mouse and the difference was significant from vehicle control starting on day 4 onward.

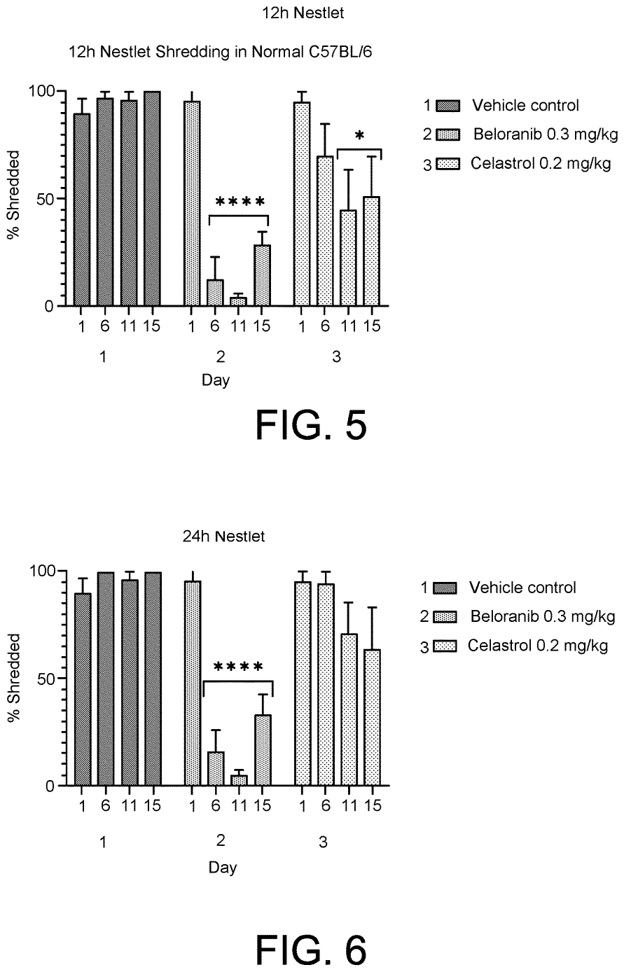

FIG. 5 shows the nestlet shredding behavior at day 1, 6, 11 and 15 measured after a 12-hour interval after the previous dose on day 0, day 5, day 10 and day 14, respectively. The vehicle group demonstrated nearly 100% nestlet shredding at all timepoints. Both treatment groups started on day-1 with essentially 100% shredding behavior. The beloranib group exhibited 11.9, 3.8 and 27.6% shredding on average on day 6, day 11 and day 15 respectively and were all significant from vehicle control. The celastrol group exhibited 70.1, 43.7 and 50.9% shredding on average on day 6, day 11 and day 15 respectively. The shredding results achieved significance from vehicle control on day 11 and day 15.

FIG. 6 shows the nestlet shredding behavior at day 1, 6, 11 and 15 measured after a 24-hour interval after the previous dose on day 0, day 5, day 10 and day 14, respectively. The vehicle group demonstrated nearly 100% nestlet shredding at all timepoints. Both treatment groups started on day −1 with essentially 100% shredding behavior. The beloranib group exhibited sustained inhibition of shredding at 15.7, 5.2 and 32.9% shredding on average on day 6, day 11 and day 15 respectively and were all significant from vehicle. The ERX-1000 (celastrol) group exhibited 93.6, 71.0 and 63.3% shredding on average on day 6, day 11 and day 15 respectively.

Without wishing to be bound by theory, the profile of the nestlet shredding was different between the positive control Beloranib and Celastrol in terms of sustained inhibition of shredding behavior from 12- to 24-hours. In addition, celastrol was found to have a more potent and sustained effect on food intake, where Beloranib's effects on food intake rebounded.

### Nestlet Shredding in Magel2 Mice

The nestlet shredding behavioral effects of celastrol was evaluated in Magel2 knockout mice. Magel2 knockout has been used as a model of Prader-Willi syndrome to assess potential pharmacological treatments effect on behavior (Meziane 2015). Without wishing to be bound by theory, the Magel2 gene is a major causative gene of phenotypes in both Prader-Willi syndrome and autism (Schraff 2013). Further, nestlet shredding has been used to assess anxiety and compulsive behaviors in mouse models (Angoa-Perez 2013 and Dorniger 2020) and has been used to characterize clinically effective treatments for Prader-Willi syndrome (Fagen 2018 and McCandless 2017).

In the study disclosed herein, celastrol at was administered intraperitoneally using a 30G needle at a dose of 0.2 mg/kg to five female Magel2 homozygous mice (−/−) and four male mice heterozygous for Magel2 (−/+). In addition, vehicle was administered to four male mice heterozygous for Magel2 (−/+) for 21 days. No deaths or abnormal clinical signs were reported during the evaluation phase of the study.

### Study Groups for the C57BL/6 Magel2 Study

| | Mouse | Treatment | Dose, | | | |
|---|---|---|---|---|---|---|
| Group | # | (test article) | mg/kg | Schedule | Route | n |
| 1- Vehicle control Magel2(−/+) | 1-4 | 5% Solutol/5% DMAC/ | 0 | QD | IP | 4 |
| | | 90% saline | | | | |
| 2 - Treated Magel2 (−/+) | 5-8 | Celastrol - ERX 1000 | 0.2 | QD | IP | 4 |
| 3 - Treated Magel2 (−/−) | 9-13 | Celastrol - ERX 1000 | 0.2 | QD | IP | 5 |

The following materials and methods were used to generate the data described herein.

### Mice Strain and Age

C57BL/6-Magel2 mice were bred for purpose from cryo recovery by Jackson Labs (Bar Harbor, ME) and arrived at the testing facility at age 8 weeks.

### Reagents

Celastrol, was obtained from Medchem Express (Monmouth Junction, NJ)

Solutol (Kolliphor) HS 15 lot #BCCD3924 was obtained from Sigma-Aldrich (St. Louis, MO)

Dimethylacetamide lot #SHBM5528 was obtained from Sigma-Aldrich (St. Louis, MO)

Saline lot #19B1455685 was obtained from VWR Chemicals (Radnor, PA)

DMSO, lot #RNBH4175, was obtained from Sigma-Aldrich (St. Louis, MO)

### Treatment Volumes, Routes and Schedules, Syringe, Needle Size

All treatment volumes were 10 mL/kg. Utilizing the art-recognized nestlet shredding mouse model, the dose was administered by gavage (as capsule dosing is challenging in mice). Celastrol and the vehicle control were administered intraperitoneally using a 30G needle. All treatments were administered daily in the afternoon at 17:00 h, 1 hour before dark cycle from Day 1 to end of dosing.

### Formulation Preparation

Celastrol was dissolved in 50% Solutol/50% DMAC. The resulting stock solution aliquoted and stored at −80° C. until treatment day. On the day of treatment an aliquot of the stock solution was thawed and diluted 1:20 with saline to obtain the desired dosing solution strength in 5%

Solutol/5% dimethylacetamide/90% saline. The vehicle control was 5%

Solutol/5% dimethylacetamide/90% saline.

### Weighing Mice

Mouse body weights were recorded from an Ohaus model CS 200 balance. Mice were weighed daily on Day 1 to Day 21. Body weight changes are expressed as grams and percent of the initial body weight (on Day 1, when treatments began).

### Food Intake

Daily food intake was calculated based on remaining food weight compared to food weight on the previous day using an Ohaus model CS 200 balance.

### Nestlet Shredding

The nestlet shredding test is an art-recognized animal model for human repetitive, anxiety, and/or compulsive behaviors. In the nestlet shredding test, a nestlet comprised of pulped fiber, e.g., cotton fiber, is preweighed and placed on top of cage bedding and the amount of the nestlet remaining intact after a test session of pre-determined time is evaluated.

Nestlet shredding was determined on day 1, day 11, and day 21 by placing a fresh nestlet prior to the dark cycle and measuring the weight of the remaining nestlet at 12 and 24 hours on an Ohaus model CS 200 balance. Percent shredding was calculated based on the initial nestlet weight.

### Data Recording and Statistics

Data were compiled using Excel. Data are presented as mean±SEM in the text and figures. Student's Ttest was performed in Excel. Graphs were prepared using Prism GraphPad.

### Study Results

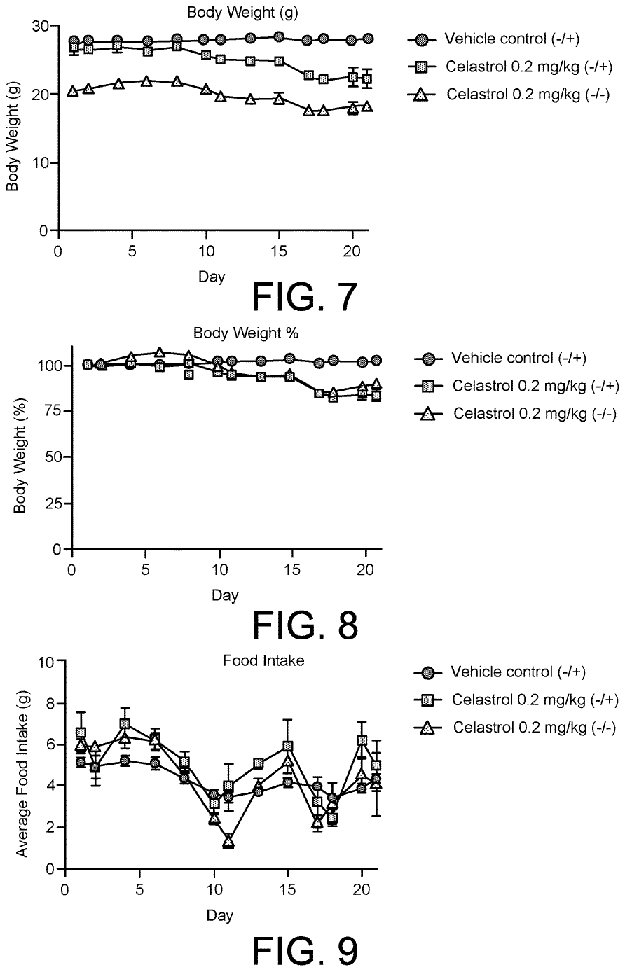

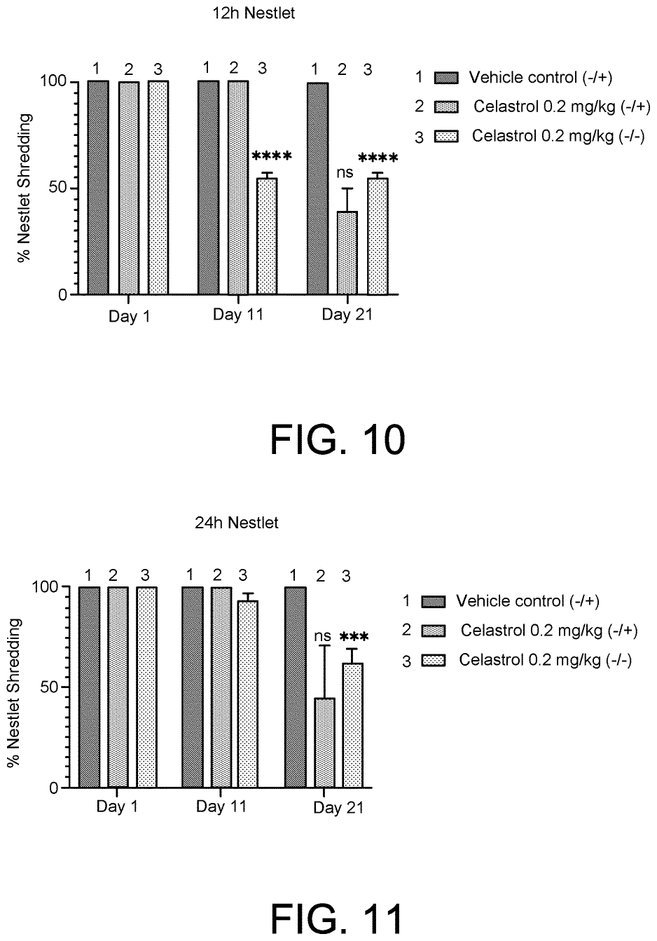

The results of the study are summarized below. Tables 6 and 7 and FIGS. 7 and 8 illustrate the effect of celastrol on body weight in C57BL6 DIO mice. Table 8 and FIG. 9 show the daily food intake over the study. Table 9 and FIG. 10 summarize the nestlet shredding behavior at day 1, 11 and 21 measured after a 12-hour interval after the previous dose on day 0, 10 and 20, respectively. Without wishing to be bound by theory, effects of ceolastrol on nestlet shredding behavior was most pronounced in the homozygous knockout mice, which are the model of Prader-Willi syndrome.

TABLE 6
Body weights (in grams) of C57BL6 Heterozygous control and Magel2
mice following administration of vehicle, or celastrol.

| | | Day | Day | Day | Day | Day | Day | Day | Day | Day | Day | Day | Day | Day |
|---|---|---|---|---|---|---|---|---|---|---|---|---|---|---|
| | | 1 | 2 | 4 | 6 | 8 | 10 | 11 | 13 | 15 | 17 | 18 | 20 | 21 |
| Vehicle | mean | 27.5 | 27.6 | 27.7 | 27.6 | 27.7 | 27.9 | 28.0 | 28.1 | 28.4 | 27.8 | 28.1 | 28.0 | 28.2 |
| control | SEM | 0.5 | 0.4 | 0.5 | 0.5 | 0.5 | 0.4 | 0.4 | 0.4 | 0.5 | 0.5 | 0.4 | 0.4 | 0.4 |
| Celastrol | mean | 26.6 | 26.5 | 26.9 | 26.4 | 26.9 | 25.6 | 25.1 | 25.0 | 25.0 | 22.6 | 22.1 | 22.5 | 22.3 |
| heterozygous | SEM | 0.9 | 0.7 | 0.8 | 0.6 | 0.7 | 0.6 | 0.7 | 0.5 | 0.7 | 0.6 | 0.7 | 1.4 | 1.3 |
| control mice | | | | | | | | | | | | | | |
| Celastrol | mean | 20.5 | 20.8 | 21.7 | 22.0 | 21.8 | 20.7 | 19.6 | 19.2 | 19.5 | 17.5 | 17.6 | 18.3 | 18.4 |
| Magel2 mice | SEM | 0.2 | 0.4 | 0.3 | 0.3 | 0.5 | 0.6 | 0.5 | 0.5 | 0.6 | 0.4 | 0.5 | 0.6 | 0.6 |

TABLE 7
Body weights (expressed as a percentage of day-1 weight) of C57BL6 Heterozygous
control and Magel2 mice following administration of vehicle, or celastrol.

| | | Day | Day | Day | Day | Day | Day | Day | Day | Day | Day | Day | Day | Day |
|---|---|---|---|---|---|---|---|---|---|---|---|---|---|---|
| | | 1 | 2 | 4 | 6 | 8 | 10 | 11 | 13 | 15 | 17 | 18 | 20 | 21 |
| Vehicle | mean | 100.0 | 100.5 | 100.6 | 100.4 | 100.8 | 101.4 | 101.9 | 102.2 | 103.2 | 101.2 | 102.2 | 101.7 | 102.7 |
| control | SEM | 0.0 | 0.5 | 0.8 | 0.8 | 1.1 | 1.1 | 1.2 | 1.3 | 1.2 | 2.1 | 2.0 | 1.5 | 1.4 |
| Celastrol | mean | 100.0 | 99.6 | 101.1 | 99.4 | 101.3 | 96.4 | 94.4 | 94.2 | 93.9 | 85.0 | 83.0 | 84.5 | 83.8 |
| heterozygous | SEM | 0.0 | 1.2 | 1.0 | 1.3 | 1.3 | 2.2 | 2.5 | 2.2 | 1.4 | 0.7 | 0.9 | 3.0 | 3.2 |
| control mice | | | | | | | | | | | | | | |
| Celastrol | mean | 100.0 | 101.2 | 105.6 | 107.1 | 106.2 | 100.9 | 95.6 | 93.5 | 95.0 | 85.3 | 85.6 | 88.8 | 89.6 |
| Magel2 mice | SEM | 0.0 | 0.9 | 0.6 | 0.8 | 1.4 | 1.7 | 1.7 | 1.9 | 2.1 | 1.3 | 1.5 | 2.0 | 2.0 |

TABLE 8
Food intake data (food weights in g/day).

| | | Day | Day | Day | Day | Day | Day | Day | Day | Day | Day | Day | Day | Day | Day |
|---|---|---|---|---|---|---|---|---|---|---|---|---|---|---|---|
| | | −2 | 1 | 2 | 4 | 6 | 8 | 10 | 11 | 13 | 15 | 17 | 18 | 20 | 21 |
| Vehicle | mean | 3.6 | 5.2 | 4.9 | 5.2 | 5.1 | 4.4 | 3.6 | 3.5 | 3.7 | 4.2 | 4.0 | 3.4 | 3.9 | 4.4 |
| control | SEM | 0.5 | 0.2 | 0.6 | 0.2 | 0.3 | 0.3 | 0.2 | 0.3 | 0.2 | 0.2 | 0.1 | 0.2 | 0.2 | 0.2 |
| Celastrol | mean | 5.0 | 6.6 | 4.9 | 7.0 | 6.3 | 5.2 | 3.1 | 3.9 | 5.1 | 5.9 | 3.2 | 2.5 | 6.2 | 5.0 |
| heterozygous | SEM | 0.8 | 1.0 | 0.9 | 0.8 | 0.5 | 0.5 | 0.5 | 0.2 | 0.2 | 1.3 | 1.2 | 0.4 | 0.9 | 1.3 |
| control mice | | | | | | | | | | | | | | | |
| Celastrol | mean | 3.2 | 5.9 | 5.9 | 6.4 | 6.2 | 4.6 | 2.4 | 1.4 | 4.0 | 5.2 | 2.2 | 3.2 | 4.6 | 11.1 |
| Magel2 mice | SEM | 0.2 | 0.3 | 0.1 | 0.5 | 0.4 | 0.3 | 0.3 | 0.3 | 0.3 | 0.6 | 0.4 | 1.0 | 0.9 | 1.5 |

TABLE 9
Nestlet Shredding data (percent of nestlet shredded).

| | | Day 1 | Day 11 | | Day 21 | |
|---|---|---|---|---|---|---|
| | | 12 hour | 12 hour | 24 hour | 12 hour | 24 hour |
| Vehicle | mean | 100.0 | 100.0 | 100.0 | 96.0 | 100.0 |
| control | SEM | 0.0 | 0.0 | 0.0 | 4.0 | 0.0 |
| Celastrol | mean | 100.0 | 100.0 | 100.0 | 38.3 | 44.6 |
| heterozygous | SEM | 0.0 | 0.0 | 0.0 | 22.8 | 26.1 |
| control mice | | | | | | |
| Celastrol | mean | 100.0 | 54.7 | 93.5 | 54.7 | 62.3 |
| Magel2 mice | SEM | 0.0 | 6.0 | 3.5 | 5.5 | 7.0 |

FIG. 7 shows the body weight effect over the 21 days of dosing in grams. The vehicle group remains relatively weight stable growing from an average of 27.5 g on day 1 to 28.2 on day 21. The body weight of male heterozygous mice decreased from an average of 26.6 g on day 1 to 22.3 g on day 21 and the body weight of female homozygous mice decreased from an average of 20.5 g on day 1 to 18.4 g on day-21.

FIG. 8 shows the body weight effect over the 21 days of dosing in percent of day 1. The vehicle group remains relatively weight stable growing +2.7% from day 1 to day 21. Male heterozygous mice lost 16.2% body weight from day 1 to day 21 and the female homozygous mice lost 10.4% on day-21.

FIG. 9 shows the daily food intake over the study. There was not a significant difference in food intake between the groups. There were some signs from day-11 to day-21 that both treated groups were nibbling food as exhibited by crumbs found in the cage which made an accurate weighing of the food difficult.

FIG. 10 shows the nestlet shredding behavior at day 1, 11, and 21 measured after a 12-hour interval after the previous dose on day 0, 10, and 20. The vehicle group demonstrated nearly 100% nestlet shredding at all timepoints. Both treatment groups also started on day 1 with 100% shredding behavior. The treated heterozygous male showed a decrease in nestlet shredding behavior at day 21 with an average of 38.3% that did not reach statistical significance due to the high variability. The treated homozygous females had a pronounced nestlet shredding behavior effect that began on day 11 at the 12 hour timepoint showing 54.7% shredding that was highly statistically significant (p<0.0003). This behavior persisted through day 21 showing a highly statistically significant effect on shredding 54.7%. The p-value for this measurement was <0.0007.

FIG. 11 shows the nestlet shredding behavior at day 1, 11 and 21 measured after a 24-hour interval after the previous dose on day 0, 10, and 20. The vehicle group demonstrated nearly 100% nestlet shredding at all timepoints. Both treatment groups also started on day 1 with 100% shredding behavior. The treated heterozygous male showed a strong trend toward decrease nestlet shredding behavior at day 21 with an average of 44.6% that did not reach statistical significance due to the high variability. The treated homozygous females had a pronounced nestlet shredding behavior effect through day 21 showing a highly statistically significant effect on shredding (62.3%). The p-value for this measurement was <0.002.

### Example 2—Development of HQ Questionnaire

### Modification of the HQ

The developers of the HQ and additional experts in the development and validation of clinical outcome assessments modified the HQ with the following objectives:
   Limit the concepts of measurement to behaviors that are observable by caregivers (without inference) and that have the potential to change during the course of a clinical trial
   Incorporate industry guidance related to patient- and caregiver-reported outcome measures, including recommendations in the FDA PRO guidance.

### Psychometric Evaluation

The preliminary version of the HQ was administered in the context of a phase 2 clinical trial in patients with PWS to evaluate the performance of each item, develop a scoring algorithm, and evaluate psychometric properties such as reliability and validity.
The phase 2 clinical trial was a single-center, randomized, double-blind, placebo-controlled study involving overweight and obese adult patients with PWS.
The 17 subjects, residents in a group home dedicated to patients with PWS, were randomized to one of three dosing arms, including placebo and two doses of drug.
Data collected during clinic visits at the beginning of the placebo lead-in period (week 1), the beginning of randomized treatment (week 3), and the end of randomized treatment (week 7) were used in the psychometric evaluation.
In addition to the preliminary version of the HQ, the following measures were used in the evaluation:
   Weight in kilograms
   Food-Related Problem Questionnaire (FRPQ): A 16-item measure developed to assess preoccupation with food, satiety impairment, and food-related negative behaviors in patients with PWS.3
   Food intake/behavior daily journal (FIB-DJ): A daily caregiver observations (i.e., counts) diary of several aberrant or food-related observable behaviors, such as stealing food and aggression.

### Exemplary HQ Analyses

Item-response frequencies were tabulated to assess the use and appropriateness of the response scales and to identify possible floor and ceiling effects.
Item-level standard descriptive statistics (mean, standard deviation [SD], median, range, and percentage missing) were computed to assess the distribution of HQ scores across time.
Item-total correlations and inter-item correlations for the HQ were computed to inform the HQ scoring algorithm.
Cronbach's coefficient alpha was computed to investigate the internal consistency of candidate composite HQ scores.
To evaluate construct validity, correlations (Spearman) were conducted among the HQ items, candidate HQ composite(s), weight, FRPQ items and subscales, and weekly counts of FIB-DJ behaviors.
Preliminary estimates of responsiveness were evaluated by computing responsiveness effect size estimates for each HQ item.
Due to small sample sizes, Cohen's general rule of thumb was applied to characterize effect size estimates in change scores (i.e., 0.20, small; 0.50, moderate; 0.80, large).

### Qualitative Interviews

In-depth interviews were conducted with 6 caregivers of children or adults with PWS at a regional conference for a PWS patient support organization. Inclusion criteria for the caregivers were as follows:
   Primary caregiver of an individual with PWS, aged 13 to 65 years
   If PWS patient is an adult, must have a body mass index (BMI) of >30
   If PWS patient is a child, must have a BMI>95th percentile for age and sex, per the National Health and Nutrition Examination Survey (NHANES)
Interviews were conducted using a semistructured interview guide and included both a concept elicitation exercise and cognitive debriefing of an electronic version of the HQ to meet the following objectives:
   Inform any further refinements to facilitate ease of item comprehension and response by caregivers
   Provide additional support for content validity
   Optimize the usability of the electronic data capture device
      HQ modifications
Initial modifications included reducing the recall period to 2 weeks, revising the response scales to match the recall period, and modifying the wording of several items to be more objective and observable (e.g., replaced “to what extent” with “how often,” removed “food-related thoughts” and maintained “food-related talk or behavior”).
Three items were removed, because they did not address observable behaviors or were not expected to change with a reduction in hyperphagia (i.e., child's age of increased interest in food, variability of the preoccupation or interest in food, being clever or fast to obtain food).
Based on the initial modifications of the HQ, a preliminary 10-item version of the HQ was created. Other variations can be made, e.g., by evaluating various parameters as described herein.

### Psychometric Evaluation.

Overall, results of the psychometric evaluation supported the use of a single HQ composite score, as well as the reliability and validity of this measure.
The HQ total score was able to demonstrate improvements in hyperphagia-related behavior between placebo and pooled treatment groups as shown in Table 10.

TABLE 10
Exemplary HQ Item-Level Responsiveness: Changes
From Week 3 to Week 7 by Treatment (N = 17)

| | | Mean (SD) | | | | |
|---|---|---|---|---|---|---|
| | | Placebo | | Treatment | | Effect |
| | HQ Item | (n = 6) | | (n = 11) | | Size |
| 1. | Upset when denied food | −0.17 | (1.2) | 0.09 | (1.4) | 0.22 |
| 2. | Try to bargain or manipulate | −0.17 | (0.4) | 0.18 | (1.7) | 0.85 |
| 3. | Effort required to redirect | 0.50 | (1.0) | −0.27 | (0.8) | −0.74 |
| 4. | Forage through trash for food | 0.33 | (0.8) | −0.27 | (0.9) | −0.74 |
| 5. | Get up at night to food seek | −0.17 | (0.4) | −0.18 | (0.6) | −0.04 |
| 6. | Persistence after being told no | −0.33 | (1.4) | −0.09 | (0.8) | 0.18 |
| | more | | | | | |
| 7. | Time spent talking about food | 0.00 | (1.7) | −0.91 | (1.3) | −0.54 |
| 8. | Try to steal food | 0.33 | (0.5) | −0.36 | (1.1) | −1.35 |
| 9. | Distress when told to stop | 0.00 | (1.1) | 0.36 | (1.2) | −0.33 |
| | food-related talk | | | | | |
| 10. | Interference with daily | −0.33 | (1.0) | −0.09 | (1.0) | 0.23 |
| | activities from food- related | | | | | |
| | talk or behavior | | | | | |

| | | | | | |
|---|---|---|---|---|---|
| Total: 10-item sum | 0.00 | (5.8) | −2.27 | (4.3) | −0.39 |

SD = standard deviation.

### Item Reduction

The content of item 3 (“ . . . once the person started talking about food, how much effort did it take to get him/her to stop talking about food and on to other things?”) was re-evaluated:
   Similar to that of another item (item 6, “ . . . how persistent was the person in asking or looking for food after being told ‘no’ or ‘no more’?”)
   Focused more on interactions between the caregiver and patient than on the patient's behavior per se
Following discussion, developers agreed that removal of this item would not compromise the content validity of the measure. Thus, item 3 of the preliminary 10-item HQ was eliminated, resulting in a 9-item version of the HQ for further evaluation during the qualitative interviews.

### Qualitative Interviews

Characteristics for the 6 caregivers and the individuals under their care are provided in Table 11.

TABLE 11
Exemplary characteristics of Qualitative Interview Participants

| | PWS Caregivers | |
|---|---|---|
| Characteristic | (N = 6) | |
| Caregiver | | |
| Sex, female, n (%) | 4 | (66.7) |
| Relationship to person under care, parent, n | 6 | (100.0) |
| (%) | | |
| Race/ethnicity, white, n (%) | 6 | (100.0) |
| Education, n (%) | | |
| High school | 1 | (16.7) |
| College degree | 2 | (33.3) |
| Postgraduate coursework/advanced | 3 | (50.0) |
| degree | | |
| Person under care | | |
| Age in years, mean (range) | 32.7 | (22.0-44.0) |
| BMI, mean (range) | 34.6 | (30.5-46.6) |
| Living situation (≥50% of the time), n (%) | | |
| Home/private residence | 3 | (50.0) |
| Group home for patients with PWS^{a} | 3 | (50.0) |

^{a}Patients were required to spend time at their parents'/caregivers' homes where the caregivers were directly involved in their care.

The 9 HQ items were generally deemed clear and easy to understand by the interview participants; caregivers also indicated that the 2-week recall period was appropriate and easy to reference.
All concepts were deemed relevant by the interview participants. When asked if any food-related behaviors or concepts were missing, all 6 caregivers reported that the most important concepts were already addressed in the HQ.
Based on the results of the qualitative interviews, minor wording changes were made to several HQ items.
The electronic handheld device was deemed easy to use by all interview participants.

### Example 3—Intranasal Administration of Celastrol

Sprague Dawley rats aged 5-6 weeks and weighing approximately 150-250 g (N=8) were intranassaly administered exemplary liquid formulations containing a celastrol drug substance (comprising crystalline Form III of celastrol) at a dosing volume of 0.1 mL/kg (corresponding to a dose concentration of 2 mg/kg). Additional rats (N=4) were administered a celastrol drug substance (comprising crystalline Form III of celastrol) formulated in 0.01% w/v polysorbate 80, delivered via oral gavage at a dosing volume of 10 mL/kg. The intranasally administered liquid formulations are summarized in Table 12.

TABLE 12
Tested intranasal liquid suspension formulations of celastrol

| Formulation | Formulation Composition (mg/mL) | |
|---|---|---|
| Intranasal A | Celastrol drug substance:Polysorbate | 20:25:0.1:1 |
| | 20:Benzalkonium chloride:Hydroxypropyl | |
| | methylcellulose | |
| Intranasal B | Celastrol drug substance:Polysorbate | 20:25:0.1:18 |
| | 20:Benzalkonium chloride:Combination of | |
| | microcrystalline cellulose and sodium | |
| | carboxymethyl cellulose (MCC-NaCMC) | |

Whole blood samples (~100 μl) were obtained at 5 min, 15 min, 30 min, 1 h, and 4 h or at 5 min, 15 min, 30 min, 1 h, 2 h, 4 h, 8 h, and 24 h after administration and immediately placed back on wet ice. After centrifugation at 6000 rpm for 5 minutes at 4° C., plasma was collected and stored at −80° C. until analysis. The concentration of celastrol in the blood samples was determined by LC-MS. Table 13 summarizes the results of the study.

TABLE 13
Pharmacokinetic parameters

| | | Cₘₐₓ | Tₘₐₓ | AUC₀₋ₗₐₛₜ |
|---|---|---|---|---|
| Formulation | Measured at | (ng/mL) | (h) | (ng*hr/mL) |
| Intranasal A | 5 min, 15 min, 30 min, 1 h, and 4 h | 3.44 | 1.00 | 11.1 |
| Intranasal A | 5 min, 15 min, 30 min, 1 h, and 4 h | 2.22 | 1.00 | 6.32 |
| Intranasal A | 5 min, 15 min, 30 min, 1 h, 2 h, 4 h, 8 h, and 24 h | 4.17 | 1.00 | 19.1 |
| Intranasal A | 5 min, 15 min, 30 min, 1 h, 2 h, 4 h, 8 h, and 24 h | 2.93 | 1.00 | 20.3 |
| Intranasal B | 5 min, 15 min, 30 min, 1 h, and 4 h | 3.44 | 0.500 | 5.16 |
| Intranasal B | 5 min, 15 min, 30 min, 1 h, and 4 h | 4.75 | 1.00 | 11.8 |
| Intranasal B | 5 min, 15 min, 30 min, 1 h, 2 h, 4 h, 8 h, and 24 h | 5.65 | 1.00 | 48.6 |
| Intranasal B | 5 min, 15 min, 30 min, 1 h, 2 h, 4 h, 8 h, and 24 h | 19.6 | 1.00 | 89.7 |
| Oral | 5 min, 15 min, 30 min, 1 h, and 4 h | 1.7 | 4.00 | 3.46 |
| Oral | 5 min, 15 min, 30 min, 1 h, and 4 h | 1.09 | 4.00 | 2.01 |
| Oral | 5 min, 15 min, 30 min, 1 h, 2 h, 4 h, 8 h, and 24 h | 3.57 | 4.00 | 53.9 |
| Oral | 5 min, 15 min, 30 min, 1 h, 2 h, 4 h, 8 h, and 24 h | 2.64 | 8.00 | 42.7 |

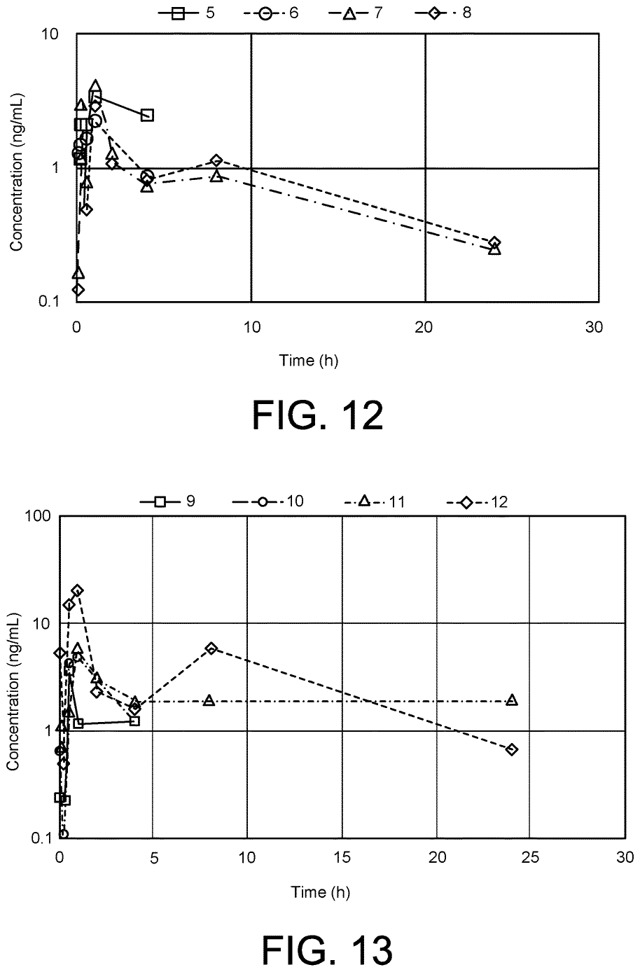

FIG. 12 shows the concentration of celastrol in plasma of male Sprague Dawley rats following intranasal dosing of a liquid intranasal formulation (Intranasal A) at a dose concentration of 2 mg/kg.

FIG. 13 shows the concentration of celastrol in plasma of male Sprague Dawley rats following intranasal dosing of a liquid intranasal formulation (Intranasal B) at a dose concentration of 2 mg/kg.

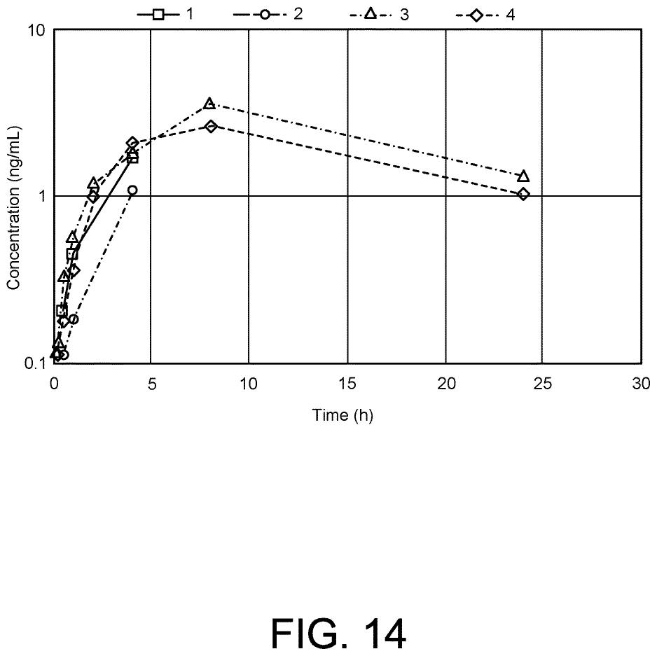

FIG. 14 shows the concentration of celastrol in plasma of male Sprague Dawley rats following oral dosing of celastrol at a dose concentration of 2 mg/kg.

As demonstrated above, intranasal administration using the tested liquid formulations resulted in plasma concentrations of celastrol that were comparable to or higher than plasma concentrations of celastrol administered orally. However, maximum plasma concentrations (Cmax) were achieved faster following the intranasal administration than following oral administration (at about 1 h following the intranasal administration compared to 4 h following oral administration).

### ENUMERATED EMBODIMENTS

Embodiment 1. A method of any one of the aspects disclosed in the Summary.

Embodiment 2. A pharmaceutical composition of any one of the aspects disclosed in the Summary.

Embodiment 3. A kit of any one of the aspects disclosed in the Summary.

Embodiment 4. The pharmaceutical composition of Embodiment 2, wherein the pharmaceutical composition comprises the active ingredient (e.g., celastrol, celastrol polymorph, or celastrol analogue) and a pharmaceutically acceptable excipient.

Embodiment 5. The pharmaceutical composition of Embodiment 2, wherein the pharmaceutical composition comprises the active ingredient (e.g., celastrol, celastrol polymorph, or celastrol analogue) does not comprise a pharmaceutically acceptable excipient.

Embodiment 6. The pharmaceutical composition of Embodiment 2, wherein the pharmaceutical composition comprises the celastrol compound, celastrol polymorph, or celastrol analogue in the form of a powder packaged in a capsule.

Embodiment 7. The kit of Embodiment 3, further comprising a tablet, capsule, or an oral applicator.

Embodiment 8. The kit of Embodiment 3, further comprising celastrol, a polymorph thereof, or an analogue thereof as a powder in a capsule.

Embodiment 9. The kit of Embodiment 3, further comprising instructions for use.

Embodiment 10. The kit of Embodiment 3, further comprising an oral applicator, and instructions for use.

Embodiment 11. The kit of Embodiment 3, further comprising an intranasal applicator, and instructions for use.

Embodiment 12. The kit of Embodiment 3, further comprising guidelines or instructions for administration for the treatment of a disorder or disease comprising Prader-Willi syndrome, Binge eating disorder (BED), Autism, autism spectrum disorders (ASD), obsessive-compulsory disorder (OCD), depression, or combinations thereof.

Embodiment 13. The method, pharmaceutical composition, or kit of any one of the preceding embodiments, wherein the mental disease or disorder or the disease characterized by compulsive behavior comprises Prader-Willi syndrome, Binge eating disorder (BED), Autism, autism spectrum disorder (ASD), obsessive-compulsory disorder (OCD), depression, or combinations thereof.

Embodiment 14. The method, pharmaceutical composition, or kit of any one of the preceding embodiments, wherein the mental disease or disorder or a disease characterized by compulsive behavior comprises hyperphagia.

Embodiment 15. The method, pharmaceutical composition, or kit of any one of the preceding embodiments, wherein the psychiatric disorder is a compulsive behavior disorder.

Embodiment 16. The method, pharmaceutical composition, or kit of any one of the preceding embodiments, wherein the subject is human.

Embodiment 17. The method, pharmaceutical composition, or kit of any one of the preceding embodiments, wherein the subject is a child.

Embodiment 18. The method, pharmaceutical composition, or kit of any one of the preceding embodiments, wherein the subject is a child.

Embodiment 19. The method, pharmaceutical composition, or kit of any one of the preceding embodiments, wherein the subject is 2 years of age or greater than 2 years of age.

Embodiment 20. The method, pharmaceutical composition, or kit of any one of the preceding embodiments, wherein the subject is 12 years of age or greater than 12 years of age.

Embodiment 21. The method, pharmaceutical composition, or kit of any one of the preceding embodiments, wherein the subject is between 2 years of age and 100 years of age, inclusive of the endpoints.

Embodiment 22. The method, pharmaceutical composition, or kit of any one of the preceding embodiments, wherein the subject is between 2 years of age and 90 years of age, inclusive of the endpoints.

Embodiment 23. The method, pharmaceutical composition, or kit of any one of the preceding embodiments, wherein the subject is between 2 years of age and 80 years of age, inclusive of the endpoints.

Embodiment 24. The method, pharmaceutical composition, or kit of any one of the preceding embodiments, wherein the subject is between 2 years of age and 70 years of age, inclusive of the endpoints.

Embodiment 25. The method, pharmaceutical composition, or kit of any one of the preceding embodiments, wherein the subject is between 2 years of age and 60 years of age, inclusive of the endpoints.

Embodiment 26. The method, pharmaceutical composition, or kit of any one of the preceding embodiments, wherein the subject is between 2 years of age and 50 years of age, inclusive of the endpoints.

Embodiment 27. The method, pharmaceutical composition, or kit of any one of the preceding embodiments, wherein the subject is between 2 years of age and 40 years of age, inclusive of the endpoints.

Embodiment 28. The method, pharmaceutical composition, or kit of any one of the preceding embodiments, wherein the subject is between 2 years of age and 25 years of age, inclusive of the endpoints.

Embodiment 29. The method, pharmaceutical composition, or kit of any one of the preceding embodiments, wherein the subject is between 12 years of age and 100 years of age, inclusive of the endpoints.

Embodiment 30. The method, pharmaceutical composition, or kit of any one of the preceding embodiments, wherein the subject is between 12 years of age and 90 years of age, inclusive of the endpoints.

Embodiment 31. The method, pharmaceutical composition, or kit of any one of the preceding embodiments, wherein the subject is between 12 years of age and 80 years of age, inclusive of the endpoints.

Embodiment 32. The method, pharmaceutical composition, or kit of any one of the preceding embodiments, wherein the subject is between 12 years of age and 70 years of age, inclusive of the endpoints.

Embodiment 33. The method, pharmaceutical composition, or kit of any one of the preceding embodiments, wherein the subject is between 12 years of age and 60 years of age, inclusive of the endpoints.

Embodiment 34. The method, pharmaceutical composition, or kit of any one of the preceding embodiments, wherein the subject is between 12 years of age and 50 years of age, inclusive of the endpoints.

Embodiment 35. The method, pharmaceutical composition, or kit of any one of the preceding embodiments, wherein the subject is between 12 years of age and 40 years of age, inclusive of the endpoints.

Embodiment 36. The method, pharmaceutical composition, or kit of any one of the preceding embodiments, wherein the subject is between 12 years of age and 25 years of age inclusive of the endpoints.

Embodiment 37. The method, pharmaceutical composition, or kit of any one of the preceding embodiments, wherein the subject is not obese.

Embodiment 38. The method, pharmaceutical composition, or kit of any one of the preceding embodiments, wherein the subject is pre-obese, obese, or morbidly obese.

Embodiment 39. The method of any one of the preceding embodiments, wherein the celastrol compound, the celastrol polymorph, or the celastrol analogue, is administered orally, in the form of a capsule or tablet.

Embodiment 40. The pharmaceutical composition or kit of any one of the preceding embodiments, wherein the pharmaceutical composition, by itself or as included in the kit, is formulated for administration orally, in the form of a capsule or tablet.

Embodiment 41. The pharmaceutical composition or kit of any one of the preceding embodiments, wherein the pharmaceutical composition, by itself or as included in the kit, is formulated for administration by subcutaneous or intravenous administration. The method of any one of the preceding embodiments, wherein the celastrol compound, the celastrol polymorph, or the celastrol analogue, is administered by subcutaneous or intravenous administration.

Embodiment 42. The method of any one of the preceding embodiments, wherein the composition is in the form of a capsule or tablet and is administered to the subject by oral administration.

Embodiment 43. The method of any one of the preceding embodiments, wherein the composition is administered by oral, subcutaneous, or intravenous administration.

Embodiment 44. The method of any one of the preceding embodiments, wherein the composition is administered by oral administration.

Embodiment 45. The method of any one of the preceding embodiments, wherein the composition is administered by subcutaneous administration.

Embodiment 46. The method of any one of the preceding embodiments, wherein the celastrol compound, the celastrol polymorph, or the celastrol analogue, or a pharmaceutical composition comprising the celastrol compound, the celastrol polymorph, or the celastrol analogue is administered by intranasal administration.

Embodiment 47. The method of any one of the preceding embodiments, wherein the celastrol compound, the celastrol polymorph, or the celastrol analogue, or a pharmaceutical composition comprising the celastrol compound, the celastrol polymorph, or the celastrol analogue is administered by transdermal administration.

Embodiment 48. The method of any one of the preceding embodiments, wherein the pharmaceutical composition, by itself or as included in the kit, is administered to the subject using oral administration, intravenous administration, subcutaneous administration, intranasal administration, transdermal administration, topical administration, parenteral administration, intraperitoneal administration, intramuscular administration, intrathecal administration, intralesional administration, intracranial administration, intraocular administration, intracardiac administration, intravitreal administration, intraosseous administration, intracerebral administration, intraarterial administration, intraarticular administration, intradermal administration, transmucosal administration, sublingual administration, enteral administration, sublabial administration, insufflation administration, suppository administration, or inhaled administration.

Embodiment 49. The pharmaceutical composition or kit of any one of the preceding embodiments, wherein the pharmaceutical composition, by itself or as included in the kit, is formulated for administration to the subject using oral administration, intravenous administration, subcutaneous administration, topical administration, parenteral administration, intraperitoneal administration, intramuscular administration, intrathecal administration, intralesional administration, intracranial administration, intraocular administration, intracardiac administration, intravitreal administration, intraosseous administration, intracerebral administration, intraarterial administration, intraarticular administration, intradermal administration, transmucosal administration, sublingual administration, enteral administration, sublabial administration, insufflation administration, suppository administration, or inhaled administration.

Embodiment 50. The method of any one of the preceding embodiments, wherein the celastrol compound, the celastrol polymorph, or the celastrol analogue, or a pharmaceutical composition comprising the celastrol compound, the celastrol polymorph, or the celastrol analogue is administered to the subject using intranasal administration, and is formulated as a intranasal dosage form.

Embodiment 51. The method of any one of the preceding embodiments, wherein the nasal doage form comprises a solution, suspension, emulsion, powder, or gel composition for intranasal delivery.

Embodiment 52. The method of any one of the preceding embodiments, wherein the intranasal dosage form is administered via a nasal spray, a dropper bottle, or a metered dose spray pump.

Embodiment 53. The method of any one of the preceding embodiments, wherein the nasal spray is an aqueous nasal spray, a hydroalcoholic nasal spray, or a nonaqueous-based nasal spray.

Embodiment 54. The method of any one of the preceding embodiments, wherein the intranasal dosage form is administered via a dry-powder nasal spray, dry-powder inhaler, or aspirator.

Embodiment 55. The method any of the preceding embodiments, wherein the intranasal dosage form is a liquid suspension

Embodiment 56. The method of any of the preceding embodiments, wherein the pharmaceutical composition is a liquid suspension.

Embodiment 57. The pharmaceutical composition of any of the preceding embodiments, wherein the pharmaceutical composition is a liquid suspension for intranasal administration.

Embodiment 58. The method or pharmaceutical composition of any of the preceding embodiments, wherein the liquid suspension comprises a celastrol compound, a celastrol polymorph, or a celastrol analogue and an emulsifier or surfactant.

Embodiment 59. The method or pharmaceutical composition of any of the preceding embodiments, wherein the liquid suspension comprises a preservative.

Embodiment 60. The method or pharmaceutical composition of any of the preceding embodiments, wherein the liquid suspension comprises
(i) a pharmaceutical agent, wherein the pharmaceutical agent is a celastrol compound, a celastrol polymorph, or a celastrol analogue;
(ii) an emulsifier or surfactant; and
(iii) a mucoadhesive or viscosifier;
wherein the pharmaceutical composition is in the form of a liquid suspension.

Embodiment 61. The method or pharmaceutical composition of any of the preceding embodiments, wherein the liquid suspension comprises
(i) a pharmaceutical agent, wherein the pharmaceutical agent is a celastrol compound, a celastrol polymorph, or a celastrol analogue;
(ii) a polysorbate; and
(iii) a cellulose;
wherein the pharmaceutical composition is in the form of a liquid suspension.

Embodiment 62. The method or pharmaceutical composition of any of the preceding embodiments, wherein the liquid suspension comprises
(i) a pharmaceutical agent, wherein the pharmaceutical agent is a celastrol compound, a celastrol polymorph, or a celastrol analogue;
(ii) an emulsifier or surfactant;
(iii) a mucoadhesive or viscosifier; and
(iv) a preservative.

Embodiment 63. The method or pharmaceutical composition of any of the preceding embodiments, wherein the liquid suspension comprises:
(i) a pharmaceutical agent, wherein the pharmaceutical agent is a celastrol compound, a celastrol polymorph, or a celastrol analogue;
(ii) a polysorbate;
(iii) a cellulose; and
(iv) a preservative.

Embodiment 64. The method or pharmaceutical composition of any of the preceding embodiments, wherein the liquid suspension comprises a mucoadhesive or viscosifier (e.g., Hydroxypropyl methylcellulose (HPMC) or a combination of microcrystalline cellulose and sodium carboxymethyl cellulose (MCC-NaCMC).

Embodiment 65. The method or pharmaceutical composition of any of the preceding embodiments, wherein the liquid suspension comprises an emulsifier or surfactant (e.g., polysorbate 20) in an amount of from about 5 mg/mL to about 50 mg/mL (e.g., from about 5 mg/mL to about 40 mg/mL, from about 5 mg/mL to about 30 mg/mL, from about 10 mg/mL to about 30 mg/mL, or from about 20 mg/mL to about 30 mg/mL).

Embodiment 66. The method or pharmaceutical composition of any of the preceding embodiments, wherein the liquid suspension comprises an emulsifier or surfactant (e.g., polysorbate 20) in an amount of about 25 mg/mL.

Embodiment 67. The pharmaceutical composition of any of the preceding embodiments, wherein the liquid suspension comprises comprises the emulsifier or surfactant at a molar concentration of from about 5 mM to about 40 mM.

Embodiment 68. The pharmaceutical composition of any of the preceding embodiments, wherein the liquid suspension comprises comprises the emulsifier or surfactant at a molar concentration of from about 10 mM to about 30 mM.

Embodiment 69. The pharmaceutical composition of any of the preceding embodiments, wherein the liquid suspension comprises comprises the emulsifier or surfactant at a molar concentration of about 20 mM.

Embodiment 70. The method or pharmaceutical composition of any of the preceding embodiments, wherein the liquid suspension comprises a preservative (e.g., benzalkonium chloride) in an amount of up to about 1 mg/mL (e.g., up to about 0.1 mg/mL, up to about 0.2 mg/mL, up to about 0.3 mg/mL, up to about 0.4 mg/mL, up to about 0.5 mg/mL, up to about 0.6 mg/mL, up to about 0.7 mg/mL, up to about 0.8 mg/mL, or up to about 0.9 mg/mL).

Embodiment 71. The method or pharmaceutical composition of any of the preceding embodiments, wherein the liquid suspension comprises a preservative (e.g., benzalkonium chloride) in an amount of from about 0.01 mg/mL to 1.00 mg/mL (e.g., from about 0.01 mg/mL to about 0.8 mg/mL, from about 0.01 mg/mL to about 0.6 mg/mL, from about 0.01 mg/mL to about 0.4 mg/mL, from about 0.01 mg/mL to about 0.2 mg/mL, from about 0.05 mg/mL to about 0.2 mg/mL, or from about 0.05 mg/mL to about 0.15 mg/mL).

Embodiment 72. The method or pharmaceutical composition of any of the preceding embodiments, wherein the liquid suspension comprises a preservative (e.g., benzalkonium chloride) in an amount of about 0.1 mg/mL.

Embodiment 73. The pharmaceutical composition of any of the preceding embodiments, wherein the liquid suspension comprises the preservative at a molar concentration of from about 0.1 mM to about 0.5 mM.

Embodiment 74. The pharmaceutical composition of any of the preceding embodiments, wherein the liquid suspension comprises the preservative at a molar concentration of from about 0.2 mM to about 0.4 mM.

Embodiment 75. The pharmaceutical composition of any of the preceding embodiments, wherein the liquid suspension comprises the preservative at a molar concentration of about 0.3 mM.

Embodiment 76. The method or pharmaceutical composition of any of the preceding embodiments, wherein the liquid suspension comprises a mucoadhesive or viscosifier (e.g., HPMC or MCC-NaCMC) in an amount of from about 0.1 mg/mL to about 5 mg/mL (e.g, from about 0.1 mg/mL to about 4 mg/mL, from about 0.1 mg/mL to about 3 mg/mL, from about 0.1 mg/mL to about 2 mg/mL, or from about 0.5 mg/mL to about 1.5 mg/mL.

Embodiment 77. The method or pharmaceutical composition of any of the preceding embodiments, wherein the liquid suspension comprises a mucoadhesive or viscosifier (e.g., HPMC or MCC-NaCMC) in an amount of about 1 mg/mL.

Embodiment 78. The method or pharmaceutical composition of any of the preceding embodiments, wherein the liquid suspension comprises a mucoadhesive or viscosifier (e.g., HPMC or MCC-NaCMC) in an amount of from about 5 mg/mL to about 50 mg/mL (e.g, from about 5 mg/mL to about 40 mg/mL, from about 5 mg/mL to about 30 mg/mL, from about 10 mg/mL to about 30 mg/mL, from about 5 mg/mL to about 20 mg/mL, from about 10 mg/mL to about 20 mg/mL, or from about 15 mg/mL to about 20 mg/mL.

Embodiment 79. The method or pharmaceutical composition of any of the preceding embodiments, wherein the liquid suspension comprises a mucoadhesive or viscosifier (e.g., HPMC or MCC-NaCMC) in an amount of about 18 mg/mL.

Embodiment 80. The method or pharmaceutical composition of any of the preceding embodiments, wherein the liquid suspension comprises the pharmaceutical agent in an amount of from about 5 mg/mL to about 50 mg/mL (e.g, from about 5 mg/mL to about 40 mg/mL, from about 5 mg/mL to about 30 mg/mL, from about 10 mg/mL to about 30 mg/mL, or from about 15 mg/mL to about 25 mg/mL.

Embodiment 81. The method or pharmaceutical composition of any of the preceding embodiments, wherein the liquid suspension comprises the pharmaceutical agent in an amount of about 20 mg/mL.

Embodiment 82. The pharmaceutical composition of any of the preceding embodiments, wherein the liquid suspension comprises the pharmaceutical agent at a molar concentration of from about 10 mM to about 70 mM.

Embodiment 83. The pharmaceutical composition of any of the preceding embodiments, wherein the liquid suspension comprises the pharmaceutical agent at a molar concentration of from about 20 mM to about 60 mM.

Embodiment 84. The pharmaceutical composition of any of the preceding embodiments, wherein the liquid suspension comprises the pharmaceutical agent at a molar concentration of from about 40 mM to about 50 mM.

Embodiment 85. The pharmaceutical composition of any of the preceding embodiments, wherein the liquid suspension comprises the pharmaceutical agent at a molar concentration of about 44 mM.

Embodiment 86. The method or pharmaceutical composition of any of the preceding embodiments, wherein the liquid suspension comprises the pharmaceutical agent and an emulsifier or surfactant (e.g., polysorbate 20) in a wt/wt ratio of from about 1:0.6 to about 1:2.

Embodiment 87. The method or pharmaceutical composition of any of the preceding embodiments, wherein the liquid suspension comprises the pharmaceutical agent and an emulsifier or surfactant (e.g., polysorbate 20) in a wt/wt ratio of about 1:0.6, about 1:0.7, about 1:0.8, about 1:0.9, about 1:1.0, about 1:1.1, about 1:1.2, about 1:1.3, about 1:1.4, about 1:1.5, about 1:1.6, about 1:1.7, about 1:1.8, about 1:1.9, or about 1:2.0.

Embodiment 88. The method or pharmaceutical composition of any of the preceding embodiments, wherein the liquid suspension comprises the pharmaceutical agent and an emulsifier or surfactant (e.g., polysorbate 20) in a wt/wt ratio of about 1:1.3.

Embodiment 89. The method or pharmaceutical composition of any of the preceding embodiments, wherein the liquid suspension comprises the pharmaceutical agent and a preservative (e.g., benzalkonium chloride) in a wt/wt ratio of from about 1:0.001 to about 1:0.01 (e.g., about 1:1:0.001, about 1:0.002, about 1:0.003, about 1:0.004, about 1:0.005, about 1:0.006, about 1:0.007, about 1:0.008, about 1:0.009, or about 1:0.01).

Embodiment 90. The method or pharmaceutical composition of any of the preceding embodiments, wherein the liquid suspension comprises the pharmaceutical agent and a preservative (e.g., benzalkonium chloride) in a wt/wt ratio of about 1:0.005.

Embodiment 91. The method or pharmaceutical composition of any of the preceding embodiments, wherein the liquid suspension comprises the pharmaceutical agent, an emulsifier or surfactant (e.g., polysorbate 20) and a preservative (e.g., benzalkonium chloride) in a wt/wt ratio of about 1:1.3:0.005.

Embodiment 92. The method or pharmaceutical composition of any of the preceding embodiments, wherein the liquid suspension comprises the pharmaceutical agent and a mucoadhesive or viscosifier (e.g., HPMC or MCC-NaCMC) in a wt/wt ratio of from about 1:0.01 to about 1:0.1 (e.g., about 1:0.01, about 1:0.02, about 1:0.03, about 1:0.04, about 1:0.05, about 1:0.06, about 1:0.07, about 1:0.08, about 1:0.09, about 1:0.1).

Embodiment 93. The method or pharmaceutical composition of any of the preceding embodiments, wherein the liquid suspension comprises the pharmaceutical agent and a mucoadhesive or viscosifier (e.g., HPMC or MCC-NaCMC) in a wt/wt ratio of about 1:0.05.

Embodiment 94. The method or pharmaceutical composition of any of the preceding embodiments, wherein the liquid suspension comprises the pharmaceutical agent and a mucoadhesive or viscosifier (e.g., HPMC or MCC-NaCMC) in a wt/wt ratio of from about 1:0.3 to about 1:1.5 (e.g., about 1:0.3, about 1:0.4, about 1:0.5, about 1:0.6, about 1:7, about 1:0.8, about 1:0.9, about 1:1, about 1:1.1, about 1:1.2, about 1:1.3, about 1:1.4, or about 1:1.5).

Embodiment 95. The method or pharmaceutical composition of any of the preceding embodiments, wherein the liquid suspension comprises the pharmaceutical agent and a mucoadhesive or viscosifier (e.g., HPMC or MCC-NaCMC) in a wt/wt ratio of about 1:0.9.

Embodiment 96. The method or pharmaceutical composition of any of the preceding embodiments, wherein the liquid suspension comprises the pharmaceutical agent, an emulsifier or surfactant (e.g., polysorbate 20) and a mucoadhesive or viscosifier (e.g., HPMC or MCC-NaCMC) in a wt/wt ratio of about 1:1.3:0.05.

Embodiment 97. The method or pharmaceutical composition of any of the preceding embodiments, wherein the liquid suspension comprises the pharmaceutical agent, an emulsifier or surfactant (e.g., polysorbate 20) and a mucoadhesive or viscosifier (e.g., HPMC or MCC-NaCMC) in a wt/wt ratio of about 1:1.3:0.9.

Embodiment 98. The method or pharmaceutical composition of any of the preceding embodiments, wherein the emulsifier or surfactant in the liquid suspension is polysorbate 20.

Embodiment 99. The method or pharmaceutical composition of any of the preceding embodiments, wherein the preservative is benzalkonium chloride.

Embodiment 100. The method or pharmaceutical composition of any of the preceding embodiments, wherein the mucoadhesive or viscosifier is HPMC or MCC-NaCMC.

Embodiment 101. The method or pharmaceutical composition of any of the preceding embodiments, wherein the liquid suspension comprises the pharmaceutical agent, polysorbate 20, benzalkonium chloride and HPMC or MCC-NaCMC.

Embodiment 102. The method or pharmaceutical composition of any of the preceding embodiments, wherein the liquid suspension comprises the pharmaceutical agent, polysorbate 20, benzalkonium chloride and HPMC.

Embodiment 103. The method or pharmaceutical composition of any of the preceding embodiments, wherein the liquid suspension comprises the pharmaceutical agent, polysorbate 20, benzalkonium chloride and MCC-NaCMC.

Embodiment 104. The method or pharmaceutical composition of any of the preceding embodiments, wherein the pharmaceutical agent is a celastrol polymorph.

Embodiment 105. The method or pharmaceutical composition of any of the preceding embodiments, wherein the pharmaceutical composition is formulated for intranasal administration.

Embodiment 106. The method or pharmaceutical composition of any of the preceding embodiments, wherein the pharmaceutical composition is in the form of a nasal spray or nose drops.

Embodiment 107. The kit of any one of the preceding embodiments, wherein the kit comprises an intranasal dosage form comprising the celastrol compound, the celastrol polymorph, or the celastrol analogue, or a pharmaceutical composition comprising the celastrol compound, the celastrol polymorph, or the celastrol analogue.

Embodiment 108. The kit of any one of the preceding embodiments, wherein the kit comprises the celastrol compound, the celastrol polymorph, or the celastrol analogue, or a pharmaceutical composition comprising the celastrol compound, the celastrol polymorph, or the celastrol analogue in a solution, suspension, emulsion, powder, or gel composition for intranasal delivery.

Embodiment 109. The kit of any one of the preceding embodiments, wherein the kit comprises a nasal spray, a dropper bottle, or a metered dose spray pump.

Embodiment 110. The kit of any one of the preceding embodiments, wherein the nasal spray is an aqueous nasal spray, a hydroalcoholic nasal spray, or a nonaqueous-based nasal spray.

Embodiment 111. The kit of any one of the preceding embodiments, wherein the kit comprises a dry-powder nasal spray, dry-powder inhaler, or aspirator.

Embodiment 112. The kit of any one of the preceding embodiments, wherein the intranasal dosage form is a liquid suspension as described in any of the aspects and embodiments described herein.

Embodiment 113. The method of any one of the preceding embodiments, wherein the celastrol compound, the celastrol polymorph, or the celastrol analogue, or a pharmaceutical composition comprising the celastrol compound, the celastrol polymorph, or the celastrol analogue is administered to the subject daily, every two days, every three days, every four days, every five days, or every six days.

Embodiment 114. The method of any one of the preceding embodiments, wherein the celastrol compound, the celastrol polymorph, or the celastrol analogue, or a pharmaceutical composition comprising the celastrol compound, the celastrol polymorph, or the celastrol analogue is administered to the subject weekly, every two weeks, every three weeks, or monthly.

Embodiment 115. The method of any one of the preceding embodiments, wherein the celastrol compound, the celastrol polymorph, or the celastrol analogue, or a pharmaceutical composition comprising the celastrol compound, the celastrol polymorph, or the celastrol analogueis administered to the subject once per day, twice per day, three times per day, or four times per day.

Embodiment 116. The method of any one of the preceding embodiments, wherein the celastrol compound, the celastrol polymorph, or the celastrol analogue, or a pharmaceutical composition comprising the celastrol compound, the celastrol polymorph, or the celastrol analogue is administered to the subject once per week, twice per week, three times per week, four times per week, five times per week, or six times per week.

Embodiment 117. The method of any one of the preceding embodiments, wherein the celastrol compound, the celastrol polymorph, or the celastrol analogue, or a pharmaceutical composition comprising the celastrol compound, the celastrol polymorph, or the celastrol analogue is administered to the subject once per week or twice per week.

Embodiment 118. The method of any one of the preceding embodiments, wherein the celastrol compound, the celastrol polymorph, or the celastrol analogue, or a pharmaceutical composition comprising the celastrol compound, the celastrol polymorph, or the celastrol analogue is administered to the subject weekly.

Embodiment 119. The method of any one of the preceding embodiments, wherein the pharmaceutical composition, by itself or as included in the kit, is administered to the subject daily, every two days, every three days, every four days, every five days, or every six days.

Embodiment 120. The method of any one of the preceding embodiments, wherein the pharmaceutical composition, by itself or as included in the kit, is administered to the subject weekly, every two weeks, every three weeks, or monthly.

Embodiment 121. The method of any one of the preceding embodiments, wherein the pharmaceutical composition, by itself or as included in the kit, is administered to the subject once per day, twice per day, three times per day, or four times per day.

Embodiment 122. The method of any one of the preceding embodiments, wherein the pharmaceutical composition, by itself or as included in the kit, is administered to the subject once per week, twice per week, three times per week, four times per week, five times per week, or six times per week.

Embodiment 123. The method of any one of the preceding embodiments, wherein the pharmaceutical composition, by itself or as included in the kit, is administered to the subject once per week or twice per week.

Embodiment 124. The method of any one of the preceding embodiments, wherein the pharmaceutical composition, by itself or as included in the kit, is administered to the subject weekly.

Embodiment 125. The method of any one of the preceding embodiments, wherein the pharmaceutical composition, by itself or as included in the kit, is formulated for administration to the subject daily, every two days, every three days, every four days, every five days, or every six days.

Embodiment 126. The method of any one of the preceding embodiments, wherein the pharmaceutical composition, by itself or as included in the kit, is formulated for administration to the subject weekly, every two weeks, every three weeks, or monthly.

Embodiment 127. The method of any one of the preceding embodiments, wherein the pharmaceutical composition, by itself or as included in the kit, is formulated for administration to the subject once per day, twice per day, three times per day, or four times per day.

Embodiment 128. The method of any one of the preceding embodiments, wherein the pharmaceutical composition, by itself or as included in the kit, is formulated for administration to the subject once per week, twice per week, three times per week, four times per week, five times per week, or six times per week.

Embodiment 129. The method of any one of the preceding embodiments, wherein the pharmaceutical composition, by itself or as included in the kit, is formulated for administration to the subject once per week or twice per week.

Embodiment 130. The method of any one of the preceding embodiments, wherein the pharmaceutical composition, by itself or as included in the kit, is formulated for administration to the subject weekly.

Embodiment 131. The method of any one of the preceding embodiments, further comprising identifying or diagnosing a subject in need of treatment.

Embodiment 132. The method of any one of the preceding embodiments, wherein the identifying comprises administering a Hyperphagia Questionnaire (HQ).

Embodiment 133. The method of any one of the preceding embodiments, wherein the method further comprises conducting a clinical evaluation of the subject.

Embodiment 134. The method of any one of the preceding embodiments, wherein a clinical evaluation of the subject comprises a Dyken's Hyperphagia Questionnaire (HQ) score of 11 or greater and wherein the score is reduced to 0, 1, or 2.

Embodiment 135. The method of any one of the preceding embodiments, wherein a clinical evaluation of the subject comprises a Dyken's Hyperphagia Questionnaire (HQ) score of 11 or greater and wherein the score is reduced to 0, 1, or 2 up to 6 months following treatment.

Embodiment 136. The method of any one of the preceding embodiments, wherein a clinical evaluation of the subject comprises a Dyken's Hyperphagia Questionnaire (HQ) score of 11 or greater and wherein the score is reduced to 0, 1, or 2 up to 5 months following treatment.

Embodiment 137. The method of any one of the preceding embodiments, wherein a clinical evaluation of the subject comprises a Dyken's Hyperphagia Questionnaire (HQ) score of 11 or greater and wherein the score is reduced to 0, 1, or 2 up to 4 months following treatment.

Embodiment 138. The method of any one of the preceding embodiments, wherein a clinical evaluation of the subject comprises a Dyken's Hyperphagia Questionnaire (HQ) score of 11 or greater and wherein the score is reduced to 0, 1, or 2 up to 3 months following treatment.

Embodiment 139. The method of any one of the preceding embodiments, wherein a clinical evaluation of the subject comprises a Dyken's Hyperphagia Questionnaire (HQ) score of 11 or greater and wherein the score is reduced to 0, 1, or 2 up to 2 months following treatment.

Embodiment 140. The method of any one of the preceding embodiments, wherein a clinical evaluation of the subject comprises a Dyken's Hyperphagia Questionnaire (HQ) score of 11 or greater and wherein the score is reduced to 0, 1, or 2 up to 1 month following treatment.

Embodiment 141. The method of any one of the preceding embodiments, wherein the identifying comprises administering Dyken's HQ or a modification or variation thereof.

Embodiment 142. The method of any one of the preceding embodiments, wherein the identifying comprises administering Dyken's HQ.

Embodiment 143. The method of any one of the preceding embodiments, wherein the PWS is diagnosed with a Hyperphagia Questionnaire (HQ).

Embodiment 144. The method, pharmaceutical composition, or kit of any one of the preceding embodiments, wherein the celastrol compound, celastrol polymorph, or celastrol analogue is a compound of Formula (I):

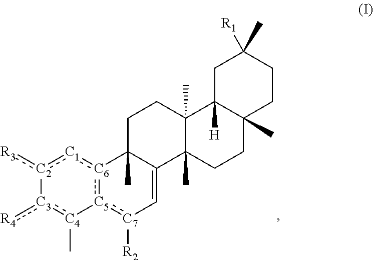

or a pharmaceutically acceptable salt, prodrug, or polymorph thereof, wherein
the dotted lines between C₁ and C₂, C₂ and R₃, C₃ and R₄, C₅ and C₆, C₅ and C₇, C₁ and C₆, and C₃ and C₄ indicate that a single or double bond may be present, as valence permits;
R₁ is —CN, —COOH, —COOCH₂CH₃, —CONHR₅, —CONR₅R₅, —COOR₅, —COOCH₃, —CH₂NR₅R₅, —CH₂OCONR₅R₅, —CH₂NR₅COOR₅, —CH₂R₅, —CH₂NR₅CONR₅R₅, —CH₂OH, —CH₂OR₅, alkylsulfate, alkylsulfonate, alkylphosphate, —CH₂OSO₃R₅, —CH₂OSO₂R₅, —CH₂OPO₃R₅R₅, —CH₂OPO₃HR₅, —CH₂OPO₃H₂, —C(═NR₅)NR₅R₅, —NR₅C(═NR₅)NR₅R₅, —CONH₂, —CH₂CONR₅R₅, —SR₅, —SO₃R₅, —SO₂R₅, —CH₂NHCOR₅, —CH₂NHCNR₅NR₅R₅, —CH₂COSR₅, CH₂NR₅COR₅, —CH₂NR₅CNR₅NR₅R₅, —CH₂NR₅COSR₅, —CH₂NHSO₂R₅, —CH₂NR₅SO₂R₅, —CHNR₅, —CHNOR₅, —H, —NH₂, —NHR₅, —NR₅R₅, —OH, —OR₅, phosphate, —OPO₃R₅R₅, —OPO₃HR₅, —OPO₃H₂, —NCO, —NCS, —N₃, —R₅, —C≡CR₅, —(CH═CH)R₅, —SH, —SR₅, —SO₂H, —SO₃H, —SO₂NR₅R₅, —SO₃R₅, —NHCOR₅, —, NHCNR₅NR₅R₅, —NHCOSR₅, secondary amide, tertiary amide, —NR₅COR₅, —NR₅C(═NH)NR₅R₅, —NR₅COSR₅, —NHC(═NR₅)R₅, —NR₅C(═NR₅)R₅, —NHSO₂ (NH₂), —NHSO₂R₅, —NR₅SO₂R₅, —NR₅SO₂NR₅R₅, —OCOR₅, —OCONRR₅, —O(C═O)OR₅, —SCOR₅, —O(C═NH)NR₅R₅, —OCSNHR₅, —OS(═O₂)R₅, —OS(═O₂)NR₅R₅, —SCONR₅R₅, —CH₂-aryl, —CH₂-heteroaryl,

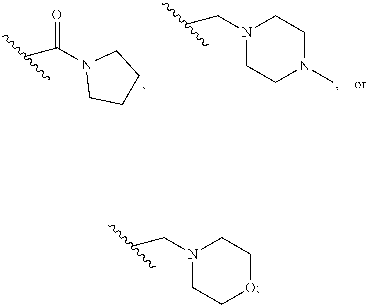

R₂ is —H, —CH₃, —SCH(CH₃)₂, —SC(═O)CH₃, —SC(═O)R₅, —SCH₂CH₂OCOCH₃, —SR₅, —SOR₅, —SOOR₅, —SCONR₅R₅,

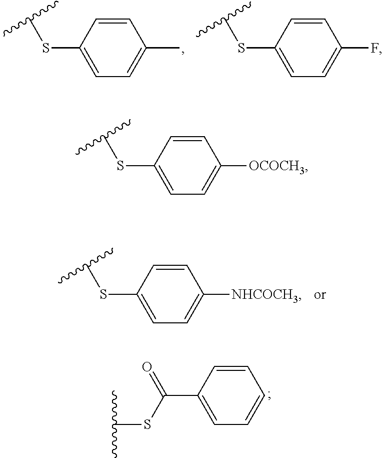

R₃ is —OCOCH₃, —OCOOCH₂CH₃, —OR₇, —R₇, or —NR₅R₅ when a double bond is present between C₁ and C₂, C₃ and C₄, and Cs and C₆
R₄ is —OCOCH₃, —OCOOCH₂CH₃, —OR₇, —R₇, or —NR₅R₅ when a double bond is present between C₁ and C₂, C₃ and C₄, and Cs and C₆;
R₃ is O when R₄ is O and a double bond is present between C₂ and R₃ and C₃ and R₄;
R₄ is —OCH₃, —OP(═O) (OCH₃)₂, —OH, —OCOOCH₂CH₃, —OCONHCH₂CH₃, —OCOOCH(CH₃)₂, —OR₇, —R₇, or —NR₅R₅ when R₃ is O and a double bond is present between C₂ and R₃; R₃ and R₄ may also be combined to form a heterocylic or carbocyclic ring;
R₅ is independently selected for each occurrence selected from hydrogen, an alkyl, cycloalkyl, alkoxy, heterocycloalkyl, alkylaryl, alkenyl, alkynyl, aryl, amine, and heteroaryl, wherein the heteroaryl is optionally substituted with substituents individually selected from alkyl, alkoxy, cycloalkyl, ether, halogen, hydroxyl, ether, cyano, nitrile, CF₃, ester, amide, cycloalkyl amide, sugar, urea, carbamate, thioether, sulfate, sulfonyl, sulfonic acid carboxylic acid, aryl, amine optionally substituted with one or more alkyl, and heteroarylamide optionally substituted with alkyl and/or alkoxy; or two R₅ groups taken together to form a cycloalkyl, heterocycloalkyl, aryl or heteraryl group, optionally substituted with substituents individually selected from alkyl, cycloalkyl, alkoxy, heterocycloalkyl, alkylaryl, alkenyl, alkynyl, aryl, heteroaryl, amine, halogen, hydroxyl, ether, nitrile, cyano, nitro, CF₃, ester amide, urea, carbamate, thioether, and a carboxylic acid group; and
R₇ is hydrogen, an alkyl, cycloalkyl, heterocycloalkyl, alkylaryl, alkenyl, alkynyl, aryl, or heteroaryl, optionally substituted with substituents individually selected from alkyl, cycloalkyl, ether, amine, halogen, hydroxyl, ether, nitrile, cyano, nitrile, CF₃, ester, amide, urea, carbamate, thioether, and carboxylic acid.

Embodiment 145. The method, pharmaceutical composition, or kit of any one of the preceding embodiments, wherein in the compound of Formula (I), R₁ is-NR₅C(═NR₅)NR₅R₅, —SR₅, —SO₃R₅, —SO₂R₅, —NH₂, —NHR₅, —NR₅R₅, —OH, —OR₅, —NCO, —NCS, —N₃, —SH, —SR₅, —SO₂H, —SO₃H, —SO₂NR₅R₅, —SO₃R₅, —NHCOR₅, —NHCNR₅NR₅R₅, —NHCOSR₅, —NR₅COR₅, —NR₅C(═NH)NR₅R₅, —NR₅COSR₅, —NHC(═NR₅)R₅, —NR₅C(═NR₅)R₅, —NHSO₂ (NH₂), —NHSO₂R₅, —NR₅SO₂R₅, —NR₅SO₂NR₅R₅, —OCOR₅, —OCONR₅R₅, —O(C═O)OR₅, —SCOR₅, —O(C═NH)NR₅R₅, —OCSNHR₅, —OS(═O₂)R₅, —OS(═O₂)NR₅R₅, or —SCONR₅R₅.

Embodiment 146. The method, pharmaceutical composition, or kit of any one of the preceding embodiments, wherein in the compound of Formula (I), is a compound of Formula (I)-a:

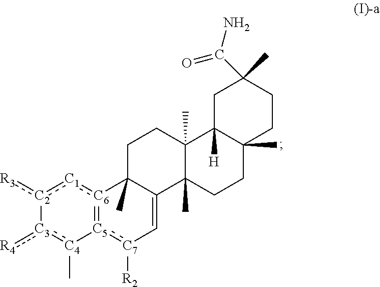

wherein each R₂, R₃, and R₄ is defined in Formula (I).

Embodiment 147. The method, pharmaceutical composition, or kit of any one of the preceding embodiments, wherein in the compound of Formula (I), or (I)-a, R₂ is —H, —SCH(CH₃)₂, —SC(═O)CH₃,

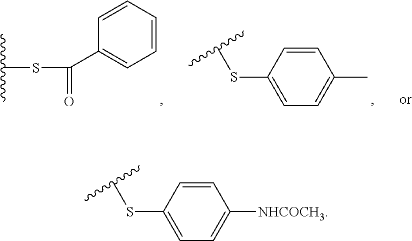

Embodiment 148. The method, pharmaceutical composition, or kit of any one of the preceding embodiments, wherein in the compound of Formula (I), or (I)-a, R₃ is —OC(═O)CH₃ or —OC(═O)OCH₂CH₃.

Embodiment 149. The method, pharmaceutical composition, or kit of any one of the preceding embodiments, wherein in the compound of Formula (I), or (I)-a, R₄ is —OC(═O)CH₃ or —OC(═O)OCH₂CH₃.

Embodiment 150. The method, pharmaceutical composition, or kit of any one of the preceding embodiments, wherein in the compound of Formula (I), R₁ is —COOH or —COOCH₃.

Embodiment 151. The method, pharmaceutical composition, or kit of any one of the preceding embodiments, wherein in the compound of Formula (I), or (I)-a, R₂ is —CH₃, —SC(═O)CH₃, —SCH(CH₃)₂, or —SCH₂CH₂OCOCH₃.

Embodiment 152. The method, pharmaceutical composition, or kit of any one of the preceding embodiments, wherein in the compound of Formula (I), or (I)-a, R₃ is —OCOCH₃ or —OH.

Embodiment 153. The method, pharmaceutical composition, or kit of any one of the preceding embodiments, wherein in the compound of Formula (I), or (I)-a, R₄ is —OCOCH₃ or —OH.

Embodiment 154. The method, pharmaceutical composition, or kit of any one of the preceding embodiments, wherein in the compound of Formula (I) is a compound of Formula (I)-b or Formula (I)-c:

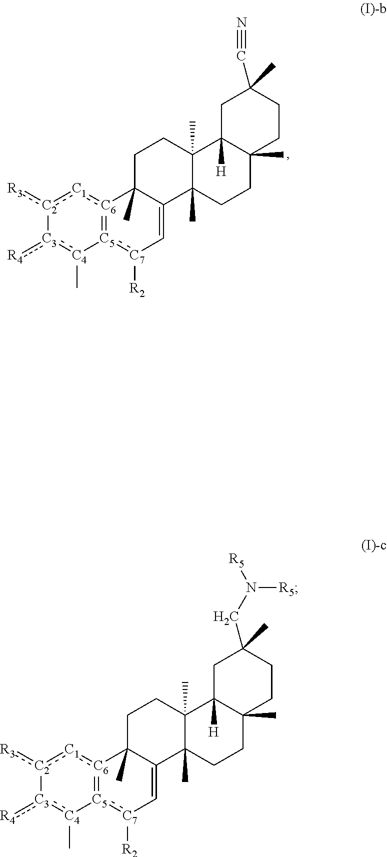

wherein each R₂, R₃, R₄ and R₅ is defined in Formula (I)

Embodiment 155. The method, pharmaceutical composition, or kit of any one of the preceding embodiments, wherein in the compound of Formula (I), (I)-a, (I)-b, or (Ic), R₂ is-SCH(CH₃)₂, —SC(═O)CH₃, —H,

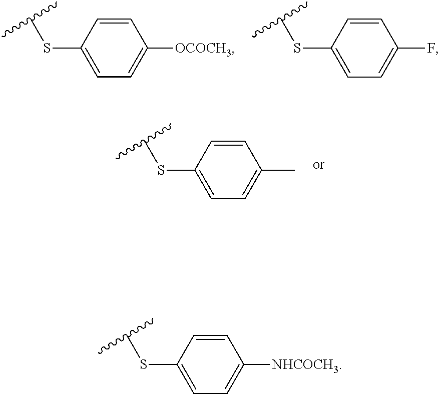

Embodiment 156. The method, pharmaceutical composition, or kit of any one of the preceding embodiments, wherein in the compound of Formula (I), (I)-a, (I)-b, or (Ic), R₃ is —OCOCH₃.

Embodiment 157. The method, pharmaceutical composition, or kit of any one of the preceding embodiments, wherein in the compound of Formula (I), (I)-a, (I)-b, or (Ic), R₄ is —OCOCH₃.

Embodiment 158. The method, pharmaceutical composition, or kit of any one of the preceding embodiments, wherein in the compound of Formula (I), (I)-a, (I)-b, or (Ic), R₃ and R₄ form a five membered-heterocycle comprising —OCOO—.

Embodiment 159. The method, pharmaceutical composition, or kit of any one of the preceding embodiments, wherein in the compound of Formula (I), (I)-a, (I)-b, or (Ic), R₅ is alkyl, preferably CH₃.

Embodiment 160. The method, pharmaceutical composition, or kit of any one of the preceding embodiments, wherein in the compound of Formula (I), (I)-a, (I)-b, or (Ic), R₃ is O and a double bond is present between C₂ and R₃ and C₃ and C₄.

Embodiment 161. The method, pharmaceutical composition, or kit of any one of the preceding embodiments, wherein in the compound of Formula (I), (I)-a, (I)-b, or (Ic), R₁ is-C(═O)OCH₂CH₃, —CN, —CONH₂, —CH₂N(CH₃)₂,

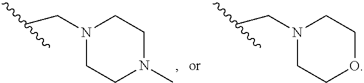

Embodiment 162. The method, pharmaceutical composition, or kit of any one of the preceding embodiments, wherein in the compound of Formula (I), (I)-a, (I)-b, or (Ic), R₂ is —H.

Embodiment 163. The method, pharmaceutical composition, or kit of any one of the preceding embodiments, wherein in the compound of Formula (I), (I)-a, (I)-b, or (Ic), R₄ is —OCH₃, —OP(═O) (OCH₃)₂, —OCH₂CH₃, —OH, —OCONHCH₂CH₃, —OCH₂COOCH₃, —OCOOCH₂CH₃, —OCH₂CH₂OH, —OCOOCH(CH₃)₂, or —OCH(CH₃)₂.

Embodiment 164. The method, pharmaceutical composition, or kit of any one of the preceding embodiments, wherein in the compound of Formula (I), (I)-a, (I)-b, or (Ic), R₃ and R₄ are O and a double bond is present between C₂ and R₃ and C₃ and R₄.

Embodiment 165. The method, pharmaceutical composition, or kit of any one of the preceding embodiments, wherein in the compound of Formula (I), (I)-a, (I)-b, or (Ic), R₁ is COOH, COOCH₃, or

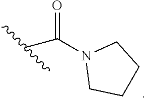 [SMILES: *C(=O)N1CCCC1]

Embodiment 166. The method, pharmaceutical composition, or kit of any one of the preceding embodiments, wherein in the compound of Formula (I), (I)-a, (I)-b, or (Ic), R₂ is CH₃.

Embodiment 167. The method, pharmaceutical composition, or kit of any one of the preceding embodiments, wherein in the compound of Formula (I), (I)-a, (I)-b, or (Ic), R₁ is —NR₅C(═NR₅)NR₅R₅, —SR₅, —SO₃R₅, —SO₂R₅, —NH₂, —NHR₅, —NR₅R₅, —OH, —OR₅, —NCO, —NCS, —N₃, —SH, —SR₅, —SO₂H, —SO₃H, —SO₂NR₅R₅, —SO₃R₅, —NHCOR₅, —NHCNR₅NR₅R₅, —NHCOSR₅, —NR₅COR₅, —NR₅C(═NH)NR₅R₅, —NR₅COSR₅, —NHC(═NR₅)R₅, —NR₅C(═NR₅)R₅, —NHSO₂ (NH₂), —NHSO₂R₅, —NR₅SO₂R₅, —NR₅SO₂NR₅R₅, —OCOR₅, —OCONR₅R₅, —O(C═O)OR₅, —SCOR₅, —O(C═NH)NR₅R₅, —OCSNHR₅, —OS(═O₂)R₅, —OS(═O₂)NR₅R₅, —SCONR₅R₅.

Embodiment 168. The method, pharmaceutical composition, or kit of any one of the preceding embodiments, wherein in the compound of Formula (I), (I)-a, (I)-b, or (Ic), R₂ is H.

Embodiment 169. The method, pharmaceutical composition, or kit of any one of the preceding embodiments, wherein in the compound of Formula (I), (I)-a, (I)-b, or (Ic), R₄ is OH, —OR₇, or —R₇ when R₃ is O and a double bond is present between C₂ and R₃.

Embodiment 170. The method, pharmaceutical composition, or kit of any one of the preceding embodiments, wherein the celastrol polymorph is crystalline Form III of celastrol, wherein the Form III of celastrol is characterized by an x-ray powder diffraction pattern comprising one or more peaks at 2θ angles (±0.2) selected from the group consisting of 9.51°, 14.81°, and 16.84°.

Embodiment 171. The method, pharmaceutical composition, or kit of any one of the preceding embodiments, wherein the celastrol polymorph is crystalline Form III of celastrol, wherein the Form III of celastrol is characterized by an X-ray powder diffraction pattern comprising one or more peaks at 2θ angles (±0.2) selected from the group consisting of 13.60°, 14.65°, 18.74°, and 19.10°.

Embodiment 172. The method, pharmaceutical composition, or kit of any one of the preceding embodiments, wherein the celastrol polymorph is crystalline Form III of celastrol, wherein the Form III of celastrol is characterized by an x-ray powder diffraction pattern comprising two peaks at 2θ angles (±0.2) selected from the group consisting of 9.51°, 14.81°, and 16.84°.

Embodiment 173. The method, pharmaceutical composition, or kit of any one of the preceding embodiments, wherein the celastrol polymorph is crystalline Form III of celastrol, wherein the Form III of celastrol is characterized by an X-ray powder diffraction pattern comprising two peaks at 2θ angles (±0.2) selected from the group consisting of 13.60°, 14.65°, 18.74°, and 19.10°.

Embodiment 174. The method, pharmaceutical composition, or kit of any one of the preceding embodiments, wherein the celastrol polymorph is crystalline Form III of celastrol, wherein the Form III of celastrol is characterized by an x-ray powder diffraction pattern comprising three peaks at 2θ angles (±0.2) selected from the group consisting of 9.51°, 14.81°, and 16.84°.

Embodiment 175. The method, pharmaceutical composition, or kit of any one of the preceding embodiments, wherein the celastrol polymorph is crystalline Form III of celastrol, wherein the Form III of celastrol is characterized by an X-ray powder diffraction pattern comprising three peaks at 2θ angles (±0.2) selected from the group consisting of 13.60°, 14.65°, 18.74°, and 19.10°.

Embodiment 176. The method, pharmaceutical composition, or kit of any one of the preceding embodiments, wherein the celastrol polymorph is crystalline Form III of celastrol, wherein the Form III of celastrol is characterized by a differential scanning calorimetry (DSC) profile with no endothermic peak and an exothermic peak at about 215° C. (onset).

Embodiment 177. The method, pharmaceutical composition, or kit of any one of the preceding embodiments, wherein the celastrol polymorph is crystalline Form III of celastrol, wherein the Form III of celastrol is characterized by a thermal gravimetric analysis (TGA) profile with about 0.5% weight loss at about the exothermic peak at about 215° C. (onset).

Embodiment 178. The method, pharmaceutical composition, or kit of any one of the preceding embodiments, wherein the celastrol polymorph is crystalline Form III of celastrol, wherein the Form III of celastrol is characterized by a differential scanning calorimetry (DSC) profile with no endothermic peak. In embodiments, the anhydrous Form III crystalline solid form is characterized by a differential scanning calorimetry (DSC) profile with an exothermic peak at about 215° C. (onset). In embodiments, the anhydrous Form III crystalline solid form is characterized by a differential scanning calormetry (DSC) profile with no endothermic peak but with an exothermic peak at about 215° C. (onset).

Embodiment 179. The method of any one of the preceding embodiments, wherein the method further comprises administering one or more additional therapeutic agents, wherein the one or more additional therapeutic agents comprises an anti-anxiety or anti-depression agent.

Embodiment 180. The pharmaceutical composition of any one of the preceding embodiments, wherein the pharmaceutical composition further comprises one or more additional therapeutic agents, wherein the one or more additional therapeutic agents comprises an anti-anxiety or anti-depression agent.

Embodiment 181. The method of any one of the preceding embodiments, wherein the anti-anxiety or anti-depression agent comprises a serotonin selective reuptake inhibitor (SSRI), a serotonin norepinephrine reuptake inhibitor (SNRI), a benzodiazepine (BZD), a tricyclic antidepressant (TCA), a monoamine oxidase (MAO) inhibitor (MAOI), or an antihistamine.

Embodiment 182. The method of any one of the preceding embodiments, wherein said anti-anxiety agent comprises a serotonin selective reuptake inhibitor (SSRI), a serotonin norepinephrine reuptake inhibitor (SNRI), a benzodiazepine (BZD), a tricyclic antidepressant (TCA), a monoamine oxidase (MAO) inhibitor (MAOI), or an antihistamine.

Embodiment 183. The method of any one of the preceding embodiments, wherein the anti-anxiety agent comprises escitalopram, fluoxetine, fluvoxamine, paroxetine, sertraline, duloxetine, venlafaxine, alprazolam, chlordiazepoxide, clonazepam, diazepam, lorazepam, oxazepam, clomipramine, doxepine, imipramine, phenelzine, hydroxyzine, or buspirone.

Embodiment 184. The method of any one of the preceding embodiments, wherein the anti-depression agent comprises fluoxetine hydrochloride, amitriptyline, or clonidine hydrochloride.

Embodiment 185. The method of any one of the preceding embodiments, wherein the method further comprises administering one or more additional therapeutic agents selected from:

| Activity | Compounds/Therapeutic Agent |
|---|---|
| Anxiety/antidepressant | Fluoxetine hydrochloride (e.g., |
| medications | PROZAC ®) |
| | Amitriptyline |
| | Clonidine hydrochloride |
| Sleep medications/Sleep related/ | Melatonin (e.g., modified-release) |
| Stimulants | Methylphenidate hydrochloride |
| | Methylphenidate hydrochloride, (e.g., |
| | extended-release) |
| | Dexamfetamine sulphate |
| | Modafinil |
| Anti-psychotic medications for | Risperidone |
| behavior or psychiatric disorders/ | Aripiprazole |
| mood stabilizers | Ziprasidone |
| | Olanzapine |
| | Quetiapine |
| | Haloperidol |
| | Lithium Carbonate |
| Anti-convulsant/anti-epileptic | Valproic Acid (e.g., Sodium |
| | Valproate) |
| medications for mood stabilizing | Lamotrigine |
| or behavior | Topirimate |
| Benzodiazepines (e.g., fast acting | Lorazepam (e.g., Ativan) |
| anxiety reducing sedatives) | |
| Growth Hormone | Somatropin |
| | Genotropin |
| | Omnitrope |
| | Norditropin ® FlexPro |

Embodiment 186. The method of any one of the preceding embodiments, wherein the method further comprises administering a conmed.

Embodiment 187. The method of any one of the preceding embodiments, wherein the method further comprises administering one or more additional therapeutic agents, wherein the one or more additional therapeutic agents comprises an anxiety or antidepressant medication, a sleep medication, a sleep related medications, a stimulant, an anti-psychotic medications for behavior or psychiatric disorders, a mood stabilizer, an anti-convulsant, an anti-epileptic medication for mood stabilizing or behavior, an benzodiazepine, a growth hormone, or combinations thereof.

Embodiment 188. The method of any one of the preceding embodiments, wherein the anxiety or antidepressant medication is selected from fluoxetine hydrochloride, amitriptyline, and clonidine hydrochloride, and wellbutrin.

Embodiment 189. The method of any one of the preceding embodiments, wherein the sleep medication, sleep related medication, or stimulant, is selected from modified-release melatonin, methylphenidate hydrochloride, methylphenidate hydrochloride extended-release, dexamfetamine sulphate, and modafinil.

Embodiment 190. The method of any one of the preceding embodiments, wherein the anti-psychotic medication for behavior or psychiatric disorders or mood stabilizer is selected from risperidone, aripiprazole, ziprasidone, olanzapine, quetiapine, haloperidol, and lithium carbonate.

Embodiment 191. The method of any one of the preceding embodiments, wherein the anti-convulsant or anti-epileptic medication for mood stabilizing or behavior is selected from valproic acid/sodium valproate, lamotrigine, and topirimate.

Embodiment 192. The method of any one of the preceding embodiments, wherein the benzodiazepine is lorazepam.

Embodiment 193. The method of any one of the preceding embodiments, wherein the growth hormone is selected from somatropin, genotropin, and omnitrope.

Embodiment 194. The method of any one of the preceding embodiments, wherein the celastrol compound, the celastrol polymorph, or the celastrol analogue and the one or more additional therapeutic agents are formulated in the same dosage unit.

Embodiment 195. The method of any one of the preceding embodiments, wherein the celastrol compound, the celastrol polymorph, or the celastrol analogue and the one or more additional therapeutic agents are formulated in separate dosage units.

Embodiment 196. The method of any one of the preceding embodiments, wherein the celastrol compound, the celastrol polymorph, or the celastrol analogue and the one or more additional therapeutic agents are administered simultaneously.

Embodiment 197. The method of any one of the preceding embodiments, wherein the celastrol compound, the celastrol polymorph, or the celastrol analogue and the one or more additional therapeutic agents are administered sequentially.

Embodiment 198. The method of any one of the preceding embodiments, wherein the celastrol compound, the celastrol polymorph, or the celastrol analogue or the composition comprising the celastrol compound, the celastrol polymorph, or the celastrol analogue is administered to the subject at a dose of from about 0.001 mg to about 10 mg per kg of body weight.

Embodiment 199. The method of any one of the preceding embodiments, wherein the celastrol compound, the celastrol polymorph, or the celastrol analogue or the composition comprising the celastrol compound, the celastrol polymorph, or the celastrol analogue is administered to the subject at a dose of from about 0.005 mg to about 10 mg per kg of body weight.

Embodiment 200. The method of any one of the preceding embodiments, wherein the celastrol compound, the celastrol polymorph, or the celastrol analogue or the composition comprising the celastrol compound, the celastrol polymorph, or the celastrol analogue is administered to the subject at a dose of from about 0.5 mg to 8 mg.

Embodiment 201. The method of any one of the preceding embodiments, wherein the celastrol compound, the celastrol polymorph, or the celastrol analogue or the composition comprising the celastrol compound, the celastrol polymorph, or the celastrol analogue is administered to the subject at a dose of from about 0.001 mg to about 0.5 mg per kg.

Embodiment 202. The method of any one of the preceding embodiments, wherein the celastrol compound, the celastrol polymorph, or the celastrol analogue or the composition comprising the celastrol compound, the celastrol polymorph, or the celastrol analogue is administered to the subject at a dose of from about 0.005 mg to about 0.5 mg per kg.

Embodiment 203. The method of any one of the preceding embodiments, wherein the celastrol compound, the celastrol polymorph, or the celastrol analogue or the composition comprising the celastrol compound, the celastrol polymorph, or the celastrol analogue is administered to the subject at a dose of from about 0.5 mg to about 4 mg or from about 0.5 to about 8 mg.

Embodiment 204. The method of any one of the preceding embodiments, wherein the celastrol compound, the celastrol polymorph, or the celastrol analogue or the composition comprising the celastrol compound, the celastrol polymorph, or the celastrol analogue is administered to the subject once a week or twice a week.

Embodiment 205. The method of any one of the preceding embodiments, wherein the celastrol compound, the celastrol polymorph, or the celastrol analogue is administered to the subject 3, 4, 6, or 7 times a week.

Embodiment 206. The method of any one of the preceding embodiments, wherein the celastrol compound, the celastrol polymorph, or the celastrol analogue or the composition comprising the celastrol compound, the celastrol polymorph, or the celastrol analogue is administered to the subject once every 8, 9, 10, 11, 12, 13, 14, 15, 20, 21, 28 30 or 31 days.

Embodiment 207. The method of any one of the preceding embodiments, wherein said compulsive behavior is reduced by at least 10% following treatment.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims. All publications and patent applications cited in this specification are herein incorporated by reference as if each individual publication or patent application were specifically and individually indicated to be incorporated by reference.

What is claimed is:

1. A method of treating a disorder characterized by or associated with a compulsive behavior in a subject comprising administering a pharmaceutical composition comprising a celastrol compound, a celastrol polymorph, or a celastrol analogue to the subject.

2. A method for treating Prader-Willi Syndrome (PWS), comprising identifying a subject comprising PWS and administering a pharmaceutical composition comprising a celastrol compound, a celastrol polymorph, or a celastrol analogue to said subject, wherein a PWS-associated compulsive behavior is reduced following treatment.

3. A method of treating a Prader Willi Syndrome (PWS)-associated compulsive behavior in a subject comprising administering a pharmaceutical composition comprising a celastrol compound, a celastrol polymorph, or a celastrol analogue to the subject.

4. A method of treating Prader Willi Syndrome (PWS) in a subject comprising administering a pharmaceutical composition comprising a celastrol compound, a celastrol polymorph, or a celastrol analogue to the subject, wherein the treating PWS comprises reducing a compulsive behavior associated with PWS.

5. The method of any one of claims 1-4 wherein the compulsive behavior is hyperphagia.

6. The method of claim 2, wherein the identifying comprises administering a Hyperphagia Questionnaire (HQ) to the subject.

7. The method of any one of the preceding claims, wherein said pharmaceutical composition is a liquid suspension.

8. The method of claim 7, wherein the liquid suspension comprises a emulsifier or surfactant; a preservative; and a mucoadhesive or viscosifier.

9. The method of claim 8, wherein the emulsifier or surfactant is present in an amount of from about 5 mg/mL to about 50 mg/mL; the preservative is present in an amount of up to about 1 mg/mL; and a mucoadhesive or viscosifier is present in an amount of from about 0.1 mg/mL to about 5 mg/mL.

10. The method of claim 8, wherein the emulsifier or surfactant is present in an amount of from about 5 mg/mL to about 50 mg/mL; the preservative is present in an amount of up to about 1 mg/mL; and a mucoadhesive or viscosifier is present in an amount of from about 5 mg/mL to about 50 mg/mL.

11. The method of claim 8, wherein the liquid suspension comprises the emulsifier or surfactant at a molar concentration of from about 5 mM to about 40 mM; the preservative the preservative at a molar concentration of from about 0.1 mM to about 0.5 mM; and the pharmaceutical agent at a molar concentration of from about 20 mM to about 60 mM.

12. The method of claim 7, wherein the liquid suspension comprises polysorbate 20, benzalkonium chloride, and HPMC or MCC-NaCMC.

13. The method of any one of claims 1-12, wherein the pharmaceutical composition is administered intranasally.

14. The method of any one of the preceding claims, further comprising administering one or more additional therapeutic agents, wherein the one or more additional therapeutic agents comprises an anti-anxiety or anti-depression agent.

15. The method of claim 14, wherein said anti-anxiety agent comprises a serotonin selective reuptake inhibitor (SSRI), a serotonin norepinephrine reuptake inhibitor (SNRI), a benzodiazepine (BZD), a tricyclic antidepressant (TCA), a monoamine oxidase (MAO) inhibitor (MAOI), or an antihistamine.

16. The method of claim 14, wherein the anti-anxiety agent is escitalopram, fluoxetine, fluvoxamine, paroxetine, sertraline, duloxetine, venlafaxine, alprazolam, chlordiazepoxide, clonazepam, diazepam, lorazepam, oxazepam, clomipramine, doxepine, imipramine, phenelzine, hydroxyzine, or buspirone.

17. The method of claim 14, wherein the anti-depression agent comprises Fluoxetine hydrochloride, Amitriptyline, or Clonidine hydrochloride.

18. The method of any one of claims 1-6 and 14-17, wherein the pharmaceutical composition is in the form of a capsule or tablet and is administered to the subject by oral administration.

19. The method of any one of claims 1-6 and 14-17, wherein the pharmaceutical composition is administered by oral, subcutaneous, or intravenous administration.

20. The method of any one of the preceding claims, wherein the pharmaceutical composition is administered to the subject in an amount corresponding to a dose of the celastrol compound, celastrol polymorph, or celastrol analogue of from about 0.001 mg to about 10 mg per kg of body weight.

21. The method of claim 20, wherein the pharmaceutical composition is administered to the subject in an amount of the celastrol compound, celastrol polymorph, or celastrol analogue corresponding to an amount of the celastrol compound of from about 0.5 mg to about 8 mg.

22. The method of any one of the preceding claims, wherein the pharmaceutical composition is administered to the subject once a week or twice a week.

23. The method of any one of the preceding claims, wherein the subject is not obese.

24. The method of any one of the preceding claims, wherein the subject is pre-obese, obese, or morbidly obese.

25. The method of any one of the preceding claims, wherein said compulsive behavior is reduced by at least 10% following treatment.

26. The method of any one of claims 2 and 5-25, wherein the identifying a subject comprises conducting a clinical evaluation of the subject and wherein the clinical evaluation of the subject comprises a Dyken's Hyperphagia Questionnaire (HQ) score of 11 or greater and wherein the score is reduced to 0, 1, or 2 following treatment.

27. The method of claim 26, wherein the score is reduced for up to 6 months following treatment.

28. A pharmaceutical composition for preventing or treating PWS in a subject, comprising a therapeutically effective amount of a celastrol compound.

29. A kit for preventing or treating PWS in a subject, comprising the pharmaceutical composition of claim 28, an oral or intranasal applicator, and instructions for use.
